(19) United States
(12) Patent Application Publication
(10) Pub. No.: US 2025/0177569 A1
(43) Pub. Date: Jun. 5, 2025

(54) COMPOSITIONS FOR THE MODIFICATION OF THE HUMAN NRAS AND KRAS GENES

(71) Applicant: Mammoth Biosciences, Inc., Brisbane, CA (US)

(72) Inventors: Subhadra JAYARAMAN RUKMINI, Redwood City, CA (US); Shreyans SADANGI, Burlingame, CA (US); Lucas Benjamin HARRINGTON, San Francisco, CA (US); Pei-Qi LIU, Oakland, CA (US); Benjamin Julius RAUCH, San Francisco, CA (US); William Douglass WRIGHT, Fairfield, CA (US); Stepan TYMOSHENKO, Sacramento, CA (US); Wiputra Jaya HARTONO, San Francisco, CA (US); Aaron DELOUGHERY, San Francisco, CA (US)

(21) Appl. No.: 18/966,406

(22) Filed: Dec. 3, 2024

Related U.S. Application Data
(60) Provisional application No. 63/605,619, filed on Dec. 4, 2023.

(51) Int. Cl. A61K 48/00; A61P 35/00; C12N 9/22; C12N 15/11; C12N 15/86

(52) U.S. Cl. CPC A61K 48/005; A61P 35/00; C12N 9/22; C12N 15/111; C12N 15/86; C12N 2310/20; C12N 2310/315; C12N 2310/321; C12N 2750/14143

(57) ABSTRACT

Provided herein are compositions, systems and methods for allele-specific editing of a target gene in a cell, comprising contacting the cell with an effector protein or a polynucleotide encoding the same, and a guide nucleic acid or a polynucleotide encoding the same. These effector proteins may be characterized as CRISPR-associated (Cas) proteins. Various compositions, systems, and methods of the present disclosure may leverage the activities of these effector proteins for the modification, detection, and engineering the NRAS or KRAS gene.

### CROSS-REFERENCE TO RELATED APPLICATIONS

The present application claims priority to U.S. Provisional Application 63/605,619, filed Dec. 4, 2023, which is herein incorporated by reference in its entirety.

### SEQUENCE LISTING

The contents of the electronic sequence listing (MABI_047_01US_SeqList_ST26.xml; Size: 183,342 bytes; and Date of Creation: Nov. 7, 2024) are herein incorporated by reference in its entirety.

### BACKGROUND

Allele-specific gene disruption induced by non-homologous end-joining (NHEJ) DNA repair offers a potential treatment option for autosomal dominant diseases. The mutant allele can be disrupted by CRISPR-Cas using a guide-specific approach, where the missense mutation is located within the guide sequence. Alternatively, if the mutation forms a novel protospacer adjacent motif (PAM), then CRISPR-Cas nucleases can disrupt the mutant allele by a PAM-specific approach. However, it remains challenging to discriminate many single-nucleotide mutations from wild-type alleles because of the low specificity of the current CRISPR Cas nucleases being used. Therefore, it is crucial to develop a highly specific Cas nuclease system that can discriminate single-nucleotide mutations.

### SUMMARY

Neuroblastoma RAS viral oncogene homolog (NRAS) is part of the MAPK signaling pathway in mammalian cells, responsible for energy conversion and metabolism. NRAS Q61L is an oncogenic mutation developed in the NRAS protein, which renders the protein constitutively active, and subsequently leads to unchecked proliferation of the cell. Targeting the mutant NRAS has the potential to reduce cell proliferation and induces apoptosis in cancer cells. In some aspects, the present disclosure provides compositions, systems and methods for treating disorders associated with NRAS.

Kirsten rat sarcoma virus (KRAS) is a GTPase that is involved in checkpoints for cell proliferation. Mutations in the KRAS gene can result in a protein that is constitutively in its GTP-bound active state. The constant activation of KRAS promotes the continuous transmission of proliferative signals within the cell, leading to uncontrolled cell division, a hallmark of cancer. KRAS mutations are frequently observed in various cancers. The KRAS gene is mutated in more than 90% of pancreatic cancers and more than 30% of colon and lung cancers. Targeting the mutant KRAS has the potential to reduce cell proliferation and induces apoptosis in cancer cells. In some aspects, the present disclosure provides compositions, systems and methods for treating disorders associated with KRAS.

In some aspects, disclosed herein are methods for allele-specific editing of a target gene in a cell, comprising contacting the cell with an effector protein or a polynucleotide encoding the same, and a guide nucleic acid or a polynucleotide encoding the same, wherein the effector protein comprises a CasPhi.12 or a variant thereof, or a CasM.265466 or a variant thereof, and wherein the guide nucleic acid comprises a sequence that is complementary to a target sequence in a mutant allele of the target gene. In some embodiments, the effector protein comprises a sequence that is at least 80%, at least 85%, at least 90%, at least 95%, at least 97%, at least 98%, at least 99%, or 100% identical to any one of SEQ ID NOs: 1-4, 24-43, and 44-61. In some embodiments, the guide nucleic acid comprises a spacer sequence comprising a nucleotide sequence that is complementary to the target sequence comprising a missense mutation on the mutant allele. In some embodiments, the guide nucleic acid comprises a spacer sequence comprising a nucleotide sequence that is complementary to the target sequence that is adjacent to a mutated protospacer adjacent motif (PAM) specific for the mutant allele. In some embodiments, the guide nucleic acid comprises a modification of at least one nucleotide or internucleotide linkage. In some embodiments, the modification is selected from a 2′ O-methyl, a 2′-fluoro, a locked nucleic acid (LNA), a peptide nucleic acid (PNA), a phosphorothioate linkage, and a 5′ cap, and a combination thereof. In some embodiments, the modification comprises 2′ O-Methyl modifications of the first 3 nucleotides and last 3 nucleotides of the guide nucleic acidsequence, phosphorothioate linkages between the first 4 nucleotides of the guide nucleic acidsequence, and phosphorothioate linkages between the last 3 nucleotides of the guide nucleic acidsequence. In some embodiments, hybridization of the guide nucleic acid to the target sequence of the mutant allele activates cleavage of DNA, RNA, or a combination thereof in the cell, and induces cell cycle arrest, apoptosis, cell death, or a combination thereof, of the cell. In some embodiments, the effector protein selectively cleaves the mutant allele. In some embodiments, the effector protein does not cleave the wildtype allele. In some embodiments, the target gene is NRAS. In some embodiments, the mutant allele of the NRAS gene encodes a NRAS protein comprising the Q61L mutation. In some embodiments, the target sequence of the mutant allele comprises the sequence of SEQ ID NO: 94. In some embodiments, the spacer sequence is complementary to the target sequence comprising the sequence of SEQ ID NO: 94. In some embodiments, the spacer sequence is complementary to the target sequence that is adjacent to the PAM TCTA specific for the mutant allele. In some embodiments, the effector protein comprises a sequence that is at least 80%, at least 85%, at least 90%, at least 95%, at least 97%, at least 98%, at least 99%, or 100% identical to the sequence of SEQ ID NO: 1 or SEQ ID NO: 24. In some embodiments, the spacer sequence comprises a sequence that is at least 80%, at least 85%, at least 90%, at least 95%, or 100% identical to a sequence selected from SEQ ID NOs: 72-75. In some embodiments, the guide nucleic acid comprises a repeat sequence comprising a sequence that is at least 80%, at least 85%, at least 90%, at least 95%, or 100% identical to a sequence selected from SEQ ID NOs: 81-88. In some embodiments, the guide nucleic acid comprises a sequence that is at least 80%, at least 85%, at least 90%, at least 95%, or 100% identical to a sequence selected from SEQ ID NOs: 99-102. In some embodiments, the guide nucleic acid comprises a sequence that is at least 80%, at least 85%, at least 90%, at least 95%, or 100% identical to a sequence selected from SEQ ID NOs: 111-113. In some embodiments, the effector protein comprises a sequence that is at least 80%, at least 85%, at least 90%, at least 95%, at least 97%, at least 98%, at least 99%, or 100% identical to the sequence of SEQ ID NO: 3. In some embodiments, the spacer sequence comprises a sequence that is at least 80%, at least 85%, at least 90%, at least 95%, at least 97%, at least 98%, at least 99%, or 100% identical to a sequence selected from SEQ ID NOs: 77-78. In some embodiments, the guide nucleic acid comprises a repeatg sequence comprising a sequence that is at least 80%, at least 85%, at least 90%, at least 95%, or 100% identical to the sequence of SEQ ID NO: 89. In some embodiments, the guide nucleic acid comprises a sequence that is at least 80%, at least 85%, at least 90%, at least 95%, or 100% identical to a sequence SEQ ID NO: 104-105. In some embodiments, the guide nucleic acid comprises a sequence that is at least 80%, at least 85%, at least 90%, at least 95%, or 100% identical to a sequence selected from SEQ ID NO: 115-116.

In some aspects, disclosed herein are methods for SNP-specific editing of a target gene in a cell, comprising contacting the cell with an effector protein or a polynucleotide encoding the same, and a guide nucleic acid or a polynucleotide encoding the same, wherein the effector protein comprises a CasM.265466 or a variant thereof, and wherein the guide nucleic acid comprises a sequence that is complementary to a target sequence comprising a single nucleotide polymorphism (SNP) in the target gene. In some embodiments, the target gene is KRAS. In some embodiments, wherein the SNP is 35G>A in the KRAS gene encoding a KRAS protein comprising the G12D mutation. In some embodiments, the effector protein comprises the sequence of SEQ ID NO: 3. In some embodiments, wherein the guide nucleic acid comprises a modification of at least one nucleotide or internucleotide linkage. In some embodiments, the modification is selected from a 2′ O-methyl, a 2′-fluoro, a locked nucleic acid (LNA), a peptide nucleic acid (PNA), a phosphorothioate linkage, and a 5′ cap, and a combination thereof. In some embodiments, the modification comprises 2′ O-Methyl modifications of the first 3 nucleotides and last 3 nucleotides of the guide nucleic acidsequence, phosphorothioate linkages between the first 4 nucleotides of the guide nucleic acidsequence, and phosphorothioate linkages between the last 3 nucleotides of the guide nucleic acidsequence. In some embodiments, hybridization of the guide nucleic acid to the target sequence comprising the SNP activates cleavage of DNA, RNA, or a combination thereof in the cell, and induces cell cycle arrest, apoptosis, cell death, or a combination thereof, of the cell. In some embodiments, the spacer sequence comprises a sequence that is at least 80%, at least 85%, at least 90%, at least 95%, at least 97%, at least 98%, at least 99%, or 100% identical to the sequence of SEQ ID NO: 80. In some embodiments, the guide nucleic acid comprises a repeat sequence comprising a sequence that is at least 80%, at least 85%, at least 90%, at least 95%, or 100% identical to the sequence of SEQ ID NO: 89. In some embodiments, the guide nucleic acid comprises a sequence that is at least 80%, at least 85%, at least 90%, at least 95%, or 100% identical to the sequence of SEQ ID NO: 107. In some embodiments, the guide nucleic acid comprises a sequence that is at least 80%, at least 85%, at least 90%, at least 95%, or 100% identical to the sequence of SEQ ID NO: 118.

In some aspects, disclosed herein are guide nucleic acids or polynucleotides encoding the same, wherein the guide nucleic acid comprises (a) a first region comprising a repeat sequence, and (b) a second region comprising a spacer sequence that is complementary to a target sequence of a mutant allele of a NRAS gene, wherein the repeat sequence is capable of being bound by a clustered regularly interspaced short palindromic repeats (CRISPR) Cas protein other than a Cas9 protein. In some embodiments, the target sequence comprises at least a portion of the mutant allele of the NRAS gene. In some embodiments, the mutant allele of the NRAS gene encodes a NRAS protein comprising the Q61L mutation. In some embodiments, the guide nucleic acid comprises a modification of at least one nucleotide or internucleotide linkage. In some embodiments, the modification is selected from a 2′ O-methyl, a 2′-fluoro, a locked nucleic acid (LNA), a peptide nucleic acid (PNA), a phosphorothioate linkage, and a 5′ cap, and a combination thereof. In some embodiments, the modification comprises 2′ O-Methyl modifications of the first 3 nucleotides and last 3 nucleotides of the guide nucleic acid sequence, phosphorothioate linkages between the first 4 nucleotides of the guide nucleic acid sequence, and phosphorothioate linkages between the last 3 nucleotides of the guide nucleic acid sequence. In some embodiments, the Cas protein is at least 80%, at least 85%, at least 90%, at least 95% or 100% identical to a sequence selected from SEQ ID NOs: 1-2, and 24-43. In some embodiments, the spacer sequence is at least 80%, at least 85%, at least 90%, at least 95%, or 100% identical to a sequence selected from SEQ ID NOs: 72-75. In some embodiments, the repeat sequence is at least 80%, at least 85%, at least 90%, at least 95% or 100% identical to SEQ ID NOs: 81-88. In some embodiments, the guide nucleic acid comprises a sequence that is at least 80%, at least 85%, at least 90%, at least 95%, or 100% identical to a sequence selected from SEQ ID NOs: 99-102. In some embodiments, the guide nucleic acid comprises a sequence that is at least 80%, at least 85%, at least 90%, at least 95%, or 100% identical to a sequence selected from SEQ ID NOs: 111-113. In some embodiments, the Cas protein is at least 80%, at least 85%, at least 90%, at least 95% or 100% identical to a sequence selected from SEQ ID NOs: 3-4 and 44-61. In some embodiments, the spacer sequence is at least 80%, at least 85%, at least 90%, at least 95%, or 100% identical to a sequence selected from SEQ ID NOs: 77-78. In some embodiments, the repeat sequence is at least 80%, at least 85%, at least 90%, at least 95% or 100% identical to the sequence of SEQ ID NO: 89. In some embodiments, the guide nucleic acid comprises a sequence that is at least 80%, at least 85%, at least 90%, at least 95%, or 100% identical to a sequence selected from SEQ ID NOs: 104-105, 115 and 116.

In some aspects, disclosed herein are guide nucleic acids or polynucleotides encoding the same, wherein the guide nucleic acid comprises: (a) a first region comprising a repeat sequence, and (b) a second region comprising a spacer sequence that is complementary to a target sequence comprising a SNP in a KRAS gene, wherein the repeat sequence is capable of being bound by a clustered regularly interspaced short palindromic repeats (CRISPR) Cas protein other than a Cas9 protein. In some embodiments, the SNP is 35G>A in the KRAS gene encoding a KRAS protein comprising the G12D mutation. In some embodiments, the spacer sequence is at least 80%, at least 85%, at least 90%, at least 95%, or 100% identical to the sequence of SEQ ID NO: 80. In some embodiments, the repeat sequence is at least 80%, at least 85%, at least 90%, at least 95% or 100% identical to the sequence of SEQ ID NO: 89. In some embodiments, the guide nucleic acid comprises a sequence that is at least 80%, at least 85%, at least 90%, at least 95%, or 100% identical to the sequence of SEQ ID NOs: 107or 118. In some embodiments, the Cas protein is at least 80%, at least 85%, at least 90%, at least 95% or 100% identical to a sequence selected from SEQ ID NOs: 3-4 and 44-61.

In some aspects, disclosed herein are systems comprising the guide nucleic acid of any of the above aspects or embodiments, or a first polynucleotide encoding the same. In some embodiments, the systems comprise a Cas protein or a second polynucleotide encoding the same. In some embodiments, the first and/or second polynucleotide is an mRNA polynucleotide. In some embodiments, the first and/or second polynucleotide is a DNA expression vector. In some embodiments, the DNA expression vector is an adeno-associated viral (AAV) vector. In some embodiments, the systems comprise a recombinant adeno-associated virus (AAV) expression cassette comprising sequences encoding: a) a first inverted terminal repeat (ITR) and a first promoter; b) the Cas protein; c) optionally a second promoter; d) a second polynucleotide encoding the guide nucleic acid described herein; and e) a second ITR, wherein the AAV expression cassette is a self-complementary AAV vector. In some embodiments, the Cas protein recognizes a protospacer motif (PAM) of 5′-TNTN-3′ or 5′-TTN-3′. In some embodiments, the Cas protein recognizes the PAM sequence selected from the group consisting of 5′-TCTA-3′, 5′-TTTG-3′, 5′-TCTG-3′, 5′-TGTG-3′, 5′-TATA-3′, 5′-TTTA-3′, 5′-TGTA-3′, and 5′-TATG-3′. In some embodiments, the Cas protein comprises a sequence that is at least 80%, at least 85%, at least 90%, at least 95%, at least 97%, at least 98%, at least 99%, or 100% identical to a sequence selected from SEQ ID NOs: 3-4 and 44-61. In some embodiments, the Cas protein recognizes the PAM sequence selected from the group consisting of 5′-TTG-3′, 5′-TTC-3′, 5′-TTT-3′, and 5′-TTA-3′. In some embodiments, the Cas protein comprises a sequence that is at least 80%, at least 85%, at least 90%, at least 95%, at least 97%, at least 98%, at least 99%, or 100% identical to a sequence selected from SEQ ID NOs: 1-2 and 24-43. In some embodiments, the Cas protein has a positively charged amino acid at position 26 of SEQ ID NO: 1. In some embodiments, the positively charged amino acid is selected from arginine, histidine and lysine. In some embodiments, the Cas protein amino acid sequence comprises a nuclear localization signal. In some embodiments, the Cas protein reduces expression of the NRAS or KRAS gene. In some embodiments, the Cas protein cleaves DNA, RNA, or a combination thereof in a cell, and induces cell cycle arrest, apoptosis, cell death, or a combination thereof, of the cell. In some embodiments, the Cas protein selectively cleaves the mutant allele. In some embodiments, the Cas protein does not cleave the wildtype allele.

In some aspects, disclosed herein are nucleic acid expression vectors that encode a guide nucleic acid, wherein the guide nucleic comprises at least one sequence that is at least 80%, at least 85%, at least 90%, at least 95% or 100% identical to a sequence selected from any one of SEQ ID NOs:68-92 and 95-118. In some embodiments, the nucleic acid expression vector is an adenoviral associated viral (AAV) vector. In some embodiments, the nucleic acid expression vector further comprises a polynucleotide encoding an effector protein that is at least 80%, at least 85%, at least 90%, at least 95%, at least 99%, or 100% identical to any one of the sequences recited in TABLE 1, TABLE 4, and TABLE 6.

In some aspects, provided herein are pharmaceutical compositions comprising the guide nucleic acid of any of the above aspects or embodiments, the system of any of the above aspects or embodiments, the nucleic acid expression vector of any of the above aspects or embodiments, and a pharmaceutical acceptable carrier.

In some aspects, provided herein are cells, or populations of cells, comprising or modified by the guide nucleic acid of any of the above aspects or embodiments, the system of any of the above aspects or embodiments, or the nucleic acid expression vector of any of the above aspects or embodiments.

In some aspects, provided herein are methods of modifying a NRAS gene, comprising contacting the NRAS gene with the guide nucleic acid of any of the above aspects or embodiments, the system of any of the above aspects or embodiments, or the nucleic acid expression vector of any of the above aspects or embodiments. In some embodiments, the modifying of the NRAS gene comprises cleaving the NRAS gene, deleting a nucleotide of the NRAS gene, inserting a nucleotide into the NRAS gene, substituting a nucleotide of the NRAS gene with an alternative nucleotide, or editing a nucleotide, more than one of the foregoing, or any combination thereof. In some embodiments, the modifying of the NRAS gene comprises inducing cell cycle arrest, apoptosis, cell death, or a combination thereof, in a cell. In some embodiments, the method is performed in a cell. In some embodiments, the method is performed in vivo.

In some aspects, provided herein are methods of modifying a KRAS gene, comprising contacting the KRAS gene with the guide nucleic acid of any of the above aspects or embodiments, the system of any of the above aspects or embodiments, or the nucleic acid expression vector of any of the above aspects or embodiments. In some embodiments, the modifying of the KRAS gene comprises cleaving the KRAS gene, deleting a nucleotide of the KRAS gene, inserting a nucleotide into the KRAS gene, substituting a nucleotide of the KRAS gene with an alternative nucleotide, or editing a nucleotide, more than one of the foregoing, or any combination thereof. In some embodiments, the modifying of the KRAS gene comprises inducing cell cycle arrest, apoptosis, cell death, or a combination thereof, in a cell. In some embodiments, the method is performed in a cell. In some embodiments, the method is performed in vivo.

In some aspects, provided herein are methods of treating a disease associated with expression of a mutated target gene, the method comprising contacting a cell that expresses the mutated target gene with the guide nucleic acid of any of the above aspects or embodiments, the system of any of the above aspects or embodiments, or the nucleic acid expression vector of any of the above aspects or embodiments. In some embodiments, he mutated target gene encodes a NRAS protein comprising the Q61L mutation. In some embodiments, the methods comprise modifying the mutant allele of the NRAS gene. In some embodiments, modifying the mutant allele of the NRAS gene comprises selectively cleaving the mutant allele without cleaving a wildtype allele. In some embodiments, the disease is a cancer. In some embodiments, the cancer is selected from hepatocellular carcinoma, melanoma, leukemia, skin cancer, colorectal cancer, acute myeloid leukemia (AML), thyroid cancer, liver cancer, lung cancer, neuroblastoma, bladder cancer, and rhabdomyosarcoma. In some embodiments, the mutated target gene encodes a KRAS protein comprising the G12D mutation. In some embodiments, the mutated target gene comprises a SNP. In some embodiments, the SNP is 35G>A. In some embodiments, the disease is a cancer. In some embodiments, the cancer is selected from pancreatic cancer, colon cancer, and lung cancer.

In some aspects, provided herein are methods of killing a cancer cell comprising contacting the cell with the guide nucleic acid of any of the above aspects or embodiments, the system of any of the above aspects or embodiments, or the nucleic acid expression vector of any of the above aspects or embodiments. In some embodiments, the cell is in vivo. In some embodiments, the cell is in vitro.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows the percent indel formation by CasPhi12, CasPhi12 L26R or CasM.265466 with guides targeting the wildtype or mutant allele of the NRAS gene in the control Hep3B cells.

FIG. 2A-FIG. 2B show the percent indel formation by CasPhi12, CasPhi12 L26R or CasM.265466 with guides targeting the wildtype or mutant allele of the NRAS gene in the heterozygous Hep2G cells. FIG. 2A shows the total percent indel formation induced by each guide and FIG. 2B shows the percent indel formation in the wildtype or mutant allele induced by each guide.

FIG. 3A-FIG. 3B shows the colony counts of the Hep2G cells after transfected with nuclease mRNA and guide nucleic acid. FIG. 3A shows the colony counts of the Hep2G cells transfected nuclease mRNA encoding CasPhi12 or CasPhi12 L26R. FIG. 3B shows the colony counts of the Hep2G cells transfected with mRNA encoding CasM.265466.

FIG. 4A-FIG. 4C show the percent indel formation by CasPhi12 L26R paired with a guide nucleic acid comprising a repeat sequence having 36, 30 or 24 nucleotides and a spacer sequence having 20, 19, 18, or 17 nucleotides. FIG. 4A shows the total percent indel formation induced by each guide in the HepG2 cells; FIG. 4B shows the percent indel formation in the wildtype or mutant allele induced by each guide in the HepG2 cells; and FIG. 4C shows the total percent indel formation induced by each guide in the Hep3B cells.

FIG. 5A-FIG. 5B show the percent indel formation by CasPhi12 L26R with modified guides targeting the wildtype or mutant allele of the NRAS gene. FIG. 5A shows the percent indel formation in Hep3B cells; FIG. 5B shows the total percent indel formation induced in the HepG2 cells; and FIG. 5C shows the percent indel formation in the wildtype or mutant allele in the HepG2 cells.

FIG. 6A-FIG. 6B show the percent indel formation by CasPhi12 L26R with the modified guides R13990-M6 targeting the wildtype allele or R14002-M6 targeting the mutant allele of the NRAS gene. FIG. 6A shows the percent indel formation in Hep3B cells; FIG. 6B shows the total percent indel formation induced in the HepG2 cells; and FIG. 6C shows the percent indel formation in the wildtype or mutant allele in the HepG2 cells.

FIG. 7A-FIG. 7C show the percent indel formation by CasM.265466 with the modified guides R13833 targeting the wildtype allele or R13834 targeting the mutant allele of the NRAS gene. FIG. 7A shows the percent indel formation in Hep3B cells; FIG. 7B shows the total percent indel formation induced in the HepG2 cells; and FIG. 7C shows the percent indel formation in the wildtype or mutant allele in the HepG2 cells.

FIG. 8A-FIG. 8B show the percent indel formation by CasM.265466, CasPhi12, or Cas9 with guides targeting the wildtype or mutant allele of the KRAS gene. FIG. 8A shows the percent indel formation induced by each Cas nuclease in AsPC1 cells and FIG. 8B shows the percent indel formation induced by each Cas nuclease in BxPC-3 cells.

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### 1. Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any, and all, combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The terms, “% identical,” “% identity,” and “percent identity,” or grammatical equivalents thereof, refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG.

The term “base editing enzyme,” as used herein, refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

The term “base editor,” as used herein, refers to a fusion protein comprising a base editing enzyme fused or linked to an effector protein. The base editing enzyme may be referred to as a fusion partner. The base editing enzyme can differ from a naturally occurring base editing enzyme. It is understood that any reference to a base editing enzyme herein also refers to a base editing enzyme variant. The base editor is functional when the effector protein is coupled to a guide nucleic acid. The guide nucleic acid imparts sequence specific activity to the base editor. By way of non-limiting example, the effector protein may comprise a catalytically inactive effector protein. Also, by way of non-limiting example, the base editing enzyme may comprise deaminase activity. Additional base editors are described herein.

The term “catalytically inactive effector protein,” also referred to as a “dCas” protein, as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein. In some embodiments, the catalytically inactive effector protein is referred to as a dead Cas protein or a dCas protein.

The term “cis cleavage,” as used herein, refers to cleavage (hydrolysis of a phosphodiester bond) of a target nucleic acid by an effector protein complexed with a guide nucleic acid (e.g., an RNP complex), wherein at least a portion of the guide nucleic acid is hybridized to at least a portion of the target nucleic acid. Cleavage may occur within or directly adjacent to the region of the target nucleic acid that is hybridized to the guide nucleic acid.

The terms “complementary” and “complementarity,” as used herein, with reference to a nucleic acid molecule or nucleotide sequence, refer to the characteristic of a polynucleotide having nucleotides that base pair with their Watson-Crick counterparts (C with G; or A with T or U) in a reference nucleic acid. For example, when every nucleotide in a polynucleotide forms a base pair with a reference nucleic acid, that polynucleotide is said to be 100% complementary to the reference nucleic acid. In a double stranded DNA or RNA sequence, the upper (sense) strand sequence is in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the ‘reverse complement’ sequence or the ‘reverse complementary’ sequence is understood as the sequence of the lower strand in the direction of its 5′- to its 3′-end. Each nucleotide in a double stranded DNA or RNA molecule that is paired with its Watson-Crick counterpart called its complementary nucleotide.

The term “cleavage assay,” as used herein, refers to an assay designed to visualize, quantitate, or identify cleavage of a nucleic acid. In some cases, the cleavage activity may be cis-cleavage activity. In some cases, the cleavage activity may be trans-cleavage activity.

The terms “cleave,” “cleaving,” and “cleavage,” as used herein, with reference to a nucleic acid molecule or nuclease activity of an effector protein, refer to the hydrolysis of a phosphodiester bond of a nucleic acid molecule that results in breakage of that bond. The result of this breakage can be a nick (hydrolysis of a single phosphodiester bond on one side of a double-stranded molecule), single strand break (hydrolysis of a single phosphodiester bond on a single-stranded molecule) or double strand break (hydrolysis of two phosphodiester bonds on both sides of a double-stranded molecule) depending upon whether the nucleic acid molecule is single-stranded (e.g., ssDNA or ssRNA) or double-stranded (e.g., dsDNA) and the type of nuclease activity being catalyzed by the effector protein.

The term “clustered regularly interspaced short palindromic repeats (CRISPR),” as used herein, refers to a segment of DNA found in the genomes of certain prokaryotic organisms, including some bacteria and archaea, that includes repeated short sequences of nucleotides interspersed at regular intervals between unique sequences of nucleotides derived from the DNA of a pathogen (e.g., virus) that had previously infected the organism and that functions to protect the organism against future infections by the same pathogen.

The terms “CRISPR RNA” or “crRNA,” as used herein, refer to a type of guide nucleic acid, wherein the nucleic acid is RNA comprising a first sequence that is capable of interacting with an effector protein either directly (by being bound by an effector protein) or indirectly (e.g., by hybridization with a second nucleic acid molecule that can be bound by an effector, such as a tracrRNA); and a second sequence that hybridizes to a target sequence of a target nucleic acid. In some embodiments, the first sequence is referred to as a repeat sequence and the second sequence is referred to as a spacer sequence. The first sequence and the second sequence are directly connected to each other or by a linker.

The term “detectable signal,” as used herein, refers to a signal that can be detected using optical, fluorescent, chemiluminescent, electrochemical and other detection methods known in the art.

The term, “disrupt,” as used herein, refers to reducing or abolishing a function of a gene regulatory element by altering or modifying the nucleotide sequence of the gene regulatory element or the nucleotide sequence located in proximity (e.g., less than 200 linked nucleotides) to the gene regulatory element. In some embodiments, the gene regulatory element is a splicing-regulatory element. In some embodiments, the original function of the gene regulatory element is repressing exonic splicing. In some embodiments, there is an increased inclusion of an exon region in a mature mRNA after the disruption.

The term, “donor nucleic acid,” as used herein, refers to a nucleic acid that is (designed or intended to be) incorporated into a target nucleic acid or target sequence.

The term “dual nucleic acid system” as used herein refers to a system that uses a transactivated or transactivating RNA-crRNA duplex complexed with one or more polypeptides described herein, wherein the complex is capable of interacting with a target nucleic acid in a sequence selective manner.

The term “effector protein,” as used herein, refers to a protein, polypeptide, or peptide that is capable of interacting with a guide nucleic acid to form a complex (e.g., a RNP complex), wherein the complex interacts with a target nucleic acid. A complex between an effector protein and a guide nucleic acid can include multiple effector proteins or a single effector protein. In some embodiments, the effector protein modifies the target nucleic acid when the complex contacts the target nucleic acid. In some embodiments, the effector protein does not modify the target nucleic acid, but it is linked to a fusion partner protein that modifies the target nucleic acid when the complex contacts the target nucleic acid. A non-limiting example of an effector protein modifying a target nucleic acid is cleaving of a phosphodiester bond of the target nucleic acid. Additional examples of modifications an effector protein can make to target nucleic acids are described herein and throughout. Herein, reference to an effector protein includes reference to a nucleic acid encoding the effector protein, unless indicated otherwise.

The term, “engineered modification,” as used herein, refers to a structural change of one or more nucleic acid residues of a nucleotide sequence or one or more amino acid residue of an amino acid sequence, such as chemical modification of one or more nucleobases; or a chemical change to the phosphate backbone, a nucleotide, a nucleobase, or a nucleoside. Such modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition, or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some embodiments, the nucleic acids provided herein are not uniformly modified along the entire length of the molecule. Different nucleotide modifications and/or backbone structures can exist at various positions within the nucleic acid.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

The terms “fusion protein,” or “fusion effector protein,” as used herein, refer to a protein comprising at least two heterologous polypeptides. The fusion protein may comprise one or more effector proteins and fusion partners. In some embodiments, an effector protein and fusion partner are not found connected to one another as a native protein or complex that occurs together in nature.

The term “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modification, nucleic acid cleavage, protein binding. The absence of the functional domain, including mutations of the functional domain, would abolish or reduce activity.

The term, “genetic disease,” as used herein, refers to a disease, disorder, condition, or syndrome associated with or caused by one or more mutations in the DNA of an organism having the genetic disease.

The term “guide nucleic acid,” as used herein, refers to a nucleic acid comprising: a first nucleotide sequence that is capable of being non-covalently bound by an effector protein; and a second nucleotide sequence that hybridizes to a target nucleic acid. When in a complex with one or more polypeptides described herein (e.g., an RNP complex), a guide nucleic acid can impart sequence selectivity to the complex when the complex interacts with a target nucleic acid. The first sequence may be referred to herein as a repeat sequence. The second sequence may be referred to herein as a spacer sequence. The term, “guide nucleic acid,” may be used interchangeably herein with the term “guide RNA” (gRNA) however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary RNA sequence, that is capable of being non-covalently bound by an effector protein. In some embodiments, the handle sequence further comprises a repeat sequence. In such embodiments, the intermediary RNA sequence or a combination of the intermediary RNA and the repeat sequence is capable of being non-covalently bound by an effector protein.

The term “heterologous,” as used herein, means a nucleotide or polypeptide sequence that is not found in a native nucleic acid or protein, respectively. In some embodiments, fusion proteins comprise an effector protein and a fusion partner protein, wherein the fusion partner protein is heterologous to an effector protein. These fusion proteins may be referred to as a “heterologous protein.” A protein that is heterologous to the effector protein is a protein that is not covalently linked via an amide bond to the effector protein in nature. In some embodiments, a heterologous protein is not encoded by a species that encodes the effector protein. In some embodiments, the heterologous protein exhibits an activity (e.g., enzymatic activity) when it is linked to the effector protein. In some embodiments, the heterologous protein exhibits increased or reduced activity (e.g., enzymatic activity) when it is linked to the effector protein, relative to when it is not linked to the effector protein. In some embodiments, the heterologous protein exhibits an activity (e.g., enzymatic activity) that it does not exhibit when it is linked to the effector protein. A guide nucleic acid may comprise a first sequence and a second sequence, wherein the first sequence and the second sequence are not found covalently linked via a phosphodiester bond in nature. Thus, the first sequence is considered to be heterologous with the second sequence, and the guide nucleic acid may be referred to as a heterologous guide nucleic acid.

The terms, “intermediary RNA,” “intermediary RNA sequence,” and “intermediary sequence” as used herein, in a context of a single nucleic acid system, refers to a nucleotide sequence in a handle sequence, wherein the intermediary RNA sequence is capable of, at least partially, being non-covalently bound to an effector protein to form a complex (e.g., an RNP complex). An intermediary RNA sequence is not a transactivating nucleic acid in systems, methods, and compositions described herein.

The term “linked” when used in reference to biopolymers (e.g., nucleic acids, polypeptides) refers to being covalently connected. In some embodiments, two polymers are linked by at least a covalent bond. In some embodiments, two nucleic acids are linked by at least one nucleotide. In some embodiments, two nucleic acids are linked by at least one amino acid. The terms “fused” and “linked” are used interchangeably herein.

The term “linker,” as used herein, refers to a covalent bond or molecule that links a first polypeptide to a second polypeptide (e.g., by an amide bond, or one or more amino acids) or a first nucleic acid to a second nucleic acid (e.g., by a phosphodiester bond, or one or more nucleotides).

The term “modified target nucleic acid,” as used herein, refers to a target nucleic acid, wherein the target nucleic acid has undergone a modification, for example, after contact with an effector protein. In some cases, the modification is an alteration in the sequence of the target nucleic acid. In some cases, the modified target nucleic acid comprises an insertion, deletion, or replacement of one or more nucleotides compared to the unmodified target nucleic acid.

The terms “non-naturally occurring” and “engineered,” as used herein, are used interchangeably and indicate the involvement of the hand of man. The terms, when referring to a nucleic acid, nucleotide, protein, polypeptide, peptide or amino acid, refer to a nucleic acid, nucleotide, protein, polypeptide, peptide or amino acid that is at least substantially free from at least one other feature with which it is naturally associated in nature and as found in nature, and/or contains a modification (e.g., chemical modification, nucleotide sequence, or amino acid sequence) that is not present in the naturally occurring nucleic acid, nucleotide, protein, polypeptide, peptide, or amino acid. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The term “nucleic acid expression vector,” as used herein, refers to a nucleic acid that can be used to express a nucleic acid of interest.

The term “nuclear localization signal (NLS),” as used herein, refers to an entity (e.g., peptide) that facilitates localization of a nucleic acid, protein, or small molecule to the nucleus, when present in a cell that contains a nuclear compartment.

The term “nuclease activity,” as used herein, refers to the catalytic activity that results in nucleic acid cleavage (e.g., ribonuclease activity (ribonucleic acid cleavage), or deoxyribonuclease activity (deoxyribonucleic acid cleavage), etc.).

The terms “partner protein,” “fusion partner,” or “fusion partner protein” as used herein, refer to a protein, polypeptide or peptide that is linked to an effector protein or capable of being proximal to an effector protein. In some embodiments, a fusion partner that is capable of being proximal to an effector protein is a fusion partner that is capable of binding a guide nucleic acid, wherein the effector protein is also capable of binding the guide nucleic acid. In some embodiments, a fusion partner directly interacts with (e.g., binds to/by) an effector protein. In some embodiments, a fusion partner indirectly interacts with an effector protein (e.g., through another protein or moiety).

The term “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by well-known conventional methods (see, e.g., Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005).

The terms “protospacer adjacent motif” and “PAM” as used herein, refer to a nucleotide sequence found in a target nucleic acid that directs an effector protein to modify the target nucleic acid at a specific location. In some embodiments, a PAM sequence is required for a complex of an effector protein and a guide nucleic acid (e.g., an RNP complex) to hybridize to and edit the target nucleic acid. In some embodiments, the complex does not require a PAM to edit the target nucleic acid.

In some embodiments, the term “region” as used herein may be used to describe a portion of, or all of, a corresponding sequence, for example, a spacer region is understood to comprise a portion of or all of a spacer sequence.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat sequence,” as used herein, refers to a sequence of nucleotides in a guide nucleic acid that is capable of, at least partially, interacting with an effector protein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The terms, “RuvC” and “RuvC domain,” as used herein, refer to a region of an effector protein that is capable of cleaving a target nucleic acid, and in certain embodiments, of processing a pre-crRNA. In some embodiments, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as InterPro (ebi.ac.uk/interpro/). For example, a RuvC-like domain may be a domain which shares homology with a region of TnpB proteins of the IS605 and other related families of transposons

The term “sample,” as used herein, generally refers to something comprising a target nucleic acid. In some embodiments, the sample is a biological sample, such as a biological fluid or tissue sample. In some embodiments, the sample is an environmental sample. The sample may be a biological sample or environmental sample that is modified or manipulated. By way of non-limiting example, samples may be modified or manipulated with purification techniques, heat, nucleic acid amplification, salts and buffers.

The terms, “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some embodiments activating the effector protein, and hybridizing to a target nucleic acid, without the need for a second nucleic acid molecule). For example, an sgRNA can have two or more linked guide nucleic acid components (e.g., an intermediary RNA sequence, a repeat sequence, a spacer sequence and optionally a linker). In some embodiments, a sgRNA comprises a handle sequence, wherein the handle sequence comprises an intermediary sequence, a repeat sequence, and optionally a linker sequence.

The term, “single nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein.

The term, “spacer sequence,” as used herein, refers to a nucleotide sequence in a guide nucleic acid that is capable of, at least partially, hybridizing to an equal length portion of a sequence (e.g., a target sequence) of a target nucleic acid.

The term “subject,” as used herein, refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a non-human primate. The mammal can be a cynomolgus monkey. The mammal can be a mouse, rat, or other rodent. The mammal can be a human. The subject may be diagnosed or suspected of being at high risk for a disease. In some embodiments, the subject is not necessarily diagnosed or suspected of being at high risk for the disease.

The term “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The terms “target nucleic acid sequence” and “target sequence,” as used herein, when used in reference to a target nucleic acid, refers to a sequence of nucleotides found within a target nucleic acid. Such a sequence of nucleotides can, for example, hybridize to an equal length portion of a guide nucleic acid. Hybridization of the guide nucleic acid to the target sequence may bring an effector protein into contact with the target nucleic acid.

The term, “trans cleavage,” as used herein, in the context of cleavage (e.g., hydrolysis of a phosphodiester bond) of one or more target nucleic acids or non-target nucleic acids, or both, by an effector protein that is complexed with a guide nucleic acid and the target nucleic acid. Trans cleavage activity may be triggered by the hybridization of a guide nucleic acid to a target nucleic acid. The effector may cleave a target strand as well as non-target strand, wherein the target nucleic is a double stranded nucleic acid. Trans cleavage of the target nucleic acid may occur away from (e.g., not within or directly adjacent to) the portion of the target nucleic acid that is hybridized to the portion of the guide nucleic acid.

The terms, “transactivating”, “trans-activating”, “trans-activated”, “transactivated” and grammatical equivalents thereof, as used herein, in the context of a dual nucleic acid system refers to an outcome of the system, wherein a polypeptide is enabled to have a binding and/or nuclease activity on a target nucleic acid by a tracrRNA-crRNA duplex.

The terms, “trans-activating RNA”, “transactivating RNA” and “tracrRNA,” refer to a transactivating or transactivated nucleic acid in a dual nucleic acid system that is capable of hybridizing, at least partially, to a crRNA to form a tracrRNA-crRNA duplex, and of interacting with an effector protein to form a complex (e.g., an RNP complex).

The term, “transcriptional activator,” as used herein, refers to a polypeptide or a fragment thereof that can activate or increase transcription of a target nucleic acid molecule.

The term “transcriptional repressor,” as used herein, refers to a polypeptide or a fragment thereof that is capable of arresting, preventing, or reducing transcription of a target nucleic acid.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. A donor nucleic acid can comprise a transgene. The cell in which transgene expression occurs can be a target cell, such as a host cell.

The terms “treatment” and “treating,” as used herein, are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “viral vector,” as used herein, refers to a nucleic acid to be delivered into a host cell via a recombinantly produced virus or viral particle. The nucleic acid may be single-stranded or double stranded, linear or circular, segmented or non-segmented. The nucleic acid may comprise DNA, RNA, or a combination thereof. Non-limiting examples of viruses or viral particles that can deliver a viral vector include retroviruses (e.g., lentiviruses and 7-retroviruses), adenoviruses, arenaviruses, alphaviruses, adeno-associated viruses (AAVs), baculoviruses, vaccinia viruses, herpes simplex viruses and poxviruses. A viral vector delivered by such viruses or viral particles may be referred to by the type of virus to deliver the viral vector (e.g., an AAV viral vector is a viral vector that is to be delivered by an adeno-associated virus). A viral vector referred to by the type of virus to be delivered by the viral vector can contain viral elements (e.g., nucleotide sequences) necessary for packaging of the viral vector into the virus or viral particle, replicating the virus, or other desired viral activities. A virus containing a viral vector may be replication competent, replication deficient or replication defective.

The term “mutation,” as used herein, refers to a change in the nucleotide sequence of a gene that may be caused by deletion, insertion or substitution of one or more nucleotides in the gene. A cancer-associated mutation refers to a mutation that is present in the cell of an individual who has cancer.

The term “allele,” as used herein, refers to one version of a nucleotide sequence at a given genetic locus. Each genetic locus has two alleles. If the two alleles are the same, an individual is homozygous for that locus. If the two alleles are different, the individual is heterozygous for locus.

The term “wildtype allele,” as used herein, refers to a non-mutated version of a nucleotide sequence at a given genetic locus. The term “mutant allele,” as used herein, refers to a version of a nucleotide sequence at a given genetic locus that contains one or more mutations relative to a corresponding wildtype allele.

The term “allele-specific editing,” as used herein, refers to editing of one allele of a genetic locus by the systems and composition described herein, while leaving the second allele of the genetic locus un-edited. Editing may comprise one or more of: cleaving the nucleotide sequence, deleting one or more nucleotides, inserting one or more nucleotides, mutating one or more nucleotides, or modifying (e.g., methylating, demethylating, deaminating, or oxidizing) of one or more nucleotides.

The term “single nucleotide polymorphism (SNP),” as used herein, refers to a single nucleotide difference at a specific location in the DNA sequence among individuals in a population, where each variation is present to some appreciable degree within the population (e.g., >1%). A subset of SNPs gives rise to changes in the encoded amino acid sequence.

The term “SNP-specific editing,” as used herein, refers to editing of a DNA sequence comprising a specific SNP by the systems and composition described herein. In some embodiments, the specific SNP gives rise to changes in the encoded amino acid sequence. Editing may comprise one or more of: cleaving the nucleotide sequence, deleting one or more nucleotides, inserting one or more nucleotides, mutating one or more nucleotides, or modifying (e.g., methylating, demethylating, deaminating, or oxidizing) of one or more nucleotides.

### 2. Introduction

Disclosed herein are compositions, systems and methods for detection and/or editing of a target nucleic acid (e.g., KRAS or NRAS). In some embodiments, the present disclosure provides guide nucleic acids that are capable of binding to a target sequence in a target gene (or mutated versions thereof). In some embodiments, the present disclosure provides guide nucleic acids that are capable of binding to a target sequence in a target gene (or mutated versions thereof) and an effector protein. In some embodiments, the effector protein is a programmable CRISPR-associated (Cas) protein. In general, Cas proteins bind and/or modify nucleic acids in a sequence-specific manner. Cas proteins with guide nucleic acids may modify DNA at a precise target location in the genome of a wide variety of cells and organisms, allowing for precise and efficient editing of DNA sequences of interest and can be used as an effective means to disrupt a gene of interest, generate DNA or RNA modifications, or to kill cells that express this mutation. In some embodiments, compositions and methods disclosed herein are useful for modifying a first allele of a target gene (e.g., a mutant allele), and do not modify a second allele of a target gene (e.g., a wildtype allele). In some embodiments, the present disclosure provides methods for treating a disease (e.g., cancer) by disrupting one or more target genes.

Disclosed herein are non-naturally occurring compositions and systems comprising an effector protein and/or a guide nucleic acid. In general, an effector protein and a guide nucleic acid refer to an effector protein and a guide nucleic acid, respectively, that are not found in nature. In some embodiments, systems and compositions herein comprise at least one non-naturally occurring component. For example, compositions and systems may comprise a guide nucleic acid, wherein the sequence of the guide nucleic acid is different or modified from that of a naturally-occurring guide nucleic acid. In some embodiments, compositions and systems comprise at least two components that do not naturally occur together. For example, compositions and systems may comprise a guide nucleic acid comprising a repeat sequence and a spacer sequence which do not naturally occur together. Also, by way of example, composition and systems may comprise a guide nucleic acid and an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the present disclosure provides compositions, systems, and methods for detecting and/or editing the NRAS gene or a mutated version thereof. The NRAS gene encodes the neuroblastoma RAS viral (v-ras) oncogene homolog (NRAS). In some embodiments, compositions and methods disclosed herein modify a mutant allele of a NRAS gene, and do not modify a wildtype allele of a NRAS gene. Such embodiments are referred to herein as “allele-specific” editing. In some embodiments, the mutant and wild type NRAS alleles are present in the same cell (e.g., the cell is heterozygous for the NRAS mutant).

In some embodiments, the present disclosure provides compositions, systems, and methods for detecting and/or editing the KRAS gene or a mutated version thereof. The KRAS gene encodes the Kirsten rat sarcoma virus (KRAS). In some embodiments, compositions and methods disclosed herein modify a mutant allele of a KRAS gene, and do not modify a wildtype allele of a KRAS gene. In some embodiments, the mutant and wild type KRAS alleles are present in the same cell (e.g., the cell is heterozygous for the KRAS mutant).

### 3. Effector Proteins

In some embodiments, compositions provided herein comprise one or more effector proteins. In some embodiments, compositions and systems described herein comprise an effector protein that is similar to a naturally occurring effector protein. The effector protein may lack a portion of the naturally occurring effector protein. The effector protein may comprise a mutation relative to the naturally-occurring effector protein, wherein the mutation is not found in nature.

An effector protein may be brought into proximity of a target nucleic acid in the presence of a guide nucleic acid. The ability of an effector protein to modify a target nucleic acid may be dependent upon the effector protein being bound to a guide nucleic acid and the guide nucleic acid being hybridized to a target nucleic acid. An effector protein may also recognize a protospacer adjacent motif (PAM) sequence present in the target nucleic acid, which may direct the modification activity of the effector protein.

In some embodiments, the effector protein is a programmable nuclease (e.g., a CRISPR-associated (Cas) protein) that modifies a target sequence in a target nucleic acid. In some embodiments, the effector protein is a programmable nuclease that modifies a region of the nucleic acid that is near, but not within, to the target sequence. Effector proteins may cleave nucleic acids, including single stranded RNA (ssRNA), double stranded DNA (dsDNA), and single-stranded DNA (ssDNA). Effector proteins may provide cis cleavage activity, trans cleavage activity, nickase activity, or a combination thereof.

In some embodiments, the effector protein may also comprise at least one additional amino acid relative to the naturally-occurring effector protein. For example, the effector protein may comprise an addition of a nuclear localization signal relative to the natural occurring effector protein. In some embodiments compositions and systems described herein may comprise a nuclear localization signal (NLS). In some embodiments, the effector protein is linked to a nuclear localization signal. In some embodiments, compositions and systems described herein may comprise a NLS sequence that is adjacent to the N terminal of the effector protein or that is adjacent to the C terminal of the effector protein, or both. In some embodiments, a nuclear localization signal can comprise a sequence of N-MAPKKKRKVGIHGVPAA-C (SEQ ID NO: 62). In some embodiments, a nuclear localization signal can comprise a sequence of N-KRPAATKKAGQAKKKK-C (SEQ ID NO: 63). In certain embodiments, the nucleotide sequence encoding the effector protein is codon optimized (e.g., for expression in a eukaryotic cell) relative to the naturally occurring sequence.

TABLE 2 provides exemplary nuclear localization sequences.

An effector protein may function as a single protein that is capable of binding to a guide nucleic acid and modifying a target nucleic acid. Alternatively, an effector protein may function as part of a multiprotein complex, including, for example, a complex having two or more effector proteins, including two or more of the same effector proteins (e.g., a dimer or a multimer). An effector protein, when functioning in a multiprotein complex, may have only one functional activity (e.g., binding to a guide nucleic acid), while other effector proteins present in the multiprotein complex are capable of another functional activity (e.g., modifying a target nucleic acid).

In some embodiments, the effector protein is a Type V Cas protein. In some embodiments, the effector protein is CasM.265466 or a variant thereof. A CasM.265466 is around one third of the size of Cas9. The smaller size of CasM.265466 make it ideal to be packaged together with its corresponding guide RNAs into a single AAV vector, thus overcoming the drawbacks of dual AAV vector systems.

TABLE 1 provides illustrative amino acid sequences of effector proteins. In some embodiments, an effector protein is at least 65%, at least 70%, at least 75%, at least 80%, at least 85%, at least 90%, at least 95%, at least 97%, or at least 98%, at least 99%, or 100% identical to the sequence as set forth in TABLE 1. In TABLE 1, bold and italicized text indicates the NLS sequence. Underlined text indicates a 3xFLAG tag.

In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 98%, at least 99%, or 100% identical to any one of the sequences as set forth in TABLE 1.

In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 1. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 1, wherein the amino acid residue at position 26, relative to SEQ ID NO: 1, remains unchanged. In some embodiments, the amino acid sequence of the effector protein is at least 65%, at least 70%, at least 75%, at least 80%, at least 85%, at least 90%, at least 95%, at least 97%, or at least 98%, at least 99%, or 100% identical to any one of the sequences as set forth in TABLE 1, wherein the amino acid residue at position 220, relative to SEQ ID NO: 3, remains unchanged.

In certain embodiments, compositions comprise an effector protein and a guide nucleic acid, wherein the amino acid sequence of the effector protein comprises at least about 200, at least about 220, at least about 240, at least about 260, at least about 280, at least about 300, at least about 320, at least about 340, at least about 360, at least about 380, at least about 400, or at least 420,at least about 420, at least about 440, at least about 460, at least about 480, at least about 500, at least about 520, at least about 540, at least about 560, at least about 580, at least about 600, at least about 620, at least about 640, at least about 660, at least about 680, at least about 700, or at least about 717 contiguous amino acids or more of any one of the sequences as set forth in TABLE 1.

In certain embodiments, compositions comprise an effector protein and a guide nucleic acid, wherein the amino acid sequence of the effector protein comprises at least 200, at least 220, at least 240, at least 260, at least 280, at least 300, at least 320, at least 340, at least 360, at least 380, at least 400, or at least 420, contiguous amino acids or more of any one of the sequences as set forth in TABLE 1.

In certain embodiments, compositions comprise an effector protein and a guide nucleic acid, wherein the amino acid sequence of the effector protein comprises at least about 200 contiguous amino acids or more of any one of the sequences as set forth in TABLE 1. In certain embodiments, compositions comprise an effector protein and a guide nucleic acid, wherein the amino acid sequence of the effector protein comprises at least about 300 contiguous amino acids or more of any one of the sequences as set forth in TABLE 1. In certain embodiments, compositions comprise an effector protein and a guide nucleic acid, wherein the amino acid sequence of the effector protein comprises at least about 400 contiguous amino acids or more of any one of the sequences as set forth in TABLE 1.

In some embodiments, compositions, systems, and methods described herein comprise an effector protein or a nucleic acid encoding the effector protein, wherein the effector protein comprises one or more amino acid alterations relative to the sequence recited in TABLE 1. In some embodiments, the effector protein comprising one or more amino acid alterations is a variant of an effector protein described herein. It is understood that any reference to an effector protein herein also refers to an effector protein variant as described herein. In some embodiments, an amino acid alteration comprises a deletion of an amino acid. In some embodiments, an amino acid alteration comprises an insertion of an amino acid. In some embodiments, an amino acid alteration comprises a conservative amino acid substitution. In some embodiments, an amino acid alteration comprises a non-conservative amino acid substitution. In some embodiments, one or more amino acid alterations comprises a combination of one or more conservative amino acid substitutions and one or more non-conservative amino acid substitutions. When describing a conservative amino acid substitution herein, reference is made to the replacement of one amino acid for another such that the replacement takes place within a family of amino acids that are related in their side chains. Conversely, when describing a non-conservative alteration (e.g., non-conservative substitution), reference is made to the replacement of one amino acid residue for another that does not have a related side chain. It is understood that genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl. Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLE 1, wherein the effector protein comprises 1, 2, 3, 4, 5, 6, 7, 8, 9 or 10 conservative amino acid substitutions relative to the sequence selected from TABLE 1. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLE 1, wherein the effector protein comprises 1 to 10, 10 to 20, 20 to 30, or 30 to 40 conservative amino acid substitutions relative to the sequence selected from TABLE 1. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLE 1, wherein the effector protein comprises not more than 1, 2, 3, 4, 5, 6, 7, 8, 9 or 10 non-conservative amino acid substitutions relative to the sequence selected from TABLE 1.

In certain embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 98%, at least 99%, or 100% similar to any one of the sequences selected from TABLE 1. An amino acid sequence of the effector protein is similar to the reference amino acid sequence, when a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value ≥1 is replaced with +1 and any value ≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold ≥1.

In some cases, the effector proteins comprise a RuvC domain. In some embodiments, the RuvC domain may be defined by a single, contiguous sequence, or a set of RuvC subdomains that are not contiguous with respect to the primary amino acid sequence of the protein. An effector protein of the present disclosure may include multiple RuvC subdomains, which may combine to generate a RuvC domain with substrate binding or catalytic activity. For example, an effector protein may include three RuvC subdomains (RuvC-I, RuvC-II, and RuvC-III) that are not contiguous with respect to the primary amino acid sequence of the effector protein but form a RuvC domain once the protein is produced and folds. In many cases, effector proteins comprise a recognition domain with a binding affinity for a guide nucleic acid or for a guide nucleic acid-target nucleic acid heteroduplex. An effector protein may comprise a zinc finger domain.

An effector protein may be small, which may be beneficial for nucleic acid detection or editing (for example, the effector protein may be less likely to adsorb to a surface or another biological species due to its small size). The smaller nature of these effector proteins may allow for them to be more easily packaged and delivered with higher efficiency in the context of genome editing and more readily incorporated as a reagent in an assay. In some embodiments, the length of the effector protein is less than 400 linked amino acid residues. In some embodiments, the length of the effector protein is less than 1200 linked amino acid residues. In some embodiments, the length of the effector protein is less than 900 linked amino acid residues. In some embodiments, the length of the effector protein is less than 800 linked amino acid residues. In some embodiments, the length of the effector protein is less than 500 linked amino acid residues. In some embodiments, the length of the effector protein is about 400 to about 1200 linked amino acids. In some embodiments, the length of the effector protein is about 400 to about 800 linked amino acid residues. In some embodiments, the length of the effector protein is about 650 to about 750 linked amino acids.

### Protospacer Adjacent Motif (PAM) Sequences

Effector proteins of the present disclosure, dimers thereof, and multimeric complexes thereof may cleave or nick a target nucleic acid within or near a protospacer adjacent motif (PAM) sequence of the target nucleic acid. In some embodiments, cleavage occurs within 10, 20, 30, 40 or 50 nucleotides of a 5′ or 3′ terminus of a PAM sequence. In some embodiments, cleavage occurs within 1, 2, 3, 4, 5, 6, 7, 8, 9 or 10 nucleotides of a 5′ or 3′ terminus of a PAM sequence. A target nucleic acid may comprise a PAM sequence adjacent to a target sequence. In some embodiments, systems, compositions and methods comprise a guide nucleic acid or use thereof, wherein the guide nucleic acid comprises a spacer sequence that is complementary to a target sequence that is adjacent to a PAM sequence.

In some embodiments, the PAM is 5′-TTN-3.′ In some embodiments, the effector protein recognizes a PAM sequence as shown in TABLE 8. In some embodiments, the effector protein recognizes a PAM sequence comprising any of the following nucleotide sequences as set forth in TABLE 8. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLE 1 and TABLE 4.

In some embodiments, the PAM is 5′-NNTN-3.′ In some embodiments, the PAM is 5′-TNTR-3.′ In some embodiments, the PAM is 5′-TNTG-3.′ In some embodiments, the effector protein recognizes a PAM sequence as shown in TABLE 9. In some embodiments, the effector protein recognizes a PAM sequence comprising any of the following nucleotide sequences as set forth in TABLE 9. In some embodiments, the effector protein comprises an amino acid sequence that is at least 65%, at least 70%, at least 75%, at least 80%, at least 85%, at least 90%, at least 95%, at least 97%, or at least 98%, at least 99%, or 100% identical to a sequence selected from TABLE 1 and TABLE 6.

### Engineered Proteins

In some embodiments, effector proteins disclosed herein are engineered proteins. Engineered proteins are not identical to a naturally-occurring protein. Engineered proteins may provide enhanced nuclease or nickase activity as compared to a naturally occurring nuclease or nickase. SEQ ID NO: 24 is a non-limiting example of an engineered protein, wherein residue 26 has been modified to an arginine from a leucine at residue 26 of SEQ ID NO: 1. SEQ ID NO: 44 is a non-limiting example of an engineered protein, wherein residue 220 has been modified to an arginine from a leucine at residue 220 of SEQ ID NO: 3.

An engineered protein may comprise a modified form of a wild-type counterpart protein (e.g., an effector protein). The modified form of the wild-type counterpart may comprise an amino acid change (e.g., deletion, insertion, or substitution) that reduces the nucleic acid-cleaving activity of the effector protein relative to the wild-type counterpart. For example, a nuclease domain (e.g., RuvC domain) of an effector protein may be deleted or mutated relative to a wild-type counterpart effector protein so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1, wherein the amino acid substitution is at a position selected from I2, T5, K15, R18, H20, S21, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, S186, K189, S196, S198, K200, 1203, 5205, K206, Y207, H208, N209, Y220, S223, E258, K281, K348, N355, S362, N406, K435, I471, I489, Y490, F491, D495, K496, K498, K500, D501, V502, K504, S505, D506, V521, N568, 5579, Q612, 5638, F701, P707, and a combination thereof. In some embodiments, the polypeptide comprises an amino acid sequence that is 100% identical to SEQ ID NO: 1, with the exception of at least one amino acid substitution relative to SEQ ID NO: 1, wherein the amino acid substitution is at a position selected from I2, T5, K15, R18, H20, 521, L26, N30, E33, E34, A35, K37, K38, R41, N43, Q54, Q79R, K92E, K99R, S108, E109, H110, G111, D113, T114, P116, K118, E119, A121, N132, K135, Q138, V139, N148, L149, E157, E164, E166, E170, Y180, L182, Q183, K184, 5186, K189, 5196, 5198, K200, 1203, S205, K206, Y207, H208, N209, Y220, S223, E258, K281, K348, N355, S362, N406, K435, I471, I489, Y490, F491, D495, K496, K498, K500, D501, V502, K504, S505, D506, V521, N568, S579, Q612, S638, F701, P707, and a combination thereof. In some embodiments, the amino acid substitution is selected from I2X, T5X, K15X, R18X, H20X, S21X, L26X, N30X, E33X, E34X, A35X, K37X, K38X, R41X, N43X, Q54X, Q79RX, K92EX, K99RX, S108X, E109X, H110X, G111X, D113X, T114X, P116X, K118X, E119X, A121X, N132X, K135X, Q138X, V139X, N148X, L149X, E157X, E164X, E166X, E170X, Y180X, L182X, Q183X, K184X, S186X, K189X, S196X, S198X, K200X, I203X, S205X, K206X, Y207X, H208X, N209X, Y220X, S223X, E258X, K281X, K348X, N355X, S362X, N406X, K435X, I471X, I489X, Y490X, F491X, D495X, K496X, K498X, K500X, D501X, V502X, K504X, S505X, D506X, V521X, N568X, S579X, Q612X, S638X, F701X, P707X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1 wherein the amino acid substitution is selected from T5R, L26R, L26K, A121Q, V139R, S198R, S223P, E258K, I471T, S579R, F701R, P707R, K189P, S638K, Q54R, Q79R, Y220S, N406K, E119S, K92E, K435Q, N568D, and V521T, and a combination thereof. In some embodiments, the polypeptide comprises an amino acid sequence that is 100% identical to SEQ ID NO: 65, with the exception of at least one amino acid substitution relative to SEQ ID NO: 1, wherein the amino acid substitution is selected from T5R, L26R, L26K, A121Q, V139R, S198R, S223P, E258K, 1471T, S579R, F701R, P707R, K189P, S638K, Q54R, Q79R, Y220S, N406K, E119S, K92E, K435Q, N568D, and V521T, and a combination thereof. In some aspects, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1 wherein the amino acid substitution is selected from L26K/A121Q, L26R/A121Q, K99R/L149R, K99R/N148R, L149R/H208R, S362R/L26R L26R/N148R, L26R/H208R, N30R/N148R, L26R/K99R, L26R/P707R, L26R/L149R, L26R/N30R, L26R/N355R, L26R/K281R, L26R/S108R, L26R/K348R, T5R/V139R, I2R/V139R, K99R/S186R, L26R/A673G, L26R/Q674R, S579R/L26K, F701R/E258K, T5R/L26K, L26R/K435Q, L26K/E567Q, L26R/G685R, L26R/Q674K, L26R/P699R, L26R/T70E, L26R/Q232R, L26R/T252R, L26R/P679R, L26R/E83K, L26R/E73P, L26R/K248E, L26R, T5R/S223P, S579R/S223P, L26R/S223P, T5R/A121Q, L26R/A696R, S198R/I471T, L26R/N153R, L26R/E682R, L26R/D703R, Q612R/L26K, L26R/I471T, K348R/L26K, S579R/I471T, L26R/V228R, T5R/S638K, S579R/K189P, S579R/E258K, L26R/K260R, L26R/S638K, S579R/Y220S, T5R/I471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q, K348R/Y220S, S198R/K189P, L26R/E242R, L26R/K678R, T5R/N406K, L26R/I158K, T5R/V521T, L26R/N259R, L26R/K257R, L26R/K256R, T5R/K189P, L26R/C405R, S579R/V521T, S579R/N406K, T5R/K92E, T5R/E258K, L26R/I97R, S579R/S638K, T5R/K435Q, F701R/S638K, L26R/L236R, F701R/I471T, Q612R/S223P, F701R/S223P, S198R/E119S, S579R/K92E, L26R/E715R, Q612R/I471T, F701R/Y220S, S198R/S223P, and L26R/K266R, and a combination thereof. In some embodiments, the polypeptide comprises an amino acid sequence that is 100% identical to SEQ ID NO: 1, with the exception of at least one amino acid substitution relative to SEQ ID NO: 1, wherein the amino acid substitution is selected from L26K/A121Q, L26R/A121Q, K99R/L149R, K99R/N148R, L149R/H208R, S362R/L26R L26R/N148R, L26R/H208R, N30R/N148R, L26R/K99R, L26R/P707R, L26R/L149R, L26R/N30R, L26R/N355R, L26R/K281R, L26R/S108R, L26R/K348R, T5R/V139R, I2R/V139R, K99R/S186R, L26R/A673G, L26K/E567Q, L26R/Q674R, S579R/L26K, F701R/E258K, T5R/L26K, L26R/K435Q, L26R/G685R, L26R/Q674K, L26R/P699R, L26R/T70E, L26R/Q232R, L26R/T252R, L26R/P679R, L26R/E83K, L26R/E73P, L26R/K248E, L26R, T5R/S223P, S579R/S223P, L26R/S223P, T5R/A121Q, L26R/A696R, S198R/I471T, L26R/N153R, L26R/E682R, L26R/D703R, Q612R/L26K, L26R/I471T, K348R/L26K, S579R/I471T, L26R/V228R, T5R/S638K, S579R/K189P, S579R/E258K, L26R/K260R, L26R/S638K, S579R/Y220S, T5R/I471T, L26R/F233R, L26R/V521T, F701R/A121Q, L26R/G361R, S198R/E258K, L26R/S472R, T5R/Y220S, L26R/A150K, L26R/S684R, L26R/E157R, L26R/K248R, F701R/L26K, S198R/N406K, S198R/Y220S, S198R/S638K, S198R/V521T, S579R/A121Q, K348R/Y220S, S198R/K189P, L26R/E242R, L26R/K678R, T5R/N406K, L26R/I158K, T5R/V521T, L26R/N259R, L26R/K257R, L26R/K256R, T5R/K189P, L26R/C405R, S579R/V521T, S579R/N406K, T5R/K92E, T5R/E258K, L26R/I97R, S579R/S638K, T5R/K435Q, F701R/S638K, L26R/L236R, F701R/I471T, Q612R/S223P, F701R/S223P, S198R/E119S, S579R/K92E, L26R/E715R, Q612R/I471T, F701R/Y220S, S198R/S223P, and L26R/K266R, and a combination thereof. In some aspects, these engineered effector proteins demonstrate enhanced nuclease activity relative to the wild-type effector protein.

In some embodiments, the polypeptide comprises an amino acid sequence that is 100% identical to SEQ ID NO: 1, with the exception of at least two amino acid substitutions relative to SEQ ID NO: 1, wherein the amino acid substitutions comprise L26K/E567Q. In some embodiments, the polypeptide comprises or consists of SEQ ID NO: 43.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1 wherein the amino acid substitution is selected from E157A, E164A, E164L, E166A, E166I, E170A, I489A, I489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, the polypeptide comprises an amino acid sequence that is 100% identical to SEQ ID NO: 1, with the exception of at least one amino acid substitution relative to SEQ ID NO: 1, wherein the amino acid substitution is selected from E157A, E164A, E164L, E166A, E166I, E170A, I489A, I489S, Y490S, Y490A, F491A, F491S, F491G, D495G, D495R, D495K, K496A, K496S, K498A, K498S, K500A, K500S, D501R, D501G, D501K, V502A, V502S, K504A, K504S, S505R, D506A, and a combination thereof. In some embodiments, these engineered effector proteins comprise a nickase activity.

In some embodiments, the effector protein is an engineered effector protein and comprises an amino acid sequence that is at least 90%, at least 95%, at least 97%, at least 98%, or at least 99% identical to SEQ ID NO: 1, wherein amino acids S478-S505 have been deleted. In some embodiments, the effector protein is an engineered effector protein that is at least 90%, at least 95%, at least 97%, at least 98%, or at least 99% identical to SEQ ID NO: 1, wherein amino acids S478-S505 have been deleted and replaced with SDLYIERGGDPRDVHQQVETKPKGKRKSEIRILKIR (SEQ ID NO: 22) or SDYIVDHGGDPEKVFFETKSKKDKTKRYKRR (SEQ ID NO: 23). In some embodiments, the effector protein is an engineered effector protein and comprises an amino acid sequence that is at least 90%, at least 95%, at least 97%, at least 98%, at least 99% identical, or is 100% identical to SEQ ID NO: 41. In some embodiments, the effector protein is an engineered effector protein and comprises an amino acid sequence that is at least 90%, at least 95%, at least 97%, at least 98%, at least 99% identical, or is 100% identical to SEQ ID NO: 42.

In some embodiments, the effector protein is an engineered effector protein and comprises an amino acid sequence that is at least 90%, at least 95%, at least 97%, at least 98%, or at least 99% identical to SEQ ID NO: 1, wherein the polypeptide comprises at least one amino acid substitution relative to SEQ ID NO: 1 wherein the amino acid substitution is selected from D369A, D369N, D658A, D658N, E567A, E567Q, and a combination thereof. In some embodiments, the polypeptide comprises an amino acid sequence that is 100% identical to SEQ ID NO: 1, with the exception of at least one amino acid substitution relative to SEQ ID NO: 1, wherein the amino acid substitution is selected from D369A, D369N, D658A, D658N, E567A, E567Q, and a combination thereof. In some aspects, these engineered effector proteins demonstrate reduced or abolished nuclease activity relative to the wild-type effector protein. TABLE 3 provides the exemplary amino acid alterations relative to SEQ ID NO: 1 useful in compositions, systems, and methods described herein.

In some embodiments, the effector protein is an engineered effector protein and comprises an amino acid sequence that is at least 90%, at least 95%, at least 97%, at least 98%, or at least 99% identical to SEQ ID NO: 3, wherein the polypeptide comprises at least one amino acid substitution relative to SEQ ID NO: 3, wherein the amino acid substitution is at a position selected from K58, 180, T84, K105, N193, C202, S209, G210, A218, D220, E225, C246, N286, M295, M298, A306, Y315, Q360, and a combination thereof. In some embodiments, the polypeptide comprises an amino acid sequence that is 100% identical to SEQ ID NO: 3, with the exception of at least one amino acid substitution relative to SEQ ID NO: 3, wherein the amino acid substitution is a position selected from K58, 180, T84, K105, N193, C202, S209, G210, A218, D220, E225, C246, N286, M295, M298, A306, Y315, Q360, and a combination thereof. In some embodiments, the amino acid substitution is selected from K58X, I80X, T84X, K105X, N193X, C202X, S209X, G210X, A218X, D220X, E225X, C246X, N286X, M295X, M298X, A306X, Y315X, and Q360X, wherein X is selected from R, K, and H.

In some embodiments, the effector protein is an engineered effector protein and comprises an amino acid sequence that is at least 90%, at least 95%, at least 97%, at least 98%, or at least 99% identical to SEQ ID NO: 3, wherein the polypeptide comprises at least one amino acid substitution relative to SEQ ID NO: 3, wherein the amino acid substitution is selected from I80R, T84R, K105R, C202R, G210R, A218R, D220R, E225R, C246R, Q360R, I80K, T84K, G210K, N193K, C202K, A218K, D220K, E225K, C246K, N286K, A306K, Q360K, I80H, T84H, K105H, G210H, C202H, A218H, D220H, E225H, C246H, Q360H, K58W, S209F, M295W, M298L, Y315M, D220R/A306K and D220R/K250N and a combination thereof. In some embodiments, the polypeptide comprises an amino acid sequence that is 100% identical to SEQ ID NO: 3, with the exception of at least one amino acid substitution relative to SEQ ID NO: 3, wherein the amino acid substitution is selected from I80R, T84R, K105R, C202R, G210R, A218R, D220R, E225R, C246R, Q360R, I80K, T84K, G210K, N193K, C202K, A218K, D220K, E225K, C246K, N286K, A306K, Q360K, I80H, T84H, K105H, G210H, C202H, A218H, D220H, E225H, C246H, Q360H, K58W, S209F, M295W, M298L, Y315M, D220R/A306K, D220R/K250N, D220R/E335Q and a combination thereof. In some aspects, these engineered effector proteins demonstrate enhanced nuclease activity relative to the wild-type effector protein.

In some embodiments, the effector protein is an engineered effector protein and comprises an amino acid sequence that is at least 90%, at least 95%, at least 97%, at least 98%, or at least 99% identical to SEQ ID NO: 3, wherein the polypeptide comprises at least one amino acid substitution relative to SEQ ID NO: 3, wherein the amino acid substitution is selected from D237A, D418A, D418N, E335A, and E335Q, and a combination thereof. In some embodiments, the polypeptide comprises an amino acid sequence that is 100% identical to SEQ ID NO: 3, with the exception of at least one amino acid substitution relative to SEQ ID NO: 3, wherein the amino acid substitution is selected from D237A, D418A, D418N, E335A, and E335Q, and a combination thereof. In some aspects, these engineered effector proteins demonstrate reduced or abolished nuclease activity relative to the wild-type effector protein. TABLE 5 provides the exemplary amino acid alterations relative to SEQ ID NO: 3 useful in compositions, systems, and methods described herein.

In some embodiments, the effector protein is an engineered effector protein and comprises an amino acid sequence that is 100% identical to SEQ ID NO: 3, with the exception of two amino acid substitutions at D220 and E335 relative to SEQ ID NO: 3. In some embodiments, the amino acid substitutions are D220R and 335Q. In some embodiments, the engineered effector protein comprises or consists of SEQ ID NO: 61.

TABLE 4 and TABLE 6 provide exemplary effector protein engineered variant amino acid sequences.

In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 4, wherein the amino acid residue at position 26 is arginine (R) relative to SEQ ID NO: 1. In some embodiments, an effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 6, wherein the amino acid residue at position 220 is arginine (R) relative to SEQ ID NO: 3. Bold and underlined text in TABLE 4 represents an amino acid mutation at the position, wherein the mutation is relative to SEQ ID NO: 1. Bold and underlined text in TABLE 6 represents an amino acid mutation at the position, wherein the mutation is relative to SEQ ID NO: 3.

In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 4, wherein the amino acid residue at position 26, relative to SEQ ID NO: 24, remains unchanged. In other words, the residue of the amino acid sequence that aligns with position 26 of SEQ ID NO: 24 is an arginine.

In certain embodiments, compositions comprise an effector protein and a guide nucleic acid, wherein the effector protein comprises an amino acid sequence that is at least 65%, at least 70%, at least 75%, at least 80%, at least 85%, at least 90%, at least 95%, at least 97%, or at least 98%, at least 99%, or 100% identical to any one of the sequences as set forth in TABLE 6, wherein the amino acid residue at position 220, relative to SEQ ID NO: 44, remains unchanged. In other words, the residue of the amino acid sequence that aligns with position 220 of SEQ ID NO: 44 is an arginine.

In certain embodiments, the amino acid sequence of the effector protein is based on SEQ ID NO: 1 and is modified at position 26. In some embodiments the modification at position 26 is from leucine to arginine (L26R). In some embodiments, the amino acid sequence of the effector protein is at least 95%, 96%, 97%, 98%, or 99% identical to SEQ ID NO: 24. In some embodiments, the amino acid sequence of the effector protein comprises or consists of SEQ ID NO: 24.

In certain embodiments, the amino acid sequence of the effector protein is based on SEQ ID NO: 1 and is modified at position 109. In some embodiments the modification at position 109 is from glutamic acid to arginine (E109R). In some embodiments, the amino acid sequence of the effector protein is at least 95%, 96%, 97%, 98%, or 99% identical to SEQ ID NO: 28. In some embodiments, the amino acid sequence of the effector protein comprises or consists of SEQ ID NO: 28.

In certain embodiments, the amino acid sequence of the effector protein is based on SEQ ID NO: 1 and is modified at position 208. In some embodiments the modification at position 208 is from histidine to arginine (H208R). In some embodiments, the amino acid sequence of the effector protein is at least 95%, 96%, 97%, 98%, or 99% identical to SEQ ID NO: 29. In some embodiments, the amino acid sequence of the effector protein comprises or consists of SEQ ID NO: 29.

In certain embodiments, the amino acid sequence of the effector protein is based on SEQ ID NO: 1 and is modified at position 184. In some embodiments the modification at position 184 is from lysine to arginine (K184R). In some embodiments, the amino acid sequence of the effector protein is at least 95%, 96%, 97%, 98%, or 99% identical to SEQ ID NO: 30. In some embodiments, the amino acid sequence of the effector protein comprises or consists of SEQ ID NO: 30.

In certain embodiments, the amino acid sequence of the effector protein is based on SEQ ID NO: 1 and is modified at position 38. In some embodiments the modification at position 38 is from lysine to arginine (K38R). In some embodiments, the amino acid sequence of the effector protein is at least 95%, 96%, 97%, 98%, or 99% identical to SEQ ID NO: 31. In some embodiments, the amino acid sequence of the effector protein comprises or consists of SEQ ID NO: 31.

In certain embodiments, the amino acid sequence of the effector protein is based on SEQ ID NO: 1 and is modified at position 182. In some embodiments the modification at position 182 is from leucine to arginine (L182R). In some embodiments, the amino acid sequence of the effector protein is at least 95%, 96%, 97%, 98%, or 99% identical to SEQ ID NO: 32. In some embodiments, the amino acid sequence of the effector protein comprises or consists of SEQ ID NO: 32.

In certain embodiments, the amino acid sequence of the effector protein is based on SEQ ID NO: 1 and is modified at position 183. In some embodiments the modification at position 183 is from glutamine to arginine (Q183R). In some embodiments, the amino acid sequence of the effector protein is at least 95%, 96%, 97%, 98%, or 99% identical to SEQ ID NO: 33. In some embodiments, the amino acid sequence of the effector protein comprises or consists of SEQ ID NO: 33.

In certain embodiments, the amino acid sequence of the effector protein is based on SEQ ID NO: 1 and is modified at position 108. In some embodiments the modification at position 108 is from serine to arginine (S108R). In some embodiments, the amino acid sequence of the effector protein is at least 95%, 96%, 97%, 98%, or 99% identical to SEQ ID NO: 34. In some embodiments, the amino acid sequence of the effector protein comprises or consists of SEQ ID NO: 34.

In certain embodiments, the amino acid sequence of the effector protein is based on SEQ ID NO: 1 and is modified at position 198. In some embodiments the modification at position 198 is from serine to arginine (S198R). In some embodiments, the amino acid sequence of the effector protein is at least 95%, 96%, 97%, 98%, or 99% identical to SEQ ID NO: 35. In some embodiments, the amino acid sequence of the effector protein comprises or consists of SEQ ID NO: 35.

In certain embodiments, the amino acid sequence of the effector protein is based on SEQ ID NO: 1 and is modified at position 114. In some embodiments the modification at position 114 is from threonine to arginine (T114R). In some embodiments, the amino acid sequence of the effector protein is at least 95%, 96%, 97%, 98%, or 99% identical to SEQ ID NO: 36. In some embodiments, the amino acid sequence of the effector protein comprises or consists of SEQ ID NO: 36.

In certain embodiments, the amino acid sequence of the effector protein is based on SEQ ID NO: 3 and is modified at position 286. In some embodiments the modification at position 286 is from asparagine to lysine (N286K). In some embodiments, the amino acid sequence of the effector protein is at least 95%, 96%, 97%, 98%, or 99% identical to SEQ ID NO: 54. In some embodiments, the amino acid sequence of the effector protein comprises or consists of SEQ ID NO: 54.

In certain embodiments, the amino acid sequence of the effector protein is based on SEQ ID NO: 3 and is modified at position 225. In some embodiments the modification at position 225 is from glutamic acid to lysine (E225K). In some embodiments, the amino acid sequence of the effector protein is at least 95%, 96%, 97%, 98%, or 99% identical to SEQ ID NO: 53. In some embodiments, the amino acid sequence of the effector protein comprises or consists of SEQ ID NO: 53.

In certain embodiments, the amino acid sequence of the effector protein is based on SEQ ID NO: 3 and is modified at position 80. In some embodiments the modification at position 80 is from isoleucine to lysine (I80K). In some embodiments, the amino acid sequence of the effector protein is at least 95%, 96%, 97%, 98%, or 99% identical to SEQ ID NO: 52. In some embodiments, the amino acid sequence of the effector protein comprises or consists of SEQ ID NO: 52.

In certain embodiments, the amino acid sequence of the effector protein is based on SEQ ID NO: 3 and is modified at position 209. In some embodiments the modification at position 209 is from serine to phenylalanine (S209F). In some embodiments, the amino acid sequence of the effector protein is at least 95%, 96%, 97%, 98%, or 99% identical to SEQ ID NO: 51. In some embodiments, the amino acid sequence of the effector protein comprises or consists of SEQ ID NO: 51.

In certain embodiments, the amino acid sequence of the effector protein is based on SEQ ID NO: 3 and is modified at position 315. In some embodiments the modification at position 315 is from tyrosine to methionine (Y315M). In some embodiments, the amino acid sequence of the effector protein is at least 95%, 96%, 97%, 98%, or 99% identical to SEQ ID NO: 50. In some embodiments, the amino acid sequence of the effector protein comprises or consists of SEQ ID NO: 50.

In certain embodiments, the amino acid sequence of the effector protein is based on SEQ ID NO: 3 and is modified at position 193. In some embodiments the modification at position 193 is from asparagine to lysine (N193K). In some embodiments, the amino acid sequence of the effector protein is at least 95%, 96%, 97%, 98%, or 99% identical to SEQ ID NO: 49. In some embodiments, the amino acid sequence of the effector protein comprises or consists of SEQ ID NO: 49.

In certain embodiments, the amino acid sequence of the effector protein is based on SEQ ID NO:3 and is modified at position 298. In some embodiments the modification at position 298 is from methionine to leucine (M298L). In some embodiments, the amino acid sequence of the effector protein is at least 95%, 96%, 97%, 98%, or 99% identical to SEQ ID NO: 48. In some embodiments, the amino acid sequence of the effector protein comprises or consists of SEQ ID NO: 48.

In certain embodiments, the amino acid sequence of the effector protein is based on SEQ ID NO: 3 and is modified at position 295. In some embodiments the modification at position 295 is from methionine to tryptophan (M295W). In some embodiments, the amino acid sequence of the effector protein is at least 95%, 96%, 97%, 98%, or 99% identical to SEQ ID NO: 47. In some embodiments, the amino acid sequence of the effector protein comprises or consists of SEQ ID NO: 47.

In certain embodiments, the amino acid sequence of the effector protein is based on SEQ ID NO: 3 and is modified at position 306. In some embodiments the modification at position 306 is from alanine to lysine (A306K). In some embodiments, the amino acid sequence of the effector protein is at least 95%, 96%, 97%, 98%, or 99% identical to SEQ ID NO: 55. In some embodiments, the amino acid sequence of the effector protein comprises or consists of SEQ ID NO: 55.

In certain embodiments, the amino acid sequence of the effector protein is based on SEQ ID NO: 3 and is modified at position 218. In some embodiments the modification at position 218 is from alanine to lysine (A218K). In some embodiments, the amino acid sequence of the effector protein is at least 95%, 96%, 97%, 98%, or 99% identical to SEQ ID NO: 46. In some embodiments, the amino acid sequence of the effector protein comprises or consists of SEQ ID NO: 46.

In certain embodiments, the amino acid sequence of the effector protein is based on SEQ ID NO: 3 and is modified at position 58. In some embodiments the modification at position 58 is from lysine to tryptophan (K58W). In some embodiments, the amino acid sequence of the effector protein is at least 95%, 96%, 97%, 98%, or 99% identical to SEQ ID NO: 45. In some embodiments, the amino acid sequence of the effector protein comprises or consists of SEQ ID NO: 45.

In some embodiments any of the following modifications in SEQ ID NOs: 24 and 28-36 enhances nuclease activity: L26R, E109R, H208R, K184R, K38R, L182R, Q183R, S108R, S198R, and T114R.

In some embodiments any of the following modifications in SEQ ID NOs: 44 and 45-55 enhances nuclease activity: D220R, N286K, E225K, I80K, S209F, Y315M, N193K, M298L, M295W, A306K, A218K, or K58W.

### Nuclease-Dead Effector Proteins

In some embodiments, the effector protein may comprise an enzymatically inactive and/or “dead” (abbreviated by “d”) effector protein in combination (e.g., fusion) with a polypeptide comprising recombinase activity. In some embodiments, nuclease-dead effector protein may also be referred to as a catalytically inactive effector protein. Although an effector protein normally has nuclease activity, in some embodiments, an effector protein does not have nuclease activity. In some embodiments, an effector protein comprising a nuclease-dead effector protein, wherein the nuclease-dead effector protein comprising an amino acid sequence that is at least 60%, at least 65%, at least 70%, at least 75%, at least 80%, at least 85%, at least 90%, at least 92%, at least 95%, at least 97%, at least 98%, at least 99%, or 100% identical to any one of the sequences recited in TABLE 1, TABLE 4 or TABLE 6. In some embodiments, the effector protein comprising an amino acid sequence that is at least 60%, at least 65%, at least 70%, at least 75%, at least 80%, at least 85%, at least 90%, at least 92%, at least 95%, at least 97%, at least 98%, at least 99%, or 100% identical to any one of the sequences recited in TABLE 1, TABLE 4 or TABLE 6, wherein the effector protein is modified or engineered to be a nuclease-dead effector protein.

Catalytically inactive effector proteins may comprise a modified form of a wildtype counterpart. The modified form of the wildtype counterpart may comprise an amino acid change (e.g., deletion, insertion, or substitution) that reduces the nucleic acid-cleaving activity of the effector protein. In such embodiments, the catalytically inactive effector protein may also be referred to as a catalytically reduced effector protein. For example, a nuclease domain (e.g., HEPN domain, RuvC domain) of an effector protein can be deleted or mutated so that it is no longer functional or comprises reduced nuclease activity. The modified form of the effector protein may have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of an effector protein may have no substantial nucleic acid-cleaving activity. When an effector protein is a modified form that has no substantial nucleic acid-cleaving activity, it may be referred to as enzymatically inactive and/or dead. A dead effector polypeptide (e.g., catalytically inactive effector protein) may bind to a target nucleic acid but may not cleave the target nucleic acid. A dead effector polypeptide (e.g., catalytically inactive effector protein) may associate with a guide nucleic acid to activate or repress transcription of a target nucleic acid.

In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, or at least 99% identical to SEQ ID NO: 1, and wherein the effector protein further comprises one or more alterations selected from D369A, D369N, E567A, E567Q, D658A and D658N.

In certain embodiments, the amino acid sequence of the dCas protein is based on SEQ ID NO:1 and is modified at position 369. In some embodiments the modification at position 369 is from aspartic acid to alanine (D369A). In some embodiments, the amino acid sequence of the dCas protein is at least 95%, 96%, 97%, 98%, or 99% identical to SEQ ID NO: 37. In some embodiments, the amino acid sequence of the dCas protein comprises or consists of SEQ ID NO: 37.

In certain embodiments, the amino acid sequence of the dCas protein is based on SEQ ID NO: 1 and is modified at position 369. In some embodiments the modification at position 369 is from aspartic acid to asparagine (D369N). In some embodiments, the amino acid sequence of the dCas protein is at least 95%, 96%, 97%, 98%, or 99% identical to SEQ ID NO: 38. In some embodiments, the amino acid sequence of the dCas protein comprises or consists of SEQ ID NO: 38.

In certain embodiments, the amino acid sequence of the dCas protein is based on SEQ ID NO: 1 and is modified at position 658. In some embodiments the modification at position 658 is from aspartic acid to alanine (D658A). In some embodiments, the amino acid sequence of the dCas protein is at least 95%, 96%, 97%, 98%, or 99% identical to SEQ ID NO: 39. In some embodiments, the amino acid sequence of the dCas protein comprises or consists of SEQ ID NO: 39.

In certain embodiments, the amino acid sequence of the dCas protein is based on SEQ ID NO: 1 and is modified at position 658. In some embodiments the modification at position 658 is from aspartic acid to asparagine (D658N). In some embodiments, the amino acid sequence of the dCas protein is at least 95%, 96%, 97%, 98%, or 99% identical to SEQ ID NO: 40. In some embodiments, the amino acid sequence of the dCas protein comprises or consists of SEQ ID NO: 40.

In certain embodiments, the amino acid sequence of the dCas protein is based on SEQ ID NO: 1 and is modified at position 567. In some embodiments the modification at position 567 is from glutamine acid to alanine (E567A). In some embodiments, the amino acid sequence of the dCas protein is at least 95%, 96%, 97%, 98%, or 99% identical to SEQ ID NO: 26. In some embodiments, the amino acid sequence of the dCas protein comprises or consists of SEQ ID NO: 26.

In certain embodiments, the amino acid sequence of the dCas protein is based on SEQ ID NO: 1 and is modified at position 567. In some embodiments the modification at position 567 is from glutamic acid to glutamine (E567Q). In some embodiments, the amino acid sequence of the dCas protein is at least 95%, 96%, 97%, 98%, or 99% identical to SEQ ID NO: 27. In some embodiments, the amino acid sequence of the dCas protein comprises or consists of SEQ ID NO: 27.

In some embodiments, a nuclease-dead effector protein comprises an amino acid sequence that is at least 60%, at least 65%, at least 70%, at least 75%, at least 80%, at least 85%, at least 90%, at least 92%, at least 95%, at least 97%, at least 98%, or at least 99% identical to SEQ ID NO: 3, and wherein the effector protein further comprises one or more alterations selected from D237A, D418A, D418N, E335A, and E335Q.

In certain embodiments, the amino acid sequence of the dCas protein is based on SEQ ID NO: 3 and is modified at position 335. In some embodiments the modification at position 335 is from glutamic acid to glutamine (E335Q). In some embodiments, the amino acid sequence of the dCas protein is at least 95%, 96%, 97%, 98%, or 99% identical to SEQ ID NO: 56. In some embodiments, the amino acid sequence of the dCas protein comprises or consists of SEQ ID NO: 56.

In certain embodiments, the amino acid sequence of the dCas protein is based on SEQ ID NO: 3 and is modified at position 237. In some embodiments the modification at position 237 is from aspartic acid to alanine (D237A). In some embodiments, the amino acid sequence of the dCas protein is at least 95%, 96%, 97%, 98%, or 99% identical to SEQ ID NO: 57. In some embodiments, the amino acid sequence of the dCas protein comprises or consists of SEQ ID NO: 57.

In certain embodiments, the amino acid sequence of the dCas protein is based on SEQ ID NO: 3 and is modified at position 418. In some embodiments the modification at position 418 is from aspartic acid to alanine (D418A). In some embodiments, the amino acid sequence of the dCas protein is at least 95%, 96%, 97%, 98%, or 99% identical to SEQ ID NO: 58. In some embodiments, the amino acid sequence of the dCas protein comprises or consists of SEQ ID NO: 58.

In certain embodiments, the amino acid sequence of the dCas protein is based on SEQ ID NO: 3 and is modified at position 418. In some embodiments the modification at position 418 is from aspartic acid to asparagine (D418N). In some embodiments, the amino acid sequence of the dCas protein is at least 95%, 96%, 97%, 98%, or 99% identical to SEQ ID NO: 59. In some embodiments, the amino acid sequence of the dCas protein comprises or consists of SEQ ID NO: 59.

In certain embodiments, the amino acid sequence of the dCas protein is based on SEQ ID NO: 3 and is modified at position 335. In some embodiments the modification at position 335 is from glutamic acid to alanine (E335A). In some embodiments, the amino acid sequence of the dCas protein is at least 95%, 96%, 97%, 98%, or 99% identical to SEQ ID NO: 60. In some embodiments, the amino acid sequence of the dCas protein comprises or consists of SEQ ID NO: 60.

### Fusion Proteins

In some embodiments, compositions, systems, and methods comprise a fusion protein or uses thereof. A fusion protein generally comprises an effector protein and a fusion partner. In some embodiments, the fusion partner comprises a polypeptide or peptide that is linked to the effector protein. In some embodiments, the fusion partner is not linked to the effector protein but is brought into proximity of the effector protein by other means. By way of non-limiting example, a fusion partner protein may comprise a peptide that binds an aptamer of a guide nucleic acid, wherein the effector protein is also capable of binding the guide nucleic acid, the guide nucleic acid thereby bringing the fusion partner into proximity of the effector protein. In some embodiments, the fusion partner is capable of binding or being bound by an effector protein. In some embodiments, the fusion partner and the effector protein are both capable of binding or being bound by an additional protein or moiety, the additional protein or moiety thereby bringing the fusion partner into proximity of the effector protein. In some embodiments, the fusion protein is a heterologous peptide or polypeptide as described herein. In some embodiments, the amino terminus of the fusion partner is linked to the carboxy terminus of the effector protein. In some embodiments, the carboxy terminus of the fusion partner protein is linked to the amino terminus of the effector protein by the linker. In some embodiments, the fusion partner is not an effector protein as described herein. In some embodiments, the fusion partner comprises a second effector protein or a multimeric form thereof. Accordingly, in some embodiments, the fusion protein comprises more than one effector protein. In such embodiments, the fusion protein can comprise at least two effector proteins that are same. In some embodiments, the fusion protein comprises at least two effector proteins that are different. In some embodiments, the multimeric form is a homomeric form. In some embodiments, the multimeric form is a heteromeric form. Unless otherwise indicated, reference to effector proteins throughout the present disclosure include fusion proteins comprising the effector protein described herein and a fusion partner.

In some embodiments, a fusion partner imparts some function or activity to a fusion protein that is not provided by an effector protein. Such activities may include but are not limited to nuclease activity, methyltransferase activity, demethylase activity, DNA repair activity, DNA damage activity, deamination activity, dismutase activity, alkylation activity, depurination activity, oxidation activity, dimer forming activity (e.g., pyrimidine dimer forming activity), integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity, modification of a polypeptide associated with target nucleic acid (e.g., a histone), and/or signaling activity.

In some embodiments, a fusion partner may provide signaling activity. In some embodiments, a fusion partner may inhibit or promote the formation of multimeric complex of an effector protein. In an additional example, the fusion partner may directly or indirectly edit a target nucleic acid. Edits can be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid.

In some embodiments of the above, the effector protein amino acid sequence comprises a nuclear localization signal.

In some embodiments of the above, the composition further comprises an additional guide RNA that binds a different portion of the target nucleic acid than the guide RNA.

In some embodiments of the above, the guide RNA comprises at least one sequence that is at least 80%, at least 85%, at least 90%, at least 95% or 100% identical to a sequence selected from any one of TABLES 10-15 and SEQ ID NOs: 90-92.

### Nucleic Acid Modification Activity

In some embodiments, fusion partners have enzymatic activity that modifies a nucleic acid, such as a target nucleic acid. In some embodiments, the target nucleic acid may comprise or consist of a ssRNA, dsRNA, ssDNA, or a dsDNA. Examples of enzymatic activity that modifies the target nucleic acid include, but are not limited to: nuclease activity, which comprises the enzymatic activity of an enzyme which allows the enzyme to cleave the phosphodiester bonds between the nucleotide subunits of nucleic acids, such as that provided by a restriction enzyme, or a nuclease (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y, human immunodeficiency virus type 1 integrase (IN), Tn3 resolvase); transposase activity; recombinase activity such as that provided by a recombinase (e.g., catalytic domain of Gin recombinase); polymerase activity; ligase activity; helicase activity; photolyase activity; and glycosylase activity.

In some embodiments, fusion partners target a ssRNA, dsRNA, ssDNA, or a dsDNA. In some embodiments, fusion partners target ssRNA. Non-limiting examples of fusion partners for targeting ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

It is understood that a fusion partner may include an entire protein, or in some embodiments, may include a fragment of the protein (e.g., a functional domain). In some embodiments, the functional domain binds or interacts with a nucleic acid, such as ssRNA, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid editing, nucleic acid mutating, nucleic acid modifying, nucleic acid cleaving, protein binding or combinations thereof. The absence of the functional domain, including mutations of the functional domain, would abolish or reduce activity.

Accordingly, fusion partners may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CID1 and terminal uridylate transferase); and other suitable domains that affect nucleic acid modifications.

In some embodiments, an effector protein is a fusion protein, wherein the effector protein is linked to a chromatin-modifying enzyme. In some embodiments, the fusion protein chemically modifies a target nucleic acid, for example by methylating, demethylating, or acetylating the target nucleic acid in a sequence specific or non-specific manner.

### Base Editors

In some embodiments, fusion partners edit a nucleobase of a target nucleic acid. Fusion proteins comprising such a fusion partner and an effector protein may be referred to as base editors. Such a fusion partner may be referred to as a base editing enzyme. In some embodiments, a base editor comprises a base editing enzyme variant that differs from a naturally occurring base editing enzyme, but it is understood that any reference to a base editing enzyme herein also refers to a base editing enzyme variant. In some embodiments, a base editor may be a fusion protein comprising a base editing enzyme linked to an effector protein. In some embodiments, the amino terminus of the fusion partner protein is linked to the carboxy terminus of the effector protein by the linker. In some embodiments, the carboxy terminus of the fusion partner protein is linked to the amino terminus of the effector protein by the linker. The base editor may be functional when the effector protein is coupled to a guide nucleic acid. The base editor may be functional when the effector protein is coupled to a guide nucleic acid. The guide nucleic acid imparts sequence specific activity to the base editor. By way of non-limiting example, the effector protein may comprise a catalytically inactive effector protein (e.g., a catalytically inactive variant of an effector protein described herein). Also, by way of non-limiting example, the base editing enzyme may comprise deaminase activity. Additional base editors are described herein.

In some embodiments, base editors are capable of catalyzing editing (e.g., a chemical modification) of a nucleobase of a nucleic acid molecule, such as DNA or RNA (single stranded or double stranded). In some embodiments, a base editing enzyme, and therefore a base editor, is capable of converting an existing nucleobase to a different nucleobase, such as: an adenine (A) to guanine (G); cytosine (C) to thymine (T); cytosine (C) to guanine (G); uracil (U) to cytosine (C); guanine (G) to adenine (A); hydrolytic deamination of an adenine or adenosine, or methylation of cytosine (e.g., CpG, CpA, CpT or CpC). In some embodiments, base editors edit a nucleobase on a ssDNA. In some embodiments, base editors edit a nucleobase on both strands of dsDNA. In some embodiments, base editors edit a nucleobase of an RNA.

In some embodiments, a base editing enzyme itself may or may not bind to the nucleic acid molecule containing the nucleobase. In some embodiments, upon binding to its target locus in the target nucleic acid (e.g., a DNA molecule), base pairing between the guide nucleic acid and target strand leads to displacement of a small segment of ssDNA in an “R-loop”. In some embodiments, DNA bases within the R-loop are edited by the base editor having the deaminase enzyme activity. In some embodiments, base editors for improved efficiency in eukaryotic cells comprise a catalytically inactive effector protein that may generate a nick in the non-edited strand, inducing repair of the non-edited strand using the edited strand as a template.

In some embodiments, a base editing enzyme comprises a deaminase enzyme. Exemplary deaminases are described in US20210198330, WO2021041945, WO2021050571A1, and WO2020123887, all of which are incorporated herein by reference in their entirety. Exemplary deaminase domains are described WO2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety. In some embodiments, the deaminase functions as a monomer. In some embodiments, the deaminase functions as heterodimer with an additional protein. In some embodiments, base editors comprise a DNA glycosylase inhibitor (e.g., an uracil glycosylase inhibitor (UGI) or uracil N-glycosylase (UNG)). In some embodiments, the fusion partner is a deaminase, e.g., ADAR1/2, ADAR-2, AID, or any function variant thereof.

In some embodiments, a base editor is a cytosine base editor (CBE). In some embodiments, the CBE may convert a cytosine to a thymine. In some embodiments, a cytosine base editing enzyme may accept ssDNA as a substrate but may not be capable of cleaving dsDNA, as linked to a catalytically inactive effector protein. In some embodiments, when bound to its cognate DNA, the catalytically inactive effector protein of the CBE may perform local denaturation of the DNA duplex to generate an R-loop in which the DNA strand not paired with a guide nucleic acid exists as a disordered single-stranded bubble. In some embodiments, the catalytically inactive effector protein generated ssDNA R-loop may enable the CBE to perform efficient and localized cytosine deamination in vitro. In some embodiments, deamination activity is exhibited in a window of about 4 to about 10 base pairs. In some embodiments, fusion to the catalytically inactive effector protein presents a target site to the cytosine base editing enzyme in high effective molarity, which may enable the CBE to deaminate cytosines located in a variety of different sequence motifs, with differing efficacies. In some embodiments, the CBE is capable of mediating RNA-programmed deamination of target cytosines in vitro or in vivo. In some embodiments, the cytosine base editing enzyme is a cytidine deaminase. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C⋅G-to-G⋅C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U⋅G in DNA is initiated by a UNG, which recognizes a U⋅G mismatch and cleaves the glyosidic bond between a uracil and a deoxyribose backbone of DNA. In some embodiments, BER results in the reversion of the U⋅G intermediate created by the first CBE back to a C⋅G base pair. In some embodiments, the UNG may be inhibited by fusion of a UGI. In some embodiments, the CBE comprises a UGI. In some embodiments, a C-terminus of the CBE comprises the UGI. In some embodiments, the UGI is a small protein from bacteriophage PBS. In some embodiments, the UGI is a DNA mimic that potently inhibits both human and bacterial UNG. In some embodiments, the UGI inhibitor is any protein or polypeptide that inhibits UNG. In some embodiments, the CBE may mediate efficient base editing in bacterial cells and moderately efficient editing in mammalian cells, enabling conversion of a C⋅G base pair to a T⋅A base pair through a U⋅G intermediate. In some embodiments, the CBE is modified to increase base editing efficiency while editing more than one strand of DNA.

In some embodiments, a CBE nicks a non-edited DNA strand. In some embodiments, the non-edited DNA strand nicked by the CBE biases cellular repair of a U⋅G mismatch to favor a U⋅A outcome, elevating base editing efficiency. In some embodiments, a APOBEC1-nickase-UGI fusion efficiently edits in mammalian cells, while minimizing frequency of non-target indels. In some embodiments, base editors do not comprise a functional fragment of the base editing enzyme. In some embodiments, base editors do not comprise a function fragment of a UGI, where such a fragment may be capable of excising a uracil residue from DNA by cleaving an N-glycosidic bond.

In some embodiments, the fusion protein further comprises a non-protein uracil-DNA glycosylase inhibitor (npUGI). In some embodiments, the npUGI is selected from a group of small molecule inhibitors of uracil-DNA glycosylase (UDG), or a nucleic acid inhibitor of UDG. In some embodiments, the npUGI is a small molecule derived from uracil. Examples of small molecule non-protein uracil-DNA glycosylase inhibitors, fusion proteins, and Cas-CRISPR systems comprising base editing activity are described in WO2021087246, which is incorporated by reference in its entirety.

In some embodiments, a cytosine base editing enzyme, and therefore a cytosine base editor, is a cytidine deaminase. In some embodiments, the cytidine deaminase base editor is generated by ancestral sequence reconstruction as described in WO2019226953, which is hereby incorporated by reference in its entirety. Non-limiting exemplary cytidine deaminases suitable for use with effector proteins described herein include: APOBEC1, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, and saBE4-Gam as described in WO2021163587, WO2021087246, WO2021062227, and WO2020123887, which are incorporated herein by reference in their entirety.

In some embodiments, a base editor is a cytosine to guanine base editor (CGBE). A CGBE may convert a cytosine to a guanine.

In some embodiments, a base editor is an adenine base editor (ABE). An ABE may convert an adenine to a guanine. In some embodiments, an ABE converts an A⋅T base pair to a G⋅C base pair. In some embodiments, the ABE converts a target A⋅T base pair to G⋅C in vivo or in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. Non-limiting exemplary adenine base editing enzymes suitable for use with effector proteins described herein include: ABE8e, ABE8.20m, APOBEC3A, Anc APOBEC (a.k.a. AncBE4Max), and BtAPOBEC2. Non-limiting exemplary ABEs suitable for use herein include: ABE7, ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, and ABE8.24d. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871.

In some embodiments, the ABE is ABE8e and comprises an amino acid sequence that is at least at least 90%, at least 95%, at least 97%, at least 98%, or at least 99% identical to SEQ ID NO: 64. In some embodiments, the ABE is ABE8e and comprises or consists of SEQ ID NO: 64.

In some embodiments, the present disclosure provides a fusion protein comprising an effector protein described herein and a base editor described herein. In some embodiments, the fusion protein comprises, from N-terminus to C-terminus, an effector protein and a base editing enzyme. In some embodiments, the fusion protein comprises, from N-terminus to C-terminus, a base editing enzyme and an effector protein. In some embodiments, the base editing enzyme is ABE8e.

In some embodiments, the fusion protein described herein comprises an effector protein comprising an amino acid sequence that is at least 90%, 91%, 92%, 93%, 94%, 95%, 96%, 97%, 98%, or 99% identical to SEQ ID NO: 3 and a base editing enzyme comprising an amino acid sequence that is at least 90%, 91%, 92%, 93%, 94%, 95%, 96%, 97%, 98%, or 99% identical to SEQ ID NO: 64. In some embodiments, the fusion protein described herein comprises an effector protein comprising or consisting of SEQ ID NO: 3 and a base editing enzyme comprising or consisting of SEQ ID NO: 64. In some embodiments, the fusion protein comprises a linker sequence comprising SEQ ID NO: 67. In some embodiments, the fusion protein comprises an amino acid sequence that is at least at least 90%, at least 95%, at least 97%, at least 98%, or at least 99% identical to SEQ ID NO: 65. In some embodiments, the fusion protein comprises or consists of SEQ ID NO: 65.

In some embodiments, the fusion protein described herein comprises an effector protein comprising an amino acid sequence that is at least 90%, 91%, 92%, 93%, 94%, 95%, 96%, 97%, 98%, or 99% identical to SEQ ID NO: 1 and a base editing enzyme comprising an amino acid sequence that is at least 90%, 91%, 92%, 93%, 94%, 95%, 96%, 97%, 98%, or 99% identical to SEQ ID NO: 64. In some embodiments, the fusion protein described herein comprises an effector protein comprising or consisting of SEQ ID NO: 1 and a base editing enzyme comprising or consisting of SEQ ID NO: 64. In some embodiments, the fusion protein comprises a linker sequence comprising SEQ ID NO: 67. In some embodiments, the fusion protein comprises an amino acid sequence that is at least at least 90%, at least 95%, at least 97%, at least 98%, or at least 99% identical to SEQ ID NO: 66. In some embodiments, the fusion protein and comprises or consists of SEQ ID NO: 66. Exemplary fusion proteins are provided in TABLE 7.

In some embodiments, an adenine base editing enzyme of an ABE is an adenosine deaminase. Non-limiting exemplary adenosine base editing enzymes suitable for use herein include ABE9. In some embodiments, the ABE comprises an engineered adenosine deaminase enzyme capable of acting on ssDNA. The engineered adenosine deaminase enzyme may be an adenosine deaminase variant that differs from a naturally occurring deaminase. Relative to the naturally occurring deaminase, the adenosine deaminase variant may comprise one or more amino acid alteration, including a V82S alteration, a T166R alteration, a Y147T alteration, a Y147R alteration, a Q154S alteration, a Y123H alteration, a Q154R alteration, or a combination thereof.

In some embodiments, a base editor comprises a deaminase dimer. In some embodiments, the base editor further comprising a base editing enzyme and an adenine deaminase (e.g., TadA). In some embodiments, the adenosine deaminase is a TadA monomer (e.g., Tad*7.10, TadA*8 or TadA*9). In some embodiments, the adenosine deaminase is a TadA*8 variant (e.g., any one of TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entirety). In some embodiments, the base editor comprises a base editing enzyme linked to TadA by a linker (e.g., wherein the base editing enzyme is linked to TadA at N-terminus or C-terminus by a linker).

In some embodiments, a base editing enzyme is a deaminase dimer comprising an ABE. In some embodiments, the deaminase dimer comprises an adenosine deaminase. In some embodiments, the deaminase dimer comprises TadA linked to a suitable adenine base editing enzyme including an: ABE8e, ABE8.20m, APOBEC3A, Anc APOBEC (a.k.a. AncBE4Max), BtAPOBEC2, and variants thereof. In some embodiments, the adenine base editing enzyme is linked to amino-terminus or the carboxy-terminus of TadA.

In some embodiments, RNA base editors comprise an adenosine deaminase. In some embodiments, ADAR proteins bind to RNAs and alter their sequence by changing an adenosine into an inosine. In some embodiments, RNA base editors comprise an effector protein that is activated by or binds RNA.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions, systems, and methods described herein comprise a base editor and a guide nucleic acid, wherein the guide nucleic acid directs the base editor to a sequence in a target gene.

### Precision Editing Systems

In some embodiments, the fusion partner comprises a polymerase. In some embodiments, the fusion partner is an RNA-directed DNA polymerase (RDDP). In some embodiments, the RDDP is a reverse transcriptase.

In some embodiments, the RDDP that is capable of catalyzing the modification of the target nucleic acid forms a complex with an extended guide RNA. In some embodiments, the extended guide RNA comprises (not necessarily in this order): a first region (also referred to as a repeat sequence) that interacts with an effector protein; a second region comprising a spacer sequence that is complementary to a target sequence of a first strand of a target dsDNA molecule; a third region comprising a template sequence that is complementary to at least a portion of the target sequence on the non-target strand of the target dsDNA molecule with the exception of at least one nucleotide; and a fourth region comprising a primer binding sequence that hybridizes to a primer sequence of the target dsDNA molecule that is formed when target nucleic acid is cleaved. The third region or template sequence may comprise a nucleotide having a different nucleobase than that of a nucleotide at the corresponding position in the target nucleic acid when the template sequence and the target sequence are aligned for maximum identity. In some embodiments, there is a linker between any one of the first, second, third and fourth regions. In some embodiments, the linker comprises a nucleotide. In some embodiments, the linker comprises multiple nucleotides.

In some embodiments, the third and fourth regions are 5′ of the first and second regions. In some embodiments, the order of the regions of the extended guide RNA from 5′ to 3′ is: third region, fourth region, first region, and second region. In some embodiments, there is a linker between any one of the first, second, third and fourth regions. In some embodiments, there is a linker between the first and fourth regions.

In some embodiments, the third and fourth regions are 3′ of the first and second regions. In some embodiments, the order of the regions of the extended guide RNA from 5′ to 3′ is: first region, second region, third region, and fourth region. In some embodiments, there is a linker between the second and third regions. In some embodiments, the effector protein is linked to an RDDP. In some embodiments, the RDDP comprises a reverse transcriptase.

### Protein Modification Activity

In some embodiments, a fusion partner provides enzymatic activity that modifies a protein associated with a target nucleic acid. The protein may be a histone, an RNA binding protein, or a DNA binding protein. Examples of such protein modification activities include: methyltransferase activity, such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity; phosphatase activity; ubiquitin ligase activity; deubiquitinating activity; adenylation activity; deadenylation activity; SUMOylating activity; deSUMOylating activity; ribosylation activity; deribosylation activity; myristoylation activity; and demyristoylation activity.

### CRISPRa Fusions and CRISPRi Fusions

In some embodiments, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, fusion partners that increase or decrease transcription include a transcription activator domain or a transcription repressor domain, respectively.

In some embodiments, fusion partners activate or increase expression of a target nucleic acid. Such fusion proteins comprising the described fusion partners and an effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. In some embodiments, the transcriptional activators may promote transcription by: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. In some embodiments, the fusion partner is a reverse transcriptase.

Non-limiting examples of fusion partners that promote or increase transcription include: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof. Other non-limiting examples of suitable fusion partners include: proteins and protein domains responsible for stimulating translation (e.g., Staufen); proteins and protein domains responsible for (e.g., capable of) modulating translation (e.g., translation factors such as initiation factors, elongation factors, release factors, etc., e.g., eIF4G); proteins and protein domains responsible for stimulation of RNA splicing (e.g., Serine/Arginine-rich (SR) domains); and proteins and protein domains responsible for stimulating transcription (e.g., CDK7 and HIV Tat).

In some embodiments, fusions partners inhibit or reduce expression of a target nucleic acid. Such fusion proteins comprising described fusion partners and an effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. In some embodiments, the transcriptional repressors may inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

Non-limiting examples of fusion partners that decrease or inhibit transcription include: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable fusion partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

In some embodiments, fusion proteins are targeted by a guide nucleic acid (e.g., guide RNA) to a specific location in a target nucleic acid and exert locus-specific regulation such as blocking RNA polymerase binding to a promoter (which selectively inhibits transcription activator function), and/or changes a local chromatin status (e.g., when a fusion sequence is used that edits the target nucleic acid or modifies a protein associated with the target nucleic acid). In some embodiments, the modifications are transient (e.g., transcription repression or activation). In some embodiments, the modifications are inheritable. For example, epigenetic modifications made to a target nucleic acid, or to proteins associated with the target nucleic acid, e.g., nucleosomal histones, in a cell, can be observed in a successive generation.

In some embodiments, fusion partner comprises an RNA splicing factor. The RNA splicing factor may be used (in whole or as fragments thereof) for modular organization, with separate sequence-specific RNA binding modules and splicing effector domains. In some embodiments, the RNA splicing factors comprise members of the Serine/Arginine-rich (SR) protein family containing N-terminal RNA recognition motifs (RRMs) that bind to exonic splicing enhancers (ESEs) in pre-mRNAs and C-terminal RS domains that promote exon inclusion. In some embodiments, a hnRNP protein hnRNP A1 binds to exonic splicing silencers (ESSs) through its RRM domains and inhibits exon inclusion through a C-terminal Glycine-rich domain. In some embodiments, the RNA splicing factors may regulate alternative use of splice site (ss) by binding to regulatory sequences between two alternative sites. For example, in some embodiments, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. Long splicing isoform Bcl-xL is a potent apoptosis inhibitor expressed in long-lived postmitotic cells and is up-regulated in many cancer cells, protecting cells against apoptotic signals. Short isoform Bcl-xS is a pro-apoptotic isoform and expressed at high levels in cells with a high turnover rate (e.g., developing lymphocytes). A ratio of the two Bcl-x splicing isoforms is regulated by multiple c\acute{ω}-elements that are located in either core exon region or exon extension region (i.e., between the two alternative 5′ splice sites). For more examples, see WO2010075303, which is hereby incorporated by reference in its entirety.

### Recombinases

In some embodiments, fusion partners comprise a recombinase. In some embodiments, effector proteins described herein are linked with the recombinase. In some embodiments, the effector proteins have reduced nuclease activity or no nuclease activity. In some embodiments, the recombinase is a site-specific recombinase.

In some embodiments, a catalytically inactive effector protein is linked with a recombinase, wherein the recombinase can be a site-specific recombinase. Such polypeptides can be used for site-directed transgene insertion. Non-limiting examples of site-specific recombinases include a tyrosine recombinase (e.g., Cre, Flp or lambda integrase), a serine recombinase (e.g., gamma-delta resolvase, Tn3 resolvase, Sin resolvase, Gin invertase, Hin invertase, Tn5044 resolvase, IS607 transposase and integrase), or mutants or variants thereof. In some embodiments, the recombinase is a serine recombinase. Non-limiting examples of serine recombinases include gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include:Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety. In some embodiments, the fusion protein comprises a linker that links the recombinase to the Cas-CRISPR domain of the effector protein. In some embodiments, the linker is The-Ser.

### 4. Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

In general guide nucleic acids are characterized by a nucleotide sequence. Such nucleotide sequence may be described as a nucleotide sequence of either DNA or RNA. In some embodiments, a guide nucleic acid comprises a ribonucleotide with a thymine nucleobase. However, no matter the form the sequence is described, it is readily understood that such nucleotide sequences can be revised to be RNA or DNA, as needed, for describing a sequence within a guide nucleic acid itself or the sequence that encodes a guide nucleic acid. Similarly, disclosure of the nucleotide sequences described herein includes a complementary nucleotide sequence, a reverse nucleotide sequence, and the reverse complement nucleotide sequence, any one of which can be a nucleotide sequence for use in a guide nucleic acid. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure. In some embodiments, uridines can be exchanged for pseudouridines (e.g., 1N-Methyl-Pseudouridine). In some embodiments, all uridines can be exchanged for 1N-Methyl-Pseudouridine. In this application, U can represent uracil or 1N-Methyl-Pseudouridine. A guide nucleic acid may be chemically synthesized or recombinantly produced by any suitable methods. Guide nucleic acids and portions thereof may be found in or identified from a CRISPR array present in the genome of a host organism or cell.

In some embodiments, the guide nucleic acid comprises a non-natural nucleobase sequence. In some embodiments, the non-natural sequence is a nucleobase sequence that is not found in nature. The non-natural sequence may comprise a portion of a naturally-occurring sequence, wherein the portion of the naturally-occurring sequence is not present in nature absent the remainder of the naturally-occurring sequence. In some embodiments, the nucleotide sequence of the guide nucleic acid is not found in nature. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, a guide nucleic acid may comprise a repeat sequence, an intermediary sequence and a spacer sequence or a crRNA wherein the crRNA comprises a repeat sequence and a spacer sequence coupled by a linker sequence. In some embodiments, the guide nucleic acid comprises two heterologous sequences arranged in an order or proximity that is not observed in nature. Therefore, guide nucleic acid compositions described herein are not naturally occurring.

In general, a guide nucleic acid comprises a first nucleotide sequence that is capable of being non-covalently bound by an effector protein and a second nucleotide sequence that hybridizes to a target nucleic acid. In some embodiments, the first nucleotide sequence is located 5′ to second nucleotide sequence. In some embodiments, second nucleotide sequence is located 5′ to first nucleotide sequence. In some embodiments, the first nucleotide sequence comprises an intermediary sequence. In some embodiments, the first nucleotide sequence comprises a repeat sequence. In some embodiments, an effector protein binds to at least a portion of the first nucleotide sequence. In some embodiments, the second nucleotide sequence comprises a spacer sequence, wherein the spacer sequence can interact in a sequence-specific manner with (e.g., has complementarity with, or can hybridize to a target sequence in) a target nucleic acid (e.g., a target nucleic acid in the NRAS or KRAS gene or a mutated version thereof). Although the term may imply that a gRNA consists of RNA, in some embodiments a gRNA may comprise one or more deoxyribonucleotides and/or a deoxyribonucleotide nucleobase (e.g., thymine). However, the majority of the nucleotides in a guide nucleic acid (at least 50%) are ribonucleotides.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid (e.g., a target sequence in the NRAS or KRAS gene or a mutated version thereof). For example, when a guide nucleic acid and an effector protein are complexed to form an RNP, at least a portion of the guide nucleic acid hybridizes to a target sequence in a target nucleic acid (e.g., the NRAS or KRAS gene or a mutated version thereof). Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, the compositions, systems, and methods of the present disclosure comprise two or more guide nucleic acids (e.g., 2, 3, 4, 5, 6, 7, 9, 10 or more guide nucleic acids), and/or uses thereof. Multiple guide nucleic acids may target an effector protein to different locations in the target nucleic acid by hybridizing to different target sequences. In some embodiments, a first guide nucleic acid may hybridize within a location of the target nucleic acid that is different from where a second guide nucleic acid may hybridize the target nucleic acid. In some embodiments, the first loci and the second loci of the target nucleic acid may be located at least 1, at least 10, at least 20, at least 30, at least 40, at least 50, at least 60, at least 70, at least 80, at least 90 or at least 100 nucleotides apart. In some embodiments, the first loci and the second loci of the target nucleic acid may be located between 100 and 200, 200 and 300, 300 and 400, 400 and 500, 500 and 600, 600 and 700, 700 and 800, 800 and 900 or 900 and 1000 nucleotides apart.

In some embodiments, the first loci and/or the second loci of the target nucleic acid are located in an intron of a gene. In some embodiments, the first loci and/or the second loci of the target nucleic acid are located in an exon of a gene. In some embodiments, the first portion and/or the second portion of the target nucleic acid are located on either side of an exon and cutting at both sites results in deletion of the exon. In some embodiments, composition, systems, and methods comprise a donor nucleic acid that may be inserted in replacement of a deleted or cleaved sequence of the target nucleic acid. In some embodiments, compositions, systems, and methods comprising multiple guide nucleic acids or uses thereof comprise multiple effector proteins, wherein the effector proteins may be identical, non-identical, or combinations thereof.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, the guide nucleic acid comprises 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least 10, 15, 20, 25, 30, 35, 40, 45, 50, 55, 60 linked nucleotides. In some embodiments, a guide nucleic acid comprises at least 25 linked nucleotides. A guide nucleic acid may comprise 10 to 50 linked nucleotides. In some embodiments, the guide nucleic acid comprises or consists essentially of about 12 to about 80 linked nucleotides, about 12 to about 50, about 12 to about 45, about 12 to about 40, about 12 to about 35, about 12 to about 30, about 12 to about 25, from about 12 to about 20, about 12 to about 19, about 19 to about 20, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleotides. In some embodiments, the guide nucleic acid comprises about 10 to about 60, about 20 to about 50, or about 30 to about 40 linked nucleotides.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

Guide nucleic acids may comprise deoxyribonucleotides, ribonucleotides or a combination thereof. In some embodiments, a guide nucleic acid comprises a ribonucleotide with a thymine nucleobase. Guide nucleic acids may comprise a chemically modified nucleobase or phosphate backbone. Guide nucleic acids may be referred to herein as a guide RNA (gRNA). However, a guide RNA is not limited to ribonucleotides, but may comprise deoxyribonucleotides and other chemically modified nucleotides. A guide nucleic acid may comprise a non-naturally occurring guide nucleic acid, including a guide nucleic acid that is designed to contain a chemical or biochemical modification.

In some embodiments, effector proteins are targeted by a guide nucleic acid (e.g., a guide RNA) to a specific location in the target nucleic acid where they exert locus-specific nucleotide modification or gene regulation. Non-limiting examples of gene regulation include blocking RNA polymerase binding to a promoter (which selectively inhibits transcription activator function), and/or modifying local chromatin (e.g., modifying the target nucleic acid or modifying a protein associated with the target nucleic acid). The guide RNA may bind to a target nucleic acid (e.g., a single strand of a target nucleic acid) or a portion thereof, an amplicon thereof, or a portion thereof. By way of non-limiting example, a guide nucleic acid may bind to a portion of a gene associated with a genetic disorder, or an amplicon thereof, as described herein.

In some embodiments, the compositions, systems, and methods of the present disclosure may comprise an additional guide nucleic acid or a use thereof. An additional guide nucleic acid can target an effector protein to a different location in the target nucleic acid by binding to a different portion of the target nucleic acid from the first guide nucleic acid. A system in which two different guide nucleic acids are used to target two different locations in the target nucleic acid may be referred to as a dual guided system. In certain embodiments, upon removal of a sequence between two guide nucleic acids, the wild-type reading frame may be restored, e.g., by a polymerase, resulting in at least a partially functional protein.

### Spacer Sequences

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

The spacer sequence of a guide nucleic acid is complementary to a target sequence of a target nucleic acid. The spacer sequence of a guide nucleic acid may be at least 70%, at least 75%, at least 80%, at least 85%, at least 90%, at least 95%, or 100% complementary to a target sequence of a target nucleic acid. In general, the spacer sequence is capable of hybridizing to a target sequence of a target nucleic acid. It is understood that the spacer sequence need not be 100% complementary to that of a target sequence of a target nucleic acid to hybridize or hybridize specifically to the target sequence.

In some embodiments, the spacer region is 5-50 linked nucleotides in length. In some embodiments, the spacer region is 15-28 linked nucleotides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides in length. In some embodiments, the spacer region is 18-24 linked nucleotides in length. In some embodiments, the spacer region is at least 15 linked nucleotides in length. In some embodiments, the spacer region is at least 16, 18, 20, or 22 linked nucleotides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleotides in length. In some embodiments, the spacer region is at least 18 linked nucleotides in length. In some embodiments, the spacer region is at least 20 linked nucleotides in length. In some embodiments, the spacer region is at least 80%, at least 85%, at least 90%, at least 95% or 100% complementary to a target sequence of the target nucleic acid. In some embodiments, the spacer region is 100% complementary to the target sequence of the target nucleic acid. In some embodiments, the spacer region comprises at least 15 contiguous nucleobases that are complementary to the target nucleic acid.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing to an equal length portion of a target nucleic acid (e.g., a target sequence). In some embodiments, a spacer sequence comprises a sequence that is at least 65%, at least 70%, at least 75%, at least 80%, at least 85%, at least 90%, at least 95% or 100% complementary to a target sequence of a target nucleic acid. In some embodiments, a spacer sequence comprises a sequence that is at least 65%, at least 70%, at least 75%, at least 80%, at least 85%, at least 90%, at least 95% or 100% complementary to a target sequence in a mutated version of a target nucleic acid. In some embodiments, the spacer sequence comprises at least 10, at least 11, at least 12, at least 13, at least 14, at least 15, at least 16, at least 17, at least 18, at least 19, or at least 20, contiguous nucleotides that are capable of hybridizing to the target sequence. In some embodiments, the spacer sequence comprises at least 10, at least 11, at least 12, at least 13, at least 14, at least 15, at least 16, at least 17, at least 18, at least 19, or at least 20 contiguous nucleotides that are complementary to the target sequence.

In some embodiments, a spacer sequence comprises a nucleotide sequence that is complementary to a target sequence comprising one or more mutations relative to a wildtype sequence of a target nucleic acid. In some embodiments, a spacer sequence comprises a nucleotide sequence that is complementary to a target sequence that is adjacent to a mutated protospacer adjacent motif (PAM) of a target nucleic acid. In some embodiments, guide nucleic acids comprising such spacer sequences, when combined with an effector protein described herein, direct the selective modification (e.g., DNA strand cleavage, insertion, deletion, mutation of one or more nucleic acids) of a mutant allele comprising the one or more mutations or the mutated PAM sequence. In some embodiments, the guide nucleic acids comprising such spacer sequences do not result in modification of the wildtype allele.

TABLE 10 provides illustrative spacer sequences for use with the compositions, systems, and methods of the disclosure. In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to a sequence as set forth in TABLE 10. In some embodiments, spacer sequences comprise at least 10, at least 11, at least 12, at least 13, at least 14, at least 15, at least 16, at least 17, or at least 18 contiguous nucleotides of TABLE 10.

In some embodiments, the spacer sequence comprises one or more nucleobase alterations at one or more positions in any one of the sequences of TABLE 10. Alternative nucleobases can be any one or more of A, C, G, T or U, or a deletion, or an insertion. By way of non-limiting example, a guanine nucleobase could be replaced with the nucleobase of any one of a cytosine, adenosine, thymine, and uracil. In some instance, the spacer sequence comprises only one nucleobase alterations relative to a sequence of TABLE 10. In some instance, the spacer sequence comprises not more than 1, not more than 2, nor more than 3, or not more than 4 nucleobase alterations relative to a sequence of TABLE 10.

### Repeat Sequences

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence includes a nucleotide sequence that is capable of forming a guide nucleic acid-effector protein complex (e.g., a RNP complex). In some embodiments, the repeat sequence may also be referred to as a “protein-binding segment.”

In some embodiments, a repeat sequence is adjacent to a spacer sequence. In some embodiments, a repeat sequence is followed by a spacer sequence in the 5′ to 3′ direction. In some embodiments, a repeat sequence is adjacent to an intermediary RNA sequence. In some embodiments, a repeat sequence is 3′ to an intermediary RNA sequence. In some embodiments, an intermediary RNA sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., at least 65%, at least 70%, at least 75%, at least 80%, at least 85%, at least 90%, at least 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

TABLE 11 provides illustrative repeat sequences for use with the compositions and methods of the disclosure. In some embodiments, the repeat sequence comprises at least 70%, at least 80%, at least 90%, at least 91%, at least 92%, at least 93%, at least 94%, at least 95%, at least 96%, at least 97%, at least 98%, at least 99%, or 100% sequence identity to any one of the sequences as set forth in TABLE 11.

In some embodiments, compositions, systems, and methods described herein comprise a sequence that is at least 65%, at least 70%, at least 80%, at least 90%, at least 92%, at least 95%, at least 97%, or at least 99%, or 100% identical to any one of the sequences as set forth in TABLE 11.

In some embodiments, guide nucleic acids comprise a spacer sequence that is at least 80%, at least 85%, at least 80%, at least 85%, at least 90%, at least 95%, or 100% identical to any one of the sequences as set forth in TABLE 10; and a repeat sequence that is at least 80%, at least 85%, at least 80%, at least 85%, at least 90%, at least 95%, or 100% identical to any one of the sequences of TABLE 11.

In some embodiments, guide nucleic acids described herein comprise a spacer sequence that is at least 80%, at least 85%, at least 80%, at least 85%, at least 90%, at least 95%, or 100% identical to any one of SEQ ID NOs: 72-75 and a repeat sequence that is at least 80%, at least 85%, at least 80%, at least 85%, at least 90%, at least 95%, or 100% identical to SEQ ID NOs: 81-88.

### Intermediary Sequences

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments the intermediary sequence forms a secondary structure, for example in a cell, and an effector protein binds the secondary structure. In some embodiments the intermediary sequence is

(SEQ ID NO: 91)
ACAGCUUAUUUGGAAGCUGAAAUGUGAGGUUUAUAACACUCACAAGAAUC
CU

In some embodiments, the length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

In some embodiments, the intermediary sequence is at least 70%, at least 80%, at least 90%, at least 92%, at least 95%, at least 97%, or at least 99%, or 100% identical to SEQ ID NO: 91.

In some embodiments, an intermediary sequence comprises at least 5, at least 6, at least 7, at least 8, at least 9, at least 10, at least 20, at least 30, at least 40, at least 45, or at least 50 contiguous nucleotides of any one of SEQ ID NO: 91. Such an intermediary sequence may be useful in a guide nucleic acid that is to be used with an effector protein that is at least 80%, at least 85%, at least 90%, at least 95%, at least 98%, at least 99% or 100% identical to any of SEQ ID NOs: 3, 4, and 44-61.

### Handle Sequence

In some embodiments, compositions, systems and methods described herein comprise the nucleic acid, wherein the nucleic acid comprises a handle sequence. In some embodiments, the handle sequence comprises an intermediary sequence. In some embodiments, the intermediary sequence is at the 3′-end of the handle sequence. In some embodiments, the intermediary sequence is at the 5′-end of the handle sequence. In some embodiments, the handle sequence further comprises one or more of linkers and repeat sequences. In some embodiments, the linker comprises a sequence of 5′-GAAA-3′ (SEQ ID NO: 92). In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker.

In some embodiments, an sgRNA may include a handle sequence having a hairpin region, as well as a linker and a repeat sequence. The sgRNA having a handle sequence can have a hairpin region positioned 3′ of the linker and/or repeat sequence. The sgRNA having a handle sequence can have a hairpin region positioned 5′ of the linker and/or repeat sequence. The hairpin region may include a first sequence, a second sequence that is reverse complementary to the first sequence, and a stem-loop linking the first sequence and the second sequence.

In some embodiments, an effector protein may recognize a secondary structure of a handle sequence. In some embodiments, at least a portion of the handle sequence interacts with an effector protein described herein. Accordingly, in some embodiments, at least a portion of the intermediary sequence interacts with the effector protein described herein. In some embodiments, both, at least a portion of the intermediary sequence and at least a portion of the repeat sequence, interacts with the effector protein. In general, the handle sequence is capable of interacting (e.g., non-covalent binding) with any one of the effector proteins described herein.

In some embodiments, the handle sequence of a sgRNA comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the sgRNA comprises a pseudoknot (e.g., a secondary structure comprising a stem at least partially hybridized to a second stem or half-stem secondary structure). An effector protein may recognize a sgRNA comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the sgRNA comprises at least 2, at least 3, at least 4, or at least 5 stem regions.

A handle sequence may include deoxyribonucleosides, ribonucleosides, chemically modified nucleosides, or any combination thereof. In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, the length of a handle sequence in a sgRNA is not greater than 50, 56, 66, 67, 68, 69, 70, 71, 72, 73, 95, or 105 linked nucleotides. In some embodiments, the length of a handle sequence in a sgRNA is about 30 to about 120 linked nucleotides. In some embodiments, the length of a handle sequence in a sgRNA is about 50 to about 105, about 50 to about 95, about 50 to about 73, about 50 to about 71, about 50 to about 70, or about 50 to about 69 linked nucleotides. In some embodiments, the length of a handle sequence in a sgRNA is 56 to 105 linked nucleotides, from 56 to 105 linked nucleotides, 66 to 105 linked nucleotides, 67 to 105 linked nucleotides, 68 to 105 linked nucleotides, 69 to 105 linked nucleotides, 70 to 105 linked nucleotides, 71 to 105 linked nucleotides, 72 to 105 linked nucleotides, 73 to 105 linked nucleotides, or 95 to 105 linked nucleotides. In some embodiments, the length of a handle sequence in a sgRNA is 40 to 70 nucleotides. In some embodiments, the length of a handle sequence in a sgRNA is 50, 56, 66, 67, 68, 69, 70, 71, 72, 73, 95, or 105 linked nucleotides.

In some embodiments, a handle sequence comprises a nucleotide sequence that is at least 65%, at least 70%, at least 75%, at least 80%, at least 85%, at least 90%, at least 95%, at least 97%, at least 98%, or at least 99%, or 100% identical the sequence:

(SEQ ID NO: 90)
ACAGCUUAUUUGGAAGCUGAAAUGUGAGGUUUAUAACACUCACAAGAAUC
CUGAAAAAGGAUGCCAAAC

### Guide Nucleic Acid Modification

In some embodiments, a guide nucleic acid comprises one or more modifications. Nonlimiting examples of modifications include a nucleobase modification and a backbone modification. Modification may provide the nucleic acid with a new or enhanced feature, e.g., improved stability or increased activity. In general, a guide nucleic acid comprising one or more modifications is synthesized to comprise the one or more modifications and thus, it is not naturally occurring.

Exemplary nucleic acid modifications include but are not limited to: 2′ O-methyl modified nucleotides, 2′-fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). The phosphorothioate (PS) bond (i.e., a phosphorothioate linkage) substitutes a sulfur atom for a non-bridging oxygen in the phosphate backbone of a nucleic acid (e.g., an oligo). The normal linkage or backbone of RNA and DNA is a 3′ to 5′ phosphodiester linkage. This modification may render the guide nucleic acid more resistant to nuclease degradation relative to a guide nucleic acid with the same sequence but without the PS linkage. In some embodiments, PS linkages occur between any of the 5′-most and 3′-most 3-5 nucleotides of the guide nucleic acid.

In some embodiments, a subject nucleic acid has one or more nucleotides that are 2′ O-methyl modified nucleotides. In some embodiments, the 2′ O-methyl occurs on any of the 5′-most and 3′ most 3-5 nucleotides of the guide nucleic acid. In some embodiments, the guide nucleic acid comprises one or more 2′-fluoro modified nucleotides. In some embodiments, the guide nucleic acid comprises one or more LNA bases. In some embodiments, the guide nucleic acid comprises a 5′ cap (e.g., a 7-methylguanylate cap (m7G)). In some embodiments, a guide nucleic acid comprises a combination of modified nucleotides.

Guide nucleic acids may include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH3, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O— CH₂CH₂OCH₃, also known as 2′-O-(2-methoxyethyl) or 2′-MOE). A further suitable modification includes 2′-dimethylaminooxy ethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂. Other suitable sugar substituent groups include methoxy (—O—CH₃), aminopropoxy (—OCH₂CH₂CH₂NH₂), allyl (—CH₂—CH═CH₂), —O-allyl (—O—CH₂—CH═CH2) and fluoro (F).

In some embodiments, the guide nucleic acid comprises a modification of its 5′-most nucleotide. In some embodiments, the guide nucleic acid comprises a modification of its 3′-most nucleotide. In some embodiments, the guide nucleic acid comprises a modification of its 5′-most nucleotide and its 3′-most nucleotide. In some embodiments, the guide nucleic acid comprises modifications of its 1, 2, 3, or 4 5′-most nucleotides. In some embodiments, the guide nucleic acid comprises modifications of its 1, 2, 3 or 4 3′-most nucleotides. In some embodiments, the guide nucleic acid comprises modification of its 1, 2, 3, or 4 5′-most nucleotides and its 1, 2, 3, or 4 3′-most nucleotides. In some embodiments, at least one of the modifications is a 2′ O-methyl modification. In some embodiments, all of the modifications are 2′ O-methyl modifications.

In some embodiments, the guide nucleic acid comprises a phosphorothioate linkage between its two 5′-most nucleotides. In some embodiments, the guide nucleic acid comprises a phosphorothioate linkage between its two 3′-most nucleotides. In some embodiments, the guide nucleic acid comprises a phosphorothioate linkage between its two 5′-most nucleotides, and a second phosphorothioate linkage between its two 3′-most nucleotides. In some embodiments, the guide nucleic acid comprises phosphorothioate linkages between its 1, 2, 3 or 4 of its 5′-most nucleotides. In some embodiments, the guide nucleic acid comprises phosphorothioate linkages between 1, 2, 3 or 4 of its 3′-most nucleotides. In some embodiments, the guide nucleic acid comprises phosphorothioate linkages between 1, 2, 3, or 4 of its 5′-most nucleotides and between 1, 2, 3, or 4 of its 3′-most nucleotides.

In some embodiments, the sequences in any of TABLES 10-13 and SEQ ID NOs: 90-92 can be modified. In some embodiments, the modification includes at least one phosphorothioate (PS) linkage. In some embodiments, the modification includes at least one 2′-O-Methyl oligonucleotide (OMe). In some embodiments, the modification includes at least one locked nucleic acid (LNA). In some embodiments, the modification includes at least one phosphorodiamidate morpholino oligonucleotide (PMO). In some embodiments, the modification includes at least one or more peptide nucleic acid (PNA). In some embodiments, the first 3 and last 3 amino acids are O-Me modified, and the first 3 and last 2 linkages are phosphorothioate linkages. In some embodiments, the modification comprises 2′ O-Methyl modifications of the first 3 nucleotides and last 3 nucleotides of the guide nucleic acid sequence, phosphorothioate linkages between the first 4 nucleotides of the guide nucleic acid sequence, and phosphorothioate linkages between the last 3 nucleotides of the guide nucleic acid sequence. In some embodiments, the sequence is modified mN*mN*mN*NNN . . . NNNmN*mN*mN where m is a 2′ O-Me modified sugar moiety and the * denotes a PS linkage.

### Nucleic Acid Linkers

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises two or more linkers. In some embodiments, at least two or more linkers are the same. In some embodiments, at least two or more linkers are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′ (SEQ ID NO: 92).

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Single Nucleic Acid Systems

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more effector proteins described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

In some embodiments, guide nucleic acid comprises a crRNA comprising a spacer sequence(s) and a repeat sequence(s) present within the same polynucleotide molecule. In some embodiments, the spacer sequence is adjacent to the repeat sequence. In some embodiments, the spacer sequence follows the repeat sequence in a 5′ to 3′ direction. In some embodiments, the spacer sequence precedes the repeat sequence in a 5′ to 3′ direction. In some embodiments, the spacer(s) and repeat sequence(s) are linked directly to one another. In some embodiments, a linker is present between the spacer(s) and repeat sequence(s). Linkers may be any suitable linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary RNA. In some embodiments, a single nucleic acid system comprises a guide nucleic acid comprising a crRNA and an intermediary RNA.

In some embodiments, a crRNA is sufficient to form complex with an effector protein (e.g., to form an RNP) through the repeat sequence and direct the effector protein to a target nucleic acid sequence through the spacer sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

sgRNA

In some embodiments, a guide nucleic acid comprises a single guide RNA (sgRNA). In some embodiments, the guide nucleic acid is a sgRNA. The combination of a spacer sequence (e.g., a nucleotide sequence that hybridizes to a target sequence in a target nucleic acid) with a handle sequence may be referred to herein as a single guide RNA (sgRNA), wherein the spacer sequence and the handle sequence are covalently linked. In some embodiments, the spacer sequence and handle sequence are linked by a phosphodiester bond. In some embodiments, the spacer sequence and handle sequence are linked by one or more linked nucleotides. In some embodiments, a guide nucleic acid may comprise a spacer sequence, a repeat sequence, a handle sequence, or a combination thereof. In some embodiments, the handle sequence may comprise a portion of, or all of, a repeat sequence. In general, a sgRNA comprises a first region (FR) and a second region (SR), wherein the FR comprises a handle sequence and the SR comprises a spacer sequence.

In some embodiments, the compositions comprise a guide RNA and an effector protein without a tracrRNA (e.g., a single nucleic acid system), wherein the guide RNA is a sgRNA. A sgRNA may include deoxyribonucleosides, ribonucleosides, chemically modified nucleosides, or any combination thereof. A sgRNA may also include a nucleotide sequence that forms a secondary structure (e.g., one or more hairpin loops) that facilitates the binding of an effector protein to the sgRNA and/or modification activity of an effector protein on a target nucleic acid (e.g., a hairpin region). Such a sequence can be contained within a handle sequence as described herein.

In some embodiments, a sgRNA comprises one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and a crRNA; an intermediary sequence, a repeat sequence, and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g., covalently linked, such as through a phosphodiester bond) In some embodiments, a repeat sequence and a spacer sequence are linked in an sgRNA by any suitable linker, examples of which are provided herein.

An exemplary handle sequence in a sgRNA may comprise, from 5′ to 3′, a 5′ region, a hairpin region, and a 3′ region. In some embodiments, the 5′ region may hybridize to the 3′ region. In some embodiments, the 5′ region does not hybridize to the 3′ region. In some embodiments, the 3′ region is covalently linked to a spacer sequence (e.g., through a phosphodiester bond). In some embodiments, the 5′ region is covalently linked to a spacer sequence (e.g., through a phosphodiester bond).

### Dual Nucleic Acid System

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, in a dual nucleic acid system, activity of the effector protein requires binding to a tracrRNA molecule.

In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### Exemplary Guide RNAs

In some embodiments, the guide nucleic acids disclosed herein comprise a spacer sequence that is at least 70%, at least 75%, at least 80%, at least 85%, at least 90%, at least 95%, or 100% identical to any one of SEQ ID NOs: 72-75 and a repeat sequence that is at least 70%, at least 75%, at least 80%, at least 85%, at least 90%, at least 95%, or 100% identical to any one SEQ ID NOs: 81-88. In some embodiments, the guide nucleic acids disclosed herein comprise a spacer sequence consisting of a sequence selected from the group consisting of SEQ ID NOs: 72-75 and a repeat sequence consisting of a sequence selected from the group consisting of SEQ ID NOs: 81-88.

In some embodiments, the guide nucleic acids disclosed herein comprise a nucleic acid sequence that is at least 70%, at least 75%, at least 80%, at least 85%, at least 90%, at least 95%, at least 96%, at least 97%, at least 98%, at least 99%, or 100% identical to any one of SEQ ID NOs: 99-102. In some embodiments, the guide nucleic acids disclosed herein consist of a sequence selected from the group consisting of SEQ ID NOs: 99-102.

In some embodiments, the guide nucleic acids provided herein comprise a spacer sequence that is complementary to a target sequence in the mutant or wildtype allele of the NRAS gene.

In some embodiments, the guide nucleic acids provided herein comprise a spacer sequence that is complementary to a target sequence in the mutant or wildtype allele of the KRAS gene.

Exemplary guide nucleic acids sequences useful for systems, compositions, and methods disclosed herein are provided in TABLE 12. In some embodiments, the guide nucleic acid comprises a sequence that is at least 70%, at least 75%, at least 80%, at least 85%, at least 90%, at least 95%, or 100% identical to any one of the sequences of TABLE 12. In some embodiments, the guide nucleic acid consists of a sequence that is at least 70%, at least 75%, at least 80%, at least 85%, at least 90%, at least 95%, or 100% identical to any one of the sequences of TABLE 12. In some embodiments, the guide nucleic acids provided in TABLE 12 comprise an additional “G” at the 5′ end of the sequence.

The exemplary guide nucleic acids shown in TABLE 12 comprise a 20nt repeat sequence (SEQ ID NO: 86). However, it should be understood that these guides can comprise any of the repeat sequences disclosed herein (e.g., any one of SEQ ID NOs: 81-88). For example, in some embodiments, the guide sequence comprises a spacer sequence of any one of SEQ IDs: 72-75 with the repeat sequence of SEQ ID NO: 81.

In some embodiments, guide nucleic acids comprise a portion or all of a sequence as set forth in any one of TABLES 10, 11 and 12. In some embodiments, a guide nucleic acid comprises at least 9, at least 10, at least 11, at least 12 contiguous nucleotides of any one of SEQ ID NOs: 72-75, 81-88, and 99-102. In some embodiments, the guide nucleic acid comprises at least 15, at least 20, at least 25, at least 30, or at least 35 contiguous nucleotides of any one of SEQ ID NOs: 72-75, 81-88, and 99-102.

In some embodiments, compositions, systems, and methods disclosed herein comprise a spacer sequence that is at least 70%, at least 75%, at least 80%, at least 85%, at least 90%, at least 95%, or 100% identical to any one of the sequences as set forth in TABLE 10, and comprise a repeat sequence that is at least 70%, at least 75%, at least 80%, at least 85%, at least 90%, at least 95%, or 100% identical to any one of the sequences of TABLE 11.

In some embodiments, compositions, systems, and methods described herein comprise a guide nucleic acid comprising a sequence that is at least 65%, at least 70%, at least 75%, at least 80%, at least 85, at least 90%, at least 92%, at least 95%, at least 97%, or at least 99%, or 100% identical to any one of the sequences as set forth in TABLE 12.

In some embodiments, the guide nucleic acids disclosed herein comprise a spacer sequence that is at least 70%, at least 75%, at least 80%, at least 85%, at least 90%, at least 95%, at least 96%, at least 97%, at least 98%, at least 99%, or 100% identical to any one of SEQ ID NOs: 77, 78 and 80 and a repeat sequence that is at least 70%, at least 75%, at least 80%, at least 85%, at least 90%, at least 95%, at least 96%, at least 97%, at least 98%, at least 99%, or 100% identical to SEQ ID NO: 89. In some embodiments, the guide nucleic acids disclosed herein comprise a spacer sequence consisting of a sequence selected from the group consisting of SEQ ID NOs: 77, 78 and 80 and a repeat sequence consisting of SEQ ID NO: 89.

In some embodiments, the guide nucleic acids further comprise an intermediary sequence that is at least 70%, at least 75%, at least 80%, at least 85%, at least 90%, at least 95%, at least 96%, at least 97%, at least 98%, at least 99%, or 100% identical to SEQ ID NO: 91. In some embodiments, the guide nucleic acids further comprise a handle sequence that is at least 70%, at least 75%, at least 80%, at least 85%, at least 90%, at least 95%, at least 96%, at least 97%, at least 98%, at least 99%, or 100% identical to SEQ ID NO: 90.

In some embodiments, the guide nucleic acids disclosed herein comprise a nucleic acid sequence that is at least 70%, at least 75%, at least 80%, at least 85%, at least 90%, at least 95%, at least 96%, at least 97%, at least 98%, at least 99%, or 100% identical to any one of SEQ ID NOs: 104, 105 and 107. In some embodiments, the guide nucleic acids disclosed herein consist of a sequence selected from the group consisting of SEQ ID NOs: 104, 105 and 107.

Exemplary guide nucleic acids sequences useful for systems, compositions, and methods disclosed herein are provided in TABLE 13. In some embodiments, the guide nucleic acid comprises a sequence that is at least 70%, at least 75%, at least 80%, at least 85%, at least 90%, at least 95%, or 100% identical to any one of the sequences of TABLE 13. In some embodiments, the guide nucleic acid consists of a sequence that is at least 70%, at least 75%, at least 80%, at least 85%, at least 90%, at least 95%, or 100% identical to any one of the sequences of TABLE 13. In some embodiments, the guide nucleic acids provided in TABLE 13 comprise an additional “G” at the 5′ end of the sequence.

In some embodiments, guide nucleic acids comprise a portion or all of a sequence as set forth in TABLES 10, 11 and 13 and SEQ ID NOs: 90-92. In some embodiments, guide nucleic acids comprise at least 60, at least 65, at last 70, at least 75, at least 80, at least 85, at least 86, at least 87, at least 88, or at least 89 contiguous nucleotides of a sequence selected from any of SEQ ID NOs: 77, 78, 80, 89-92, 104, 105 and 107.

In some embodiments, compositions, systems, and methods disclosed herein comprise a spacer sequence that is at least 70%, at least 75%, at least 80%, at least 85%, at least 90%, at least 95%, or 100% identical to any one of the sequences as set forth in TABLE 10, and comprise a handle sequence that is at least 70%, at least 75%, at least 80%, at least 85%, at least 90%, at least 95%, or 100% identical to any one of the sequences of SEQ ID NO: 90.

In some embodiments, compositions, systems, and methods described herein comprise a guide nucleic acid comprising a sequence that is at least 65%, at least 70%, at least 75%, at least 80%, at least 85, at least 90%, at least 92%, at least 95%, at least 97%, or at least 99%, or 100% identical to any one of the sequences as set forth in TABLE 13.

In some embodiments the sequences in any of TABLES 10-13 and SEQ ID NOs: 90-92 can be modified.

In some embodiments the modification includes at least one phosphorothioate (PS) linkage. In some embodiments the modification includes at least one 2′-O-Methyl oligonucleotide (oMe). In some embodiments the modification includes at least one locked nucleic acid (LNA). In some embodiments the modification includes at least one phosphorodiamidate morpholino oligonucleotide (PMO). In some embodiments the modification includes at least one or more peptide nucleic acid (PNA). In some embodiments, the first 3 and last 3 amino acids are 0-Me modified, and the first 3 and last 2 linkages are phosphorothioate linkages. In some embodiments, the sequence is modified mN*mN*mN*NNN . . . NNNmN*mN*mN where m is a 2′ O-Me modified sugar moiety and the * denotes a PS linkage. TABLES 14-15 provide exemplary modified guide nucleic acids.

### 5. Exemplary Systems

In some embodiments, the present disclosure provides a system comprising a guide RNA and an effector protein or fusion protein thereof.

In some embodiments, the system comprises an effector protein comprising an amino acid sequence that is at least 90%, at least 95%, or 100% identical to any one of the sequences recited in TABLE 1 and TABLE 4, and the guide RNA comprises a repeat sequence that is at least 90%, at least 95%, or 100% identical to any one of SEQ ID NOs: 81-88 and a spacer sequence that is at least 90%, at least 95%, or 100% identical to any one of SEQ ID NOs: 72-75. In some embodiments, the effector protein comprises an amino acid that is at least 90%, at least 95%, or 100% identical to SEQ ID NO: 1. In some embodiments, the effector protein comprises an amino acid substitution relative to SEQ ID NO: 1 selected from the group consisting of L26R, E109R, H208R, K184R, K38R, L182R, Q183R, S108R, S198R, and T114R. In some embodiments, the effector protein is a dCas protein. In some embodiments, the dCas protein comprises an amino acid substation of D369A, D369N, D658A, D658N, E567A, and E567Q relative to SEQ ID NO: 1.

In some embodiments, the system comprises an effector protein comprising an amino acid sequence that is at least 90%, at least 95%, or 100% identical to any one of the sequences recited in TABLE 1 and TABLE 6, and the guide RNA comprises a handle sequence that is at least 90%, at least 95%, or 100% identical to SEQ ID NO: 90 and a spacer sequence that is at least 90%, at least 95%, or 100% identical to any one of SEQ ID NOs: 77, 78 and 80. In some embodiments, the guide RNA further comprises an intermediary sequence that is at least 90%, at least 95%, or 100% identical to SEQ ID NO: 90. In some embodiments, the effector protein comprises an amino acid that is at least 90%, at least 95%, or 100% identical to SEQ ID NO: 3. In some embodiments, the effector protein comprises an amino acid substitution relative to SEQ ID NO: 3 selected from the group consisting of D220R, N286K, E225K, I80K, S209F, Y315M, N193K, M298L, M295W, A306K, A218K, and K58W. In some embodiments, the effector protein is a dCas protein. In some embodiments, the dCas protein comprises an amino acid substation of E335Q, D237A D418A, D418N, and E335A relative to SEQ ID NO: 3.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 1 or 24 and a guide nucleic acid comprising a spacer sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 72-75. In some embodiments, the system comprises an effector protein comprising SEQ ID NO: 1 or 24 and a guide nucleic acid comprising a spacer sequence selected from SEQ ID NOs: 72-75. In some embodiments, the system comprises an effector protein consisting of SEQ ID NO: 1 or 24 and a guide nucleic acid comprising a spacer sequence consisting of a sequence selected from SEQ ID NOs: 72-75.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 1 or 24 and a guide nucleic acid comprising a sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 99-102. In some embodiments, the system comprises an effector protein comprising SEQ ID NO: 1 or 24 and a guide nucleic acid comprising a sequence selected from SEQ ID NOs: 99-102. In some embodiments, the system comprises an effector protein consisting of SEQ ID NO: 1 or 24 and a guide nucleic acid comprising a sequence consisting of a sequence selected from SEQ ID NOs: 99-102.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 1 or 24 and a guide nucleic acid comprising a sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 111-113. In some embodiments, the system comprises an effector protein comprising SEQ ID NO: 1 or 24 and a guide nucleic acid comprising a sequence selected from SEQ ID NOs: 111-113. In some embodiments, the system comprises an effector protein consisting of SEQ ID NO: 1 or 24 and a guide nucleic acid comprising a sequence consisting of a sequence selected from SEQ ID NOs: 111-113.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 3 and a guide nucleic acid comprising a spacer sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 77, 78, and 80. In some embodiments, the system comprises an effector protein comprising SEQ ID NO: 3 and a guide nucleic acid comprising a spacer sequence selected from SEQ ID NOs: 77, 78, and 80. In some embodiments, the system comprises an effector protein consisting of SEQ ID NO: 3 and a guide nucleic acid comprising a spacer sequence consisting of a sequence selected from SEQ ID NOs: 77, 78, and 80.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 3 and a guide nucleic acid comprising a sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 104, 105 and 107. In some embodiments, the system comprises an effector protein comprising SEQ ID NO: 3 and a guide RNA comprising a sequence selected from SEQ ID NOs: 104, 105 and 107. In some embodiments, the system comprises an effector protein consisting of SEQ ID NO: 3 and a guide RNA comprising a sequence consisting of a sequence selected from SEQ ID NOs: 104, 105 and 107.

In some embodiments, the system comprises an effector protein that is at least 90%, at least 91%, at least 92%, at least 93%, at least 94%, at least 95%, at least 96%, at least 97%, at least 98%, at least 99%, or 100% identical to SEQ ID NO: 3 and a guide nucleic acid comprising a sequence that is at least 90%, at least 91%, at least 92%, at least 93%, at least 94%, at least 95%, at least 96%, at least 97%, at least 98%, at least 99%, or 100% identical to a sequence selected from SEQ ID NOs: 115, 116 and 118. In some embodiments, the system comprises an effector protein comprising SEQ ID NO: 3 and a guide RNA comprising a sequence selected from SEQ ID NOs: 115, 116 and 118. In some embodiments, the system comprises an effector protein consisting of SEQ ID NO: 3 and a guide RNA comprising a sequence consisting of a sequence selected from SEQ ID NOs: 115, 116 and 118.

### 6. Target Nucleic Acids

Disclosed herein are compositions, systems and methods for detecting and/or editing a target nucleic acid (e.g., the NRAS or KRAS gene or a mutated version thereof). In general, guide nucleic acids described herein comprise a sequence that is complementary to and/or hybridizes to a target sequence of the mutated target gene.

### NRAS

Neuroblastoma RAS viral oncogene homolog (NRAS) is part of the MAPK signaling pathway in mammalian cells, responsible for energy conversion and metabolism. A sequence representing a human wildtype allele of NRAS may be found in the NCBI database with gene accession ID is NC_000001.11. A sequence representing human wildtype NRAS mRNA (also a sense strand of human NRAS cDNA) may be found in the NCBI database with accession number NM_002524.5.

The NRAS gene belongs to a class of genes known as oncogenes. When mutated, oncogenes have the potential to cause normal cells to become cancerous. The most commonly mutated isoform of NRAS mutation typically occur at codons 12, 61, or, less frequently, 13, with 15% of cases harboring point mutations. Whereas mutant NRAS(Q61) disrupts the GTPase activity of RAS, locking it in its active conformation, NRAS(G12) and NRAS(G13) mutations affect the Walker A-motif (p-loop) of the protein, thus decreasing its sensitivity to GTPase-accelerating proteins. NRAS Q61L is an oncogenic mutation, which renders the protein constitutively active, and subsequently leads to unchecked proliferation of the cell.

In some embodiments, the target sequence of the NRAS gene may be a portion of the NRAS gene that encodes the NRAS protein with the Q61L mutation. In some embodiments, the target sequence of the NRAS gene is on a mutant allele (e.g., Q61L mutant allele). In some embodiments, the target sequence of the NRAS Q61L mutant allele comprises a mutation at the position 182 (A>T) relative to a wildtype target locus GGACATACTGGATACAGCTGGACAAGAA (SEQ ID NO: 93). In some embodiments, the target sequence of the NRAS Q61L mutant allele comprises GGACATACTGGATACAGCTGGACTAGAA (SEQ ID NO: 94).

In some embodiments, the compositions, systems, and methods disclosed herein are useful for modifying a NRAS allele, as demonstrated in Examples 1-4. In some embodiments, the compositions, systems, and methods modify a first allele of a NRAS gene (e.g., a mutant allele), and do not modify a second allele of a NRAS gene (e.g., a wildtype allele).

In some embodiments, the compositions, systems, and methods provided herein reduce expression of a mutant allele of a NRAS gene in a cell by at least 10%, at least 20%, at least 30%, at least 40%, at least 50%, at least 60%, at least 70%, at least 80%, at least 90%, at least 95%, or at least 99%, relative to expression of the WT allele in the same cell or expression of the mutant allele in a cell that has not been contacted with the compositions or systems. In some embodiments, the compositions, systems and methods provided herein do not reduce expression of a wildtype allele of a NRAS gene in a cell by more than 10%, more than 20%, more than 30%, more than 40%, or more than 50% relative to expression of the wildtype allele in a cell that has not been contacted with the compositions or systems. In some embodiments, the compositions, systems and methods provided herein abolish expression of a mutant NRAS allele (e.g., knockout) and do not abolish expression of a wildtype allele.

In some embodiments, the guide nucleic acid comprises a spacer sequence that is complementary to a target sequence on the NRAS Q61L mutant allele. In some embodiments, the guide nucleic acid comprises 11, 12, 13, 14, 15, 16, 17, 18, 19, or 20 contiguous nucleotides that are complementary to the target sequence of the NRAS Q61L target locus GGACATACTGGATACAGCTGGACTAGAA (SEQ ID NO: 94), which has a mutation at the position 182 (A>T) relative to the sequence of the corresponding NRAS WT target locus GGACATACTGGATACAGCTGGACAAGAA (SEQ ID NO: 93).

In some embodiments, the guide nucleic acid comprises a spacer sequence that is complementary to a target sequence that is adjacent to a mutated protospacer adjacent motif (PAM) in the target nucleic acid. In some embodiments, the guide nucleic acid comprises a spacer sequence that is complementary to a target sequence adjacent to the mutated PAM sequence TCTA in the NRAS Q61L mutant allele. In some embodiments, the guide nucleic acid comprises a spacer sequence that is at least 80%, at least 85%, at least 90%, at least 95%, at least 97%, at least 98%, at least 99%, or 100% identical to the sequence of SEQ ID NO: 78. In some embodiments, the guide nucleic acid comprises a sequence that is at least 80%, at least 85%, at least 90%, at least 95%, at least 97%, at least 98%, at least 99%, or 100% identical to the sequence of SEQ ID NO: 105. In some embodiments, the effector protein capable of targeting the NRAS Q61L mutant allele with the guide nucleic acid specific for the mutated PAM comprises a CasM265466 protein or a variant thereof. In some instances, the effector protein comprises a sequence that is at least 80%, at least 85%, at least 90%, at least 95%, at least 97%, at least 98%, at least 99%, or 100% identical to the sequence of SEQ ID NO: 3.

In some embodiments, the compositions, systems and methods provided herein useful for targeting a mutant NRAS allele comprise an effector protein described herein. In some embodiments, the effector protein comprises a CasPhi.12 protein or a variant thereof. In some instances, the effector protein comprises a sequence that is at least 80%, at least 85%, at least 90%, at least 95%, at least 97%, at least 98%, at least 99%, or 100% identical to the sequence of SEQ ID NO: 1 or SEQ ID NO: 24. In some embodiments, the guide nucleic acid comprises a spacer sequence comprising a sequence that is at least 80%, at least 85%, at least 90%, at least 95%, at least 97%, at least 98%, at least 99%, or 100% identical to a sequence selected from SEQ ID NOs: 72-75. In some embodiments, the guide nucleic acid comprises a sequence that is at least 80%, at least 85%, at least 90%, at least 95%, at least 97%, at least 98%, at least 99%, or 100% identical to a sequence selected from SEQ ID NO: 99-102. In some embodiments, the guide nucleic acid comprises a sequence that is at least 80%, at least 85%, at least 90%, at least 95%, at least 97%, at least 98%, at least 99%, or 100% identical to a sequence selected from SEQ ID NOs: 111-113. In some embodiments, the effector protein comprises a CasM265466 protein or a variant thereof. In some instances, the effector protein comprises a sequence that is at least 80%, at least 85%, at least 90%, at least 95%, at least 97%, at least 98%, at least 99%, or 100% identical to the sequence of SEQ ID NO: 3. In some embodiments, the guide nucleic acid comprises a spacer sequence comprising a sequence that is at least 80%, at least 85%, at least 90%, at least 95%, at least 97%, at least 98%, at least 99%, or 100% identical to a sequence selected from SEQ ID NOs: 77-78. In some embodiments, the guide nucleic acid comprises a sequence that is at least 80%, at least 85%, at least 90%, at least 95%, at least 97%, at least 98%, at least 99%, or 100% identical to a sequence selected from SEQ ID NOs: 104-105. In some embodiments, the guide nucleic acid comprises a sequence that is at least 80%, at least 85%, at least 90%, at least 95%, at least 97%, at least 98%, at least 99%, or 100% identical to a sequence selected from SEQ ID NO: 115-116.

In some embodiments, the compositions, systems and methods provided herein are useful for treating cancer. In some embodiments, the cancer is hepatocellular carcinoma, melanoma, leukemia, skin cancer, colorectal cancer, acute myeloid leukemia (AML), thyroid cancer, liver cancer, lung cancer, neuroblastoma, bladder cancer, or rhabdomyosarcoma. The compositions, systems and methods provided herein may be used to selectively reduce the growth, reduce the viability, induce cell death or arrest the cell cycle of a cell, wherein the cell comprises one or more mutant NRAS alleles.

### KRAS

Kirsten rat sarcoma virus (KRAS) is a GTPase that is involved in cell proliferation checkpoints. A sequence representing a human wildtype allele of KRAS may be found in the NCBI database with gene accession ID is NC_000012.12. A sequence representing human wildtype KRAS mRNA (also a sense strand of human KRAS cDNA) may be found in the NCBI database with accession number NM_001369786.

Mutations in the KRAS gene can result in a protein that is constitutively in its GTP-bound active state, leading to uninhibited proliferation of cells and accumulation of mutations. In some embodiments, a mutant KRAS allele comprises a mutation in exon 2. In some embodiments, the KRAS allele comprises a single nucleotide polymorphism. Common mutations in KRAS include, but are not limited, to KRAS p.G12C-c.34G>T; KRAS p.G 12D-c.35G>A; and KRAS p.G12V-c.35G>T. The KRAS gene is mutated in more than 90% of pancreatic cancers and more than 30% of colon and lung cancers.

In some embodiments, the target sequence of the KRAS gene may be a portion of the KRAS gene that encodes the KRAS protein with the G12D mutation. In some embodiments, the compositions, systems and methods disclosed herein are useful for modifying KRAS mutants by targeting a single nucleotide polymorphism (SNP) in the KRAS gene, as demonstrated in Example 5. In some embodiments, the compositions, systems and methods provided herein modify a first allele of a KRAS gene (e.g., a mutant allele), and do not modify a second allele of a KRAS gene (e.g., a wildtype allele). Such compositions, systems and methods are particularly useful for targeting KRAS mutants because many KRAS mutants are not easily targeted with small molecules due to their lack of drug binding pockets.

In some embodiments, the compositions, systems and methods provided herein reduce expression of a KRAS mutant in a cell by at least 10%, at least 20%, at least 30%, at least 40%, at least 50%, at least 60%, at least 70%, at least 80%, at least 90%, at least 95%, or at least 99%, relative to expression of the wildtype KRAS gene in a cell. In some embodiments, the mutation is a SNP. In some embodiments, the SNP is selected from 34G>T, 35G>A, and 35G>T in the KRAS gene. In some embodiments, the compositions, systems and methods provided herein reduce expression of a mutant allele of a KRAS gene in a cell by at least 10%, at least 20%, at least 30%, at least 40%, at least 50%, at least 60%, at least 70%, at least 80%, at least 90%, at least 95%, or at least 99%, relative to expression of the wildtype allele in the same cell or expression of the mutant allele in a cell that has not been contacted with the composition. In some embodiments, the compositions, systems and methods provided herein do not reduce expression of a wildtype allele of a KRAS gene in a cell by more than 10%, more than 20%, more than 30%, more than 40%, or more than 50% relative to expression of the wildtype allele in a cell that has not been contacted with the composition. In some embodiments, the compositions, systems and methods provided herein abolish expression of a mutant KRAS allele and do not abolish expression of a wildtype allele.

In some embodiments, the guide nucleic acid comprises a spacer sequence that is complementary to a target sequence comprising a SNP in the KRAS gene. In some embodiments, the SNP is selected from 34G>T, 35G>A, and 35G>T in the KRAS gene. In some embodiments, the SNP is 35G>A in the KRAS gene. In some embodiments, the guide nucleic acid comprises a spacer sequence that is complementary to a target sequence on the KRAS G12D mutant allele. In some embodiments, the guide nucleic acid comprises 11, 12, 13, 14, 15, 16, 17, 18, 19, or 20 contiguous nucleotides that are complementary to a target sequence on the KRAS G12D mutant allele.

In some embodiments, the compositions and methods useful for targeting a mutant KRAS allele comprise an effector protein described herein. In some embodiments, the effector protein comprises a CasM265466 protein or a variant thereof. In some instances, the effector protein comprises a sequence that is at least 80%, at least 85%, at least 90%, at least 95%, at least 97%, at least 98%, at least 99%, or 100% identical to the sequence of SEQ ID NO: 3. In some embodiments, the guide nucleic acid comprises a spacer sequence comprising a sequence that is at least 80%, at least 85%, at least 90%, at least 95%, at least 97%, at least 98%, at least 99%, or 100% identical to the sequence of SEQ ID NO: 80. In some embodiments, the guide nucleic acid comprises a sequence that is at least 80%, at least 85%, at least 90%, at least 95%, at least 97%, at least 98%, at least 99%, or 100% identical to the sequence of SEQ ID NO: 107. In some embodiments, the guide nucleic acid comprises a sequence that is at least 80%, at least 85%, at least 90%, at least 95%, at least 97%, at least 98%, at least 99%, or 100% identical to the sequence of SEQ ID NO: 118.

In some embodiments, the compositions, systems and methods provided herein are useful for treating cancer. In some embodiments, the cancer is pancreatic cancer. In some embodiments, the cancer is colon cancer or lung cancer. The compositions, systems and methods provided herein may be used to selectively reduce the growth, reduce the viability, induce cell death or arrest the cell cycle of a cell, wherein the cell comprises one or more mutant KRAS allele.

### 7. Vectors

Compositions, systems, and methods described herein comprise a vector or a use thereof. A vector can comprise a nucleic acid of interest (e.g., a NRAS or KRAS-targeting guide nucleic acid or polynucleotide encoding the same). In some embodiments, the nucleic acid of interest comprises one or more components of a composition or system described herein (e.g., a NRAS or KRAS-targeting guide nucleic acid or polynucleotide encoding the same). In some embodiments, the nucleic acid of interest comprises a nucleotide sequence that encodes one or more components of the composition or system described herein. In some embodiments, one or more components comprises a polypeptide(s), guide nucleic acid(s), target nucleic acid(s), and donor nucleic acid(s). In some embodiments, the component comprises a nucleic acid encoding an effector protein and a guide nucleic acid or a nucleic acid encoding the guide nucleic acid. The vector may be part of a vector system, wherein a vector system comprises a library of vectors each encoding one or more component of a composition or system described herein. In some embodiments, components described herein (e.g., an effector protein, a guide nucleic acid, and/or a target nucleic acid) are encoded by the same vector. In some embodiments, components described herein (e.g., an effector protein, a guide nucleic acid, and/or a target nucleic acid) are each encoded by different vectors of the system.

In some embodiments, a vector comprises a nucleotide sequence encoding one or more effector proteins as described herein. In some embodiments, the one or more effector proteins comprise at least two effector proteins. In some embodiments, the at least two effector protein are the same. In some embodiments, the at least two effector proteins are different from each other. In some embodiments, the nucleotide sequence is operably linked to a promoter that is operable in a target cell, such as a eukaryotic cell. In some embodiments, the vector comprises the nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more effector proteins.

In some embodiments, a vector may encode one or more of any system components, including but not limited to effector proteins, guide nucleic acids, donor nucleic acids, and target nucleic acids as described herein. In some embodiments, a system component encoding sequence is operably linked to a promoter that is operable in a target cell, such as a eukaryotic cell. In some embodiments, a vector may encode 1, 2, 3, 4 or more of any system components. For example, a vector may encode two or more guide nucleic acids, wherein each guide nucleic acid comprises a different sequence. A vector may encode an effector protein and a guide nucleic acid. A vector may comprise the nucleic acid encoding an effector protein, a guide nucleic acid, and a donor nucleic acid.

In some embodiments, a vector comprises one or more guide nucleic acids, or a nucleotide sequence encoding the one or more guide nucleic acids as described herein (e.g., a NRAS or KRAS-targeting guide nucleic acid or polynucleotide encoding the same). In some embodiments, the one or more guide nucleic acids comprise at least two guide nucleic acids. In some embodiments, the at least two guide nucleic acids are the same. In some embodiments, the at least two guide nucleic acids are different from each other. In some embodiments, the guide nucleic acid or the nucleotide sequence encoding the guide nucleic acid is operably linked to a promoter that is operable in a target cell, such as a eukaryotic cell. In some embodiments, the vector comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids. In some embodiments, the vector comprises a nucleotide sequence encoding 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more guide nucleic acids.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human H1 promoter (H1). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of one or more genome editing tools described herein. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EF1a, RPBSA, hPGK, EFS, SV40, PGK1, Ubc, human beta actin, CAG, TRE, UAS, Ac5, Polyhedrin, CaMKIIa, GAL1-10, H1, TEF1, GDS, ADH1, HSV TK, Ubi, U6, MNDU3, MSCV, MND and CAG. In some embodiments, the promoter allows for expression in a melanocyte cell. Non-limiting examples of such promoters are TYR, MC1R, MIA, TERT, Cox-2, CXCR4, and BIRC5. In some embodiments, the promoter allows for expression in colon cells. In embodiments the promoter allows for expression in rectal cells. In some instance, the promoter allows for expression in thyroid follicular cells. In some embodiments, the promoter allows for expression in lymphocytes. In some embodiments, the promoter allows for expression in B lymphocytes. In some embodiments, the promoter allows for expression in Langerhans cells. Non-limiting examples of such promoters are Dectin-2. In some embodiments, the promoter allows for expression in epithelial lung cells.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing effector protein is a melanocyte-specific promoter. In some embodiments, the melanocyte-specific promoter comprises TYR, MC1R, MIA, TERT, Cox-2, CXCR4, and BIRC5 promoter sequence. In some embodiments, the promoter for expressing effector protein is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA.

In some embodiments, the expression vector comprises the DNA molecule encoding a guide nucleic acid. In some embodiments, the expression vector further comprises the nucleic acid encoding an effector protein. In some embodiments, the expression vector further comprises or encodes a donor nucleic acid. In some embodiments, the expression vector encoding a guide nucleic acid, wherein the guide nucleic acid comprises a first region comprising a repeat; and a second region comprising a spacer sequence that is complementary to a target sequence of a NRAS or KRAS target gene. In some embodiments, wherein the first region is located 5′ of the second region.

In some embodiments, the expression vector further comprises an effector protein that binds a repeat sequence or a nucleic acid encoding the effector protein. In some embodiments, the spacer comprises a nucleotide sequence that is at least 70%, at least 75%, at least 80%, at least 85%, at least 90%, at least 95%, or 100% identical to a sequence selected from SEQ ID NOs: 72-75; the repeat sequence comprises a nucleotide sequence that is at least 70%, at least 75%, at least 80%, at least 85%, at least 90%, at least 95%, or 100% identical to SEQ ID NOs: 81-88; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence selected from SEQ ID NOs: 1-2 and 24-43; or a combination thereof.

In some embodiments, the expression vector further comprises an effector protein that binds a repeat sequence or a nucleic acid encoding the effector protein. In some embodiments, the spacer comprises a nucleotide sequence that is at least 70%, at least 75%, at least 80%, at least 85%, at least 90%, at least 95%, or 100% identical to a sequence selected from SEQ ID NOs: 77, 78, and 80; the handle sequence comprises a nucleotide sequence that is at least 70%, at least 75%, at least 80%, at least 85%, at least 90%, at least 95%, or 100% identical to a sequence selected from SEQ ID NO: 90; the effector protein comprises an amino acid sequence that is at least 65%, at least 70%, at least 75%, at least 80%, at least 85%, at least 90%, at least 95%, at least 97%, at least 98%, at least 99%, or 100% identical to any sequence selected from SEQ ID NO: 3, 4 and 44-61; or a combination thereof.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo basepairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I.

In some embodiments, disclosed herein comprise one or more nucleic acids encoding an effector protein, fusion effector protein, fusion partner, a guide nucleic acid, or a combination thereof. The effector protein, fusion effector protein, fusion partner protein, or combination thereof may be any one of those described herein. In some embodiments, of the above, the nucleic acid expression vector comprises a polynucleotide encoding an effector protein that is at least 80%, at least 85%, at least 90%, at least 95%, at least 99%, or 100% identical to any one of the sequences recited in TABLE 1, TABLE 4 or TABLE 6.

The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein. In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

In some embodiments, a vector is administered as part of a method of nucleic acid detection, editing, and/or treatment as described herein. In some embodiments, a vector is administered in a single vehicle, such as a single expression vector. In some embodiments, at least two of the three components, a nucleic acid encoding one or more effector proteins, one or more donor nucleic acids, and one or more guide nucleic acids or a nucleic acid encoding the one or more guide nucleic acid, are provided in the single expression vector. In some embodiments, components, such as a guide nucleic acid and an effector protein, are encoded by the same vector. In some embodiments, an effector protein (or a nucleic acid encoding same) and/or a guide nucleic acid (or a nucleic acid that, when transcribed, produces same) are not co-administered with donor nucleic acid in a single vehicle. In some embodiments, an effector protein (or a nucleic acid encoding same), a guide nucleic acid (or a nucleic acid that, when transcribed, produces same), and/or donor nucleic acid are administered in one or more or two or more vehicles, such as one or more, or two or more expression vectors.

In some embodiments, a vector may be part of a vector system. In some embodiments, the vector system comprises a library of vectors each encoding one or more components of a composition or system described herein. In some embodiments, a vector system is administered as part of a method of nucleic acid detection, editing, and/or treatment as described herein, wherein at least two vectors are co-administered. In some embodiments, the at least two vectors comprise different components. In some embodiments, the at least two vectors comprise the same component having different sequences. In some embodiments, at least one of the three components, a nucleic acid encoding one or more effector proteins, one or more donor nucleic acids, and one or more guide nucleic acids or a nucleic acid encoding the one or more guide nucleic acids, or a variant thereof is provided in a different vector. In some embodiments, the nucleic acid encoding the effector protein, and a guide nucleic acid or a nucleic acid encoding the guide nucleic acid are provided in different vectors. In some embodiments, the donor nucleic acid is encoded by a different vector than the vector encoding the effector protein and the guide nucleic acid.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, compositions and methods comprise a lipid, polymer, nanoparticle, or a combination thereof, or use thereof, to introduce one or more effector proteins, one or more guide nucleic acids, one or more donor nucleic acids, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEG2000), derivatives, analogs, or variants thereof. In some embodiments, the LNP has a negative net overall charge prior to complexation with one or more of a guide nucleic acid, a nucleic acid encoding the one or more guide nucleic acid, a nucleic acid encoding the effector protein, and/or a donor nucleic acid. In some embodiments, the inner core is a hydrophobic core. In some embodiments, the one or more of a guide nucleic acid, the nucleic acid encoding the one or more guide nucleic acid, the nucleic acid encoding the effector protein, and/or the donor nucleic acid forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Delivery of Viral Vectors

In some embodiments, a vector described herein comprises a viral vector. In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector provided herein comprises a sequence encoding genome editing tools. In some embodiments, the genome editing tools comprise a nucleic acid encoding one or more effector proteins, a nucleic acid encoding one or more fusion proteins (e.g., a nuclear localization signal (NLS), polyA tail), one or more guide nucleic acids, a nucleic acid encoding the one or more guide nucleic acids, respective promoter(s), one or more donor nucleic acid, or any combinations thereof. In some embodiments, viral vectors provided herein comprise at least one promotor or a combination of promoters driving expression or transcription of one or more genome editing tools described herein. In some embodiments, a coding region of the AAV vector forms an intramolecular double-stranded DNA template thereby generating the AAV vector that is a self-complementary AAV (scAAV) vector. In some embodiments, the scAAV vector comprises the sequence encoding genome editing tools that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

In some embodiments, the AAV vector comprises a recombinant AAV expression cassette comprising sequences encoding: a) a first inverted terminal repeat (ITR) and a first promoter; b) an effector protein disclosed herein; c) optionally a second promoter; d) a second polynucleotide encoding a guide nucleic acid disclosed here; and e) a second ITR. In some embodiments, the AAV expression cassette is a self-complementary AAV vector.

### Producing AAV Delivery Vectors

In some embodiments, methods of producing AAV delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging an effector encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

In some embodiments, AAV particles described herein are recombinant AAV (rAAV). In some embodiments, rAAV particles are generated by transfecting AAV producing cells with an AAV-containing plasmid carrying the sequence encoding the genome editing tools, a plasmid that carries viral encoding regions, i.e., Rep and Cap gene regions; and a plasmid that provides the helper genes such as E1A, E1B, E2A, E4ORF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a variant thereof, or a combination thereof. In some embodiments, rAAV virus particles can be produced in the mammalian cell culture system by providing the rAAV plasmid to the mammalian cell. In some embodiments, producing rAAV virus particles in a mammalian cell comprises transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells may comprise baculovirus. In some embodiments, production of rAAV virus particles in insect cells may comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1; 13(16):1935-43; and Benskey et al., (2019), Methods Mol Biol., 1937:3-26, each of which is incorporated by reference in its entirety.

### 8. Pharmaceutical Compositions and Modes of Administration

Disclosed herein are compositions comprising one or more effector proteins described herein or nucleic acids encoding the one or more effector proteins, one or more guide nucleic acids described herein or nucleic acids encoding the one or more guide nucleic acids described herein (e.g., NRAS or KRAS-targeting guide nucleic acids or polynucleotides encoding the same), or combinations thereof. In some embodiments, a repeat sequence of the one or more guide nucleic acids are capable of interacting with the one or more of the effector proteins. In some embodiments, spacer sequences of the one or more guide nucleic acids hybridizes with a target sequence of a target nucleic acid. In some embodiments, the compositions are capable of editing a target nucleic acid in a cell or a subject. In some embodiments, the compositions are capable of editing a target nucleic acid or the expression thereof in a cell, in a tissue, in an organ, in vitro, in vivo, or ex vivo. In some embodiments, the compositions are capable of editing a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

In some embodiments, compositions described herein are pharmaceutical compositions. In some embodiments, the pharmaceutical compositions comprise compositions described herein and a pharmaceutically acceptable carrier or diluent. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethylene glycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²⁺SO₄²⁻.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

Disclosed herein, in some embodiments, are pharmaceutical compositions for modifying a target nucleic acid in a cell or a subject, comprising any one of the effector proteins, engineered effector proteins, fusion effector proteins, or guide nucleic acids as described herein and any combination thereof. Also disclosed herein, in some embodiments, are pharmaceutical compositions comprising a nucleic acid encoding any one of the effector proteins, engineered effector proteins, fusion effector proteins, or guide nucleic acids as described herein and any combination thereof. Also disclosed herein, are pharmaceutical compositions comprising the nucleic acid expression vector, the cell, or the population of cells disclosed herein. In some embodiments, pharmaceutical compositions comprise a plurality of guide nucleic acids. In some embodiments, the pharmaceutical composition disclosed herein also comprise a pharmaceutical acceptable carrier. Pharmaceutical compositions may be used to modify a target nucleic acid or the expression thereof in a cell in vitro, in vivo or ex vivo. In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein.

### 9. Methods of Nucleic Acid Modification

Provided herein are methods of editing and modifying the expression of target nucleic acids (e.g., a target nucleic acid in the NRAS or KRAS gene or a mutated version thereof). In general, editing refers to modifying the nucleobase sequence of a target nucleic acid. However, compositions and systems disclosed herein may also be capable of making epigenetic modifications of target nucleic acids. Effector proteins, multimeric complexes thereof and systems described herein may be used for editing or modifying a target nucleic acid. Editing a target nucleic acid may comprise one or more of: cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

Methods of editing may comprise contacting a target nucleic acid with an effector protein described herein and a guide nucleic acid, wherein the effector protein comprises an amino acid sequence that is at least 70%, at least 75%, at least 80%, at least 85%, at least 90%, at least 95%, at least 97%, or at least 98%, at least 99%, or 100% identical to any one of the sequences set forth in TABLE 1, TABLE 4 or TABLE 6. In some embodiments, embodiments, the effector protein comprises an amino acid substitution relative to SEQ ID NO: 1 selected from the group consisting of L26R, E109R, H208R, K184R, K38R, L182R, Q183R, S108R, S198R, and T114R. In some embodiments, the effector protein is a dCas protein. In some embodiments, the dCas protein comprises an amino acid substation of D369A, D369N, D658A, D658N, E567A, and E567Q relative to SEQ ID NO: 1. In some embodiments, embodiments, the effector protein comprises an amino acid substitution relative to SEQ ID NO: 3 selected from the group consisting of D220R, N286K, E225K, I80K, S209F, Y315M, N193K, M298L, M295W, A306K, A218K, and K58W. In some embodiments, the effector protein is a dCas protein. In some embodiments, the dCas protein comprises an amino acid substation of E335Q, D237A D418A, D418N, and E335A relative to SEQ ID NO: 3.

In some embodiments, the guide nucleic acid comprises a spacer sequence that is at least 70%, at least 75%, at least 80%, at least 85%, at least 90%, at least 92%, at least 95%, or 100% identical to any one of SEQ ID NOs: 72-75 and a repeat sequence that is at least 70%, at least 75%, at least 80%, at least 85%, at least 90%, at least 92%, at least 95%, or 100% identical to SEQ ID NOs: 81-88.

In some embodiments, the guide nucleic acid comprises a spacer sequence that is at least 70%, at least 75%, at least 80%, at least 85%, at least 90%, at least 92%, at least 95%, or 100% identical to any one of SEQ ID NOs: 77, 78 and 80 and a handle sequence that is at least 70%, at least 75%, at least 80%, at least 85%, at least 90%, at least 92%, at least 95%, or 100% identical to any one of SEQ ID NO: 90.

Editing may introduce a mutation (e.g., point mutations, deletions) in a target nucleic acid relative to a corresponding wildtype nucleobase sequence. Editing may remove or correct a disease-causing mutation in a nucleic acid sequence to produce a corresponding wildtype nucleobase sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Editing may comprise single stranded cleavage, double stranded cleavage, donor nucleic acid insertion, epigenetic modification (e.g., methylation, demethylation, acetylation, or deacetylation), or a combination thereof. In some embodiments, cleavage (single-stranded or double-stranded) is site-specific, meaning cleavage occurs at a specific site in the target nucleic acid, often within the region of the target nucleic acid that hybridizes with the guide nucleic acid spacer region. In some embodiments, the target nucleic acid, and the resulting cleaved nucleic acid is contacted with a nucleic acid for homologous recombination (e.g., homology directed repair (HDR)) or non-homologous end joining (NHEJ). In some cases, a double-stranded break in the target nucleic acid may be repaired (e.g., by NHEJ or HDR) without insertion of a donor template, such that the repair results in an indel in the target nucleic acid at or near the site of the double-stranded break.

In some embodiments, an indel, sometimes referred to as an insertion-deletion or indel mutation, is a type of genetic mutation that results from the insertion and/or deletion of nucleotides in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it is also a frameshift mutation.

In some embodiments, wherein the compositions, systems, and methods of the present disclosure comprise an additional guide nucleic acid or a use thereof, the dual-guided compositions, systems, and methods described herein can modify the target nucleic acid in two locations. In some cases, dual-guided editing can comprise cleavage of the target nucleic acid in the two locations targeted by the guide RNAs. In certain embodiments, upon removal of the sequence between the guide nucleic acids, the wild-type reading frame is restored. A wild-type reading frame can be a reading frame that produces at least a partially, or fully, functional protein. A non-wild-type reading frame can be a reading frame that produces a non-functional or partially non-functional protein.

Accordingly, in some embodiments, compositions, systems, and methods described herein can edit 1 to 1,000 nucleotides or any integer in between, in a target nucleic acid. In certain embodiments, 1 to 1,000, 2 to 900, 3 to 800, 4 to 700, 5 to 600, 6 to 500, 7 to 400, 8 to 300, 9 to 200, or 10 to 100 nucleotides, or any integer in between, can be edited by the compositions, systems, and methods described herein. In some embodiments, 1, 2, 3, 4, 5, 6, 7, 8, 9, 10, or more nucleotides can be edited by the compositions, systems, and methods described herein. In some embodiments, 10, 20, 30, 40, 50, 60, 70, 80 90, 100 or more nucleotides, or any integer in between, can be edited by the compositions, systems, and methods described herein. In some embodiments, 100, 200, 300, 400, 500, 600, 700, 800, 900 or more nucleotides, or any integer in between, can be edited by the compositions, systems, and methods described herein.

In some cases, methods comprise editing a target nucleic acid with two or more effector proteins. Editing a target nucleic acid may comprise introducing a two or more single-stranded breaks in a target nucleic acid. In some embodiments, a break may be introduced by contacting a target nucleic acid with an effector protein and a guide nucleic acid. The guide nucleic acid may bind to the effector protein and hybridize to a region of the target nucleic acid, thereby recruiting the effector protein to the region of the target nucleic acid. Binding of the effector protein to the guide nucleic acid and the region of the target nucleic acid may activate the effector protein, and the effector protein may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first effector protein and hybridizes to a first region of the target nucleic acid and a second guide nucleic acid that binds to a second programmable nickase and hybridizes to a second region of the target nucleic acid. The first effector protein may introduce a first break in a first strand at the first region of the target nucleic acid, and the second effector protein may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with donor nucleic acid), thereby modifying the target nucleic acid.

Methods, systems and compositions described herein can edit or modify a target nucleic acid wherein such editing or modification can be measured by indel activity. Indel activity measures the amount of change in a target nucleic acid (e.g., nucleotide deletion(s) and/or insertion(s)) compared to a target nucleic acid that has not been contacted by a polypeptide described in compositions, systems, and methods described herein. For example, indel activity can be detected by next generation sequencing of one or more target loci of a target nucleic acid where indel percentage is calculated as the fraction of sequencing reads containing insertions or deletions relative to an unedited reference sequence. In certain embodiments, methods, systems, and compositions comprising an effector protein and guide nucleic acid described herein can exhibit about 0.0001% to about 65% or more indel activity upon contact to a target nucleic acid compared to a target nucleic acid non-contacted with compositions, systems, or by methods described herein. For example, methods, systems, and compositions comprising an effector protein and guide nucleic acid described herein can exhibit about 0.0001%, about 0.001%, about 0.01%, about 0.1%, about 1%, about 5%, about 10%, about 15%, about 20%, about 25%, about 30%, about 35%, about 40%, about 45%, about 50%, about 55%, about 60%, about 65% or more indel activity.

In certain embodiments, sequence deletion is a modification where one or more sequences in a target nucleic acid are deleted relative to a target nucleic acid without the sequence deletion. In certain embodiments, a sequence deletion can result in or effect a splicing disruption or a frameshift mutation. In certain embodiments, a sequence deletion result in or effect a splicing disruption.

In certain embodiments, a modification is a deletion of an entire exon. In some embodiments, the exon is associated with a disease. In some embodiments, the guide nucleic acids disclosed herein target a A182T mutation of the NRAS gene.

In certain embodiments, a modification is a deletion of an entire exon. In some embodiments, the exon is associated with a disease. In some embodiments, the guide nucleic acids disclosed herein target a G35A mutation of the KRAS gene.

In some embodiments, compositions, systems, and methods described herein comprise a combination of a first gRNA, a second gRNA, a first effector protein, and a second effector protein, wherein the combination can be used for deleting the entire exon or a portion thereof. In some embodiments, the first effector protein and the second effector protein are the same. In some embodiments, the first effector protein and the second effector protein are not the same.

In certain embodiments, sequence skipping is a modification where one or more sequences in a target nucleic acid are skipped upon transcription or translation of the target nucleic acid relative to a target nucleic acid without the sequence skipping. In certain embodiments, sequence skipping can result in or effect a splicing disruption or a frameshift mutation. In certain embodiments, sequence skipping can result in or effect a splicing disruption.

In certain embodiments, sequence reframing is a modification where one or more bases in a target are modified so that the reading frame of the sequence is reframed relative to a target nucleic acid without the sequence reframing. In certain embodiments, sequence reframing can result in or effect a splicing disruption or a frameshift mutation. In certain embodiments, sequence reframing can result in or effect a frameshift mutation.

In certain embodiments, sequence knock-in is a modification where one or more sequences is inserted into a target nucleic acid relative to a target nucleic acid without the sequence knock-in. In certain embodiments, sequence knock-in can result in or effect a splicing disruption or a frameshift mutation. In certain embodiments, sequence knock-in can result in or effect a splicing disruption.

In certain embodiments, editing or modification of a target nucleic acid can be locus specific, wherein compositions, systems, and methods described herein can edit or modify a target nucleic acid at one or more specific loci to effect one or more specific mutations comprising splicing disruption mutations, frameshift mutations, sequence deletion, sequence skipping, sequence reframing, sequence knock-in, or any combination thereof. For example, editing or modification of a specific locus can affect any one of a splicing disruption, frameshift (e.g., 1+ or 2+ frameshift), sequence deletion, sequence skipping, sequence reframing, sequence knock-in, or any combination thereof. In certain embodiments, editing or modification of a target nucleic acid can be locus specific, modification specific, or both. In certain embodiments, editing or modification of a target nucleic acid can be locus specific, modification specific, or both, wherein compositions, systems, and methods described herein comprise an effector protein described herein and a guide nucleic acid described herein.

Methods of editing a target nucleic acid or modulating the expression of a target nucleic acid may be performed in vivo. Methods of editing a target nucleic acid or modulating the expression of a target nucleic acid may be performed in vitro. Methods of editing a target nucleic acid or modulating the expression of a target nucleic acid may be performed ex vivo. Editing methods include, but are not limited to, introduction of double stranded breaks (DSB), which can result in deleting some nucleotides and disrupting the translation of a functional protein, base editing, and splice acceptor disruption (SA).

In some embodiments, the method of editing by the effector proteins can be promotor silencing, frameshift mutation, base editing, or splice disruption.

In some embodiments, the editing by the effector protein targets an exon of the NRAS or KRAS gene. In some embodiments, the editing by the effector protein targets an intron of the NRAS or KRAS gene. In some embodiments, the editing by the effector protein targets a SNP of the NRAS or KRAS gene. In some embodiments, the editing by the effector protein targets the 3′ UTR of the NRAS or KRAS gene. In some embodiments, the editing by the effector protein targets the poly-A tail of the NRAS or KRAS gene. In some embodiments, the editing by the effector protein decreases transcription of the DNA sequence of the NRAS or KRAS gene. In some embodiments, the editing by the effector protein decreases translation of the RNA sequence of the NRAS or KRAS gene.

In some embodiments, the gene regulation is regulated by effector protein repressing a promoter. In some embodiments the repression is temporary or transient. In some embodiments the repression is permanent. In some embodiments the effector protein is fused to a KRAB sequence. In some embodiments the effector protein is fused to an acetylase sequence. In some embodiments the effector protein is fused to a methyltransferase. In some embodiments the effector protein is fused to a Ezh2 sequence.

In some embodiments, the effector protein causes a frameshift mutation. In some embodiments the effector protein causes the addition of one or more nucleotides causing a shift in the reading frame. In some embodiments the effector protein causes a deletion of one or more nucleotides causing a shift in the reading frame. In some embodiments the effector protein causes the deletion or addition of 1, 2, or 4 nucleotides. In some embodiments the effector protein causes an alternation in the amino acid sequence at protein translation. In some embodiments the alteration is a missense mutation. In some embodiments the alteration is a premature stop codon. In some embodiments the effector protein causes a change in the ribosome reading frame and cause premature termination of translation at a new nonsense or chain termination codon (TAA, TAG, and TGA).

In some embodiments the effector protein causes a nucleobase to be edited. In some embodiments the effector protein is fused to an adenine base editing enzyme (ABE). In some embodiments the effector protein is fused to a cytosine base editing enzyme (CBE). In some embodiments the fusion protein causes a cytosine to thymidine transition. In some embodiments the fusion protein causes a cytosine to uracil transition. In some embodiments the fusion protein causes a thymidine to cytosine transition. In some embodiments the fusion protein causes an adenosine to guanosine transition. In some embodiments the fusion protein causes a guanosine to adenosine conversion. In some embodiments the alteration results in a missense mutation. In some embodiments the alteration is a premature stop codon. In some embodiments the fusion protein causes a premature termination of translation at a new nonsense or chain termination codon (TAA, TAG, and TGA).

### 10. Methods of Detecting a Target Nucleic Acid

Provided herein are methods of detecting target nucleic acids. Methods may comprise detecting target nucleic acids with compositions or systems described herein. Methods may comprise detecting a target nucleic acid in a sample, e.g., a cell lysate, a biological fluid, or environmental sample. Methods may comprise detecting a target nucleic acid in a cell. In some embodiments, methods of detecting a target nucleic acid in a sample or cell comprises contacting the sample or cell with an effector protein or a multimeric complex thereof, a guide nucleic acid, wherein at least a portion of the guide nucleic acid is complementary to at least a portion of the target nucleic acid, and a reporter nucleic acid that is cleaved in the presence of the effector protein, the guide nucleic acid, and the target nucleic acid, and detecting a signal produced by cleavage of the reporter nucleic acid, thereby detecting the target nucleic acid in the sample. In some embodiments, methods result in trans cleavage of the reporter nucleic acid. In some embodiments, methods result in cis cleavage of the reporter nucleic acid.

### 11. Method of Treating a Disorder

Described herein are methods for treating or preventing a disease in a subject by modifying a target nucleic acid associated with a gene or expression of a gene related to the disease. In some embodiments, the disease or disorder comprises an increase in NRAS or KRAS expression. In some embodiments, the disease or disorder is a cancer. In some embodiments, the cancer is melanoma. In some embodiments the melanoma is a cutaneous melanoma. In some embodiments, the melanoma is metastatic. In some embodiments, the cancer is a colon adenocarcinoma. In some embodiments, the cancer is a colorectal carcinoma. In some embodiments, the cancer is a hairy cell leukemia. In some embodiments, the cancer is a Langerhans cell histiocytosis. In some embodiments, the cancer is non-Hodgkin lymphoma. In some embodiments, the cancer is a thyroid gland papillary carcinoma. In some embodiments, the cancer is a non-small cell lung carcinoma. In some embodiments, the cancer is a lung adenocarcinoma.

In some embodiments, the present disclosure provides methods of treating a cancer in a subject in need thereof comprising administering a system described herein comprising a guide RNA targeting the NRAS gene or a mutated version thereof. In some embodiments, the mutated version of the NRAS gene comprises a A182T mutation encoding the NRAS Q61L mutated protein. In some embodiments, the present disclosure provides methods of killing a cancer cell comprising administering a system described herein comprising a guide RNA targeting the NRAS gene or a mutated version thereof. In some embodiments, the present disclosure provides methods of killing a cancer cell comprising administering a system described herein comprising an effector protein and a guide RNA that specifically modify a NRAS mutant allele. In some embodiments, the cancer cell is in vitro. In some embodiments, the cancer cell is in vivo.

In some embodiments, the present disclosure provides methods of treating a cancer in a subject in need thereof comprising administering a system described herein comprising a guide RNA targeting the KRAS gene or a mutated version thereof. In some embodiments, the mutated version of the KRAS gene comprises a mutation encoding the KRAS G12D mutated protein. In some embodiments, the present disclosure provides methods of killing a cancer cell comprising administering a system described herein comprising a guide RNA targeting the KRAS gene or a mutated version thereof. In some embodiments, the present disclosure provides methods of killing a cancer cell comprising administering a system described herein comprising an effector protein and a guide RNA that specifically modify a KRAS mutant allele. In some embodiments, the cancer cell is in vitro. In some embodiments, the cancer cell is in vivo.

In some embodiments, methods comprise administering a guide RNA comprising one or more sequences selected from the sequences in TABLES 10-13, and SEQ ID NOs: 90-92 or a nucleic acid encoding the same. In some embodiments, methods comprise administering a Cas protein or a nucleic acid encoding the same. In some embodiments, the Cas protein comprises an amino acid sequence that is at least 90% or 95% identical to any one of the sequences recited in TABLE 1, TABLE 4 or TABLE 6. The Cas protein or nucleic acid encoding the same, and the guide RNA or nucleic acid encoding the same may be administered in a single composition. The Cas protein or nucleic acid encoding the same, and the guide RNA or nucleic acid encoding the same may be administered separately (formulaically or chronologically). In some embodiments, methods comprise administering: a Cas protein or a messenger RNA encoding a Cas protein and a lipid nanoparticle; and a viral vector encoding a guide RNA. In some embodiments, methods comprise administering a viral vector encoding the Cas protein and the guide RNA. In some embodiments, methods comprise administering a Cas protein and a lipid nanoparticle. In some embodiments, methods comprise administering a messenger RNA encoding a Cas protein.

### Sequences and Tables

TABLE 1
Exemplary Effector Proteins

| Effector | | SEQ ID |
|---|---|---|
| Protein | Amino acid sequence | NO: |
| CasPhi.12 | MIKPTVSQFLTPGFKLIRNHSRTAGLKLKNEGEEACKKFVREN | 1 |
| | EIPKDECPNFQGGPAIANIIAKSREFTEWEIYQSSLAIQEVIFTLP | |
| | KDKLPEPILKEEWRAQWLSEHGLDTVPYKEAAGLNLIIKNAVN | |
| | TYKGVQVKVDNKNKNNLAKINRKNEIAKLNGEQEISFEEIKAF | |
| | DDKGYLLQKPSPNKSIYCYQSVSPKPFITSKYHNVNLPEEYIGY | |
| | YRKSNEPIVSPYQFDRLRIPIGEPGYVPKWQYTFLSKKENKRRK | |
| | LSKRIKNVSPILGIICIKKDWCVFDMRGLLRTNHWKKYHKPTD | |
| | SINDLFDYFTGDPVIDTKANVVRFRYKMENGIVNYKPVREKKG | |
| | KELLENICDQNGSCKLATVDVGQNNPVAIGLFELKKVNGELTK | |
| | TLISRHPTPIDFCNKITAYRERYDKLESSIKLDAIKQLTSEQKIEV | |
| | DNYNNNFTPQNTKQIVCSKLNINPNDLPWDKMISGTHFISEKA | |
| | QVSNKSEIYFTSTDKGKTKDVMKSDYKWFQDYKPKLSKEVRD | |
| | ALSDIEWRLRRESLEFNKLSKSREQDARQLANWISSMCDVIGIE | |
| | NLVKKNNFFGGSGKREPGWDNFYKPKKENRWWINAIHKALT | |
| | ELSQNKGKRVILLPAMRTSITCPKCKYCDSKNRNGEKFNCLKC | |
| | GIELNADIDVATENLATVAITAQSMPKPTCERSGDAKKPVRAR | |
| | KAKAPEFHDKLAPSYTVVLREAV | |
| CasPhi.12 | PKKKRKVGIHGVPAAMIKPTVSQFLTPGFKLIRNHSRTAGLKLK | 2 |
| with NLS | NEGEEACKKFVRENEIPKDECPNFQGGPAIANIIAKSREFTEWEI | |
| | YQSSLAIQEVIFTLPKDKLPEPILKEEWRAQWLSEHGLDTVPYK | |
| | EAAGLNLIIKNAVNTYKGVQVKVDNKNKNNLAKINRKNEIAK | |
| | LNGEQEISFEEIKAFDDKGYLLQKPSPNKSIYCYQSVSPKPFITS | |
| | KYHNVNLPEEYIGYYRKSNEPIVSPYQFDRLRIPIGEPGYVPKW | |
| | QYTFLSKKENKRRKLSKRIKNVSPILGIICIKKDWCVFDMRGLL | |
| | RTNHWKKYHKPTDSINDLFDYFTGDPVIDTKANVVRFRYKME | |
| | NGIVNYKPVREKKGKELLENICDQNGSCKLATVDVGQNNPVA | |
| | IGLFELKKVNGELTKTLISRHPTPIDFCNKITAYRERYDKLESSI | |
| | KLDAIKQLTSEQKIEVDNYNNNFTPQNTKQIVCSKLNINPNDLP | |
| | WDKMISGTHFISEKAQVSNKSEIYFTSTDKGKTKDVMKSDYK | |
| | WFQDYKPKLSKEVRDALSDIEWRLRRESLEFNKLSKSREQDAR | |
| | QLANWISSMCDVIGIENLVKKNNFFGGSGKREPGWDNFYKPK | |
| | KENRWWINAIHKALTELSQNKGKRVILLPAMRTSITCPKCKYC | |
| | DSKNRNGEKFNCLKCGIELNADIDVATENLATVAITAQSMPKP | |
| | TCERSGDAKKPVRARKAKAPEFHDKLAPSYTVVLREAVKRPA | |
| | ATKKAGQAKKKK | |
| CasM. | MSVLTRKVQLIPVGDKEERDRVYKYLRDGIEAQNRAMNLYM | 3 |
| 265466 | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKIIL | |
| | NMAMDIPDKEIELDEDVCVGVDLGIAIPAVCALNKNRYSRVSI | |
| | GSKEDFLRVRTKIRNQRKRLQTNLKSSNGGHGRKKKMKPMD | |
| | RFRDYEANWVQNYNHYVSRQVVDFAVKNKAKYINLENLEGI | |
| | RDDVKNEWLLSNWSYYQLQQYITYKAKTYGIEVRKINPYHTS | |
| | QRCSCCGYEDAGNRPKKEKGQAYFKCLKCGEEMNADFNAAR | |
| | NIAMSTEFQSGKKTKKQKKEQHENK | |
| 3x Flag- | MDYKDHDGDYKDHDIDYKDDDDKMAPKKKRKVGIHGVPAAM | 4 |
| SV40NLS- | SVLTRKVQLIPVGDKEERDRVYKYLRDGIEAQNRAMNLYMSG | |
| CasM.265466- | LYFAAINEASKEDRKELNQLYSRIATSSKGSAYTTDIEFPTGLA | |
| nucleoplasmin | STSTLSMAVRQDFTKSLKDGLMYGRVSLPTYRKDNPLFVDVR | |
| NLS | FVALRGTKQKYNGLYHEYKSHTEFLDNLYSSDLKVYIKFANDI | |
| | TFQVIFGNPRKSSALRSEFQNIFEEYYKVCQSSIQFSGTKIILNM | |
| | AMDIPDKEIELDEDVCVGVDLGIAIPAVCALNKNRYSRVSIGSK | |
| | EDFLRVRTKIRNQRKRLQTNLKSSNGGHGRKKKMKPMDRFRD | |
| | YEANWVQNYNHYVSRQVVDFAVKNKAKYINLENLEGIRDDV | |
| | KNEWLLSNWSYYQLQQYITYKAKTYGIEVRKINPYHTSQRCS | |
| | CCGYEDAGNRPKKEKGQAYFKCLKCGEEMNADFNAARNIAM | |
| | STEFQSGKKTKKQKKEQHENKKRPAATKKAGQAKKKK | |

TABLE 2
Exemplary Nuclear Localization Sequences

| SEQ ID NO: | Description | SEQUENCES |
|---|---|---|
| 5 | NLS | PKKKRKVGIHGVPAA |
| 6 | NLS | KRPAATKKAGQAKKKK |
| 7 | NLS | KR(K/R)R |
| 8 | NLS | (P/R)XXKR(\hat{ }DE)(K/R) |
| 9 | NLS | KRX(W/F/Y)XXAF |
| 10 | NLS | (R/P)XXKR(K/R)(\hat{ }DE) |
| 11 | NLS | LGKR(K/R)(W/F/Y) |
| 12 | NLS | KRX₁₀K(K/R)(K/R) |
| 13 | NLS | K(K/R)RK |
| 14 | NLS | KRX₁₁K(K/R)(K/R) |
| 15 | NLS | KRX₁₂K(K/R)(K/R) |
| 16 | NLS | KRX₁₀K(K/R)X(K/R) |
| 17 | NLS | KRX₁₁K(K/R)X(K/R) |
| 18 | NLS | KRX₁₂K(K/R)X(K/R) |
| 19 | NLS | APKKKRKVGIHGVPAA |
| 20 | EEP | GLFXALLXLLXSLWXLLLXA |
| 21 | EEP | GLFHALLHLLHSLWHLLLHA |

X is any naturally occurring amino acid; and \hat{ }D/E is any naturally occurring amino acid except Asp or Glu

TABLE 3
Exemplary Amino Acid Alterations Relative to CasPhi.12 of SEQ ID NO: 1

| Effects | Amino Acid Alterations |
|---|---|
| | At least one substitution (i.e., with R, K or H) selected from I2, T5, K15, R18, |
| | H20, S21, L26, N30, E33, E34, A35, K37, K38, R41, N43, Q54, Q79R, |
| | K92E, K99R, S108, E109, H110, G111, D113, T114, P116, K118, E119, |
| | A121, N132, K135, Q138, V139, N148, L149, E157, E164, E166, E170, |
| | Y180, L182, Q183, K184, S186, K189, S196, S198, K200, I203, S205, K206, |
| | Y207, H208, N209, Y220, S223, E258, K281, K348, N355, S362, N406, |
| | K435, I471, I489, Y490, F491, D495, K496, K498, K500, D501, V502, |
| | K504, S505, D506, V521, E567, N568, S579, Q612, S638, F701, and P707 |
| Enhanced | T5R, L26R, L26K, A121Q, N148R, V139R, S198R, H208R, S223P, E258K, |
| nuclease | N355R, I471T, S579R, F701R, P707R, K189P, S638K, Q54R, Q79R, |
| activity relative | Y220S, N406K, E119S, K92E, K435Q, N568D, and V521T |
| to the wild-type | Double mutations: L26K/A121Q, L26X/A121Q, K99R/L149R, |
| effector protein | K99R/N148R, L149R/H208R, S362R/L26X L26X/N148R, L26X/H208R, |
| | N30R/N148R, L26X/K99R, L26X/P707R, L26X/L149R, L26X/N30R, |
| | L26X/N355R, L26X/K281R, L26X/S108R, L26X/K348R, T5R/V139R, |
| | I2R/V139R, K99R/S186R, L26X/A673G, L26X/Q674R, S579R/L26K, |
| | F701R/E258K, T5R/L26K, L26X/K435Q, L26X/G685R, L26X/Q674K, |
| | L26X/P699R, L26X/T70E, L26X/Q232R, L26X/T252R, L26X/E567Q, |
| | L26X/P679R, L26X/E83K, L26X/E73P, L26X/K248E, L26X, T5R/ S223P, |
| | S579R/ S223P, L26X/ S223P, T5R/ A121Q, L26X/ A696R, S198R/ I471T, |
| | L26X/ N153R, L26X/ E682R, L26X/ D703R, Q612R/ L26K, L26X/ I471T, |
| | K348R/ L26K, S579R/I471T, L26X/V228R, T5R/S638K, S579R/K189P, |
| | S579R/E258K, L26X/K260R, L26X/S638K, S579R/Y220S, T5R/I471T, |
| | L26X/F233R, L26X/V521T, F701R/A121Q, L26X/G361R, S198R/E258K, |
| | L26X/S472R, T5R/Y220S, L26X/A150K, L26X/S684R, L26X/E157R, |
| | L26X/K248R, F701R/L26K, S198R/N406K, S198R/Y220S, S198R/S638K, |
| | S198R/V521T, S579R/A121Q, K348R/Y220S, S198R/K189P, |
| | L26X/E242R, L26X/K678R, T5R/N406K, L26X/I158K, T5R/V521T, |
| | L26X/N259R, L26X/K257R, L26X/K256R, T5R/K189P, L26X/C405R, |
| | S579R/V521T, S579R/N406K, T5R/K92E, T5R/E258K, L26X/I97R, |
| | S579R/S638K, T5R/K435Q, F701R/S638K, L26X/L236R, F701R/I471T, |
| | Q612R/S223P, F701R/S223P, S198R/E119S, S579R/K92E, L26X/E715R, |
| | Q612R/1471T, F701R/Y220S, S198R/S223P, and L26X/K266R, wherein X |
| | is selected from R and K. |
| Nickase | E157A, E164A, E164L, E166A, E166I, E170A, I489A, I489S, Y490S, |
| activity | Y490A, F491A, F491S, F491G, D495G, D495R, D495K, K496A, K496S, |
| | K498A, K498S, K500A, K500S, D501R, D501G, D501K, V502A, V502S, |
| | K504A, K504S, S505R, D506A; |
| | deletion of S478-S505 of SEQ ID NO: 1; |
| | deletion of S478-S505 of SEQ ID NO: 1 and insertion of the sequence of |
| | SDLYIERGGDPRDVHQQVETKPKGKRKSEIRILKIR (SEQ ID NO: |
| | 22); |
| | deletion of S478-S505 of SEQ ID NO: 1 and insertion of the sequence of |
| | SDYIVDHGGDPEKVFFETKSKKDKTKRYKRR (SEQ ID NO: 23); |
| | an amino acid sequence that is at least 90%, at least 95%, at least 97%, at |
| | least 98%, at least 99% identical, or is 100% identical to; |
| | an amino acid sequence that is at least 90%, at least 95%, at least 97%, at |
| | least 98%, at least 99% identical, or is 100% identical to |
| Reduced or | D369A, D369N, D658A, D658N, E567A, E567Q |
| abolished | |
| nuclease | |
| activity relative | |
| to the wild-type | |
| effector protein | |

TABLE 4
Exemplary Engineered CasPhi.12 Effector Protein Variants

| | | SEQ |
|---|---|---|
| | | ID |
| Effector Protein | Amino Acid Sequence | NO: |
| CasPhi.12 L26R | MIKPTVSQFLTPGFKLIRNHSRTAGRKLKNEGEEACKK | 24 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| 3x Flag-SV40NLS- | MDYKDHDGDYKDHDIDYKDDDDKMAPKKKRKVGIH | 25 |
| CasPhi12 L26R- | GVPAAMIKPTVSQFLTPGFKLIRNHSRTAGRKLKNEGE | |
| NLS | EACKKFVRENEIPKDECPNFQGGPAIANIIAKSREFTEW | |
| | EIYQSSLAIQEVIFTLPKDKLPEPILKEEWRAQWLSEHG | |
| | LDTVPYKEAAGLNLIIKNAVNTYKGVQVKVDNKNKN | |
| | NLAKINRKNEIAKLNGEQEISFEEIKAFDDKGYLLQKPS | |
| | PNKSIYCYQSVSPKPFITSKYHNVNLPEEYIGYYRKSNE | |
| | PIVSPYQFDRLRIPIGEPGYVPKWQYTFLSKKENKRRKL | |
| | SKRIKNVSPILGIICIKKDWCVFDMRGLLRTNHWKKYH | |
| | KPTDSINDLFDYFTGDPVIDTKANVVRFRYKMENGIVN | |
| | YKPVREKKGKELLENICDQNGSCKLATVDVGQNNPVA | |
| | IGLFELKKVNGELTKTLISRHPTPIDFCNKITAYRERYD | |
| | KLESSIKLDAIKQLTSEQKIEVDNYNNNFTPQNTKQIVC | |
| | SKLNINPNDLPWDKMISGTHFISEKAQVSNKSEIYFTST | |
| | DKGKTKDVMKSDYKWFQDYKPKLSKEVRDALSDIEW | |
| | RLRRESLEFNKLSKSREQDARQLANWISSMCDVIGIEN | |
| | LVKKNNFFGGSGKREPGWDNFYKPKKENRWWINAIH | |
| | KALTELSQNKGKRVILLPAMRTSITCPKCKYCDSKNRN | |
| | GEKFNCLKCGIELNADIDVATENLATVAITAQSMPKPT | |
| | CERSGDAKKPVRARKAKAPEFHDKLAPSYTVVLREAV | |
| | KGRRPRKRPARQKRKRNS | |
| CasPhi.12 E567A | MIKPTVSQFLTPGFKLIRNHSRTAGLKLKNEGEEACKK | 26 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIANLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 E567Q | MIKPTVSQFLTPGFKLIRNHSRTAGLKLKNEGEEACKK | 27 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIQNLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 E109R | MIKPTVSQFLTPGFKLIRNHSRTAGLKLKNEGEEACKK | 28 |
| | FVRENEIPKDECPNFQGGPAIANIIAKSREFTEWEIYQSS | |
| | LAIQEVIFTLPKDKLPEPILKEEWRAQWLSRHGLDTVP | |
| | YKEAAGLNLIIKNAVNTYKGVQVKVDNKNKNNLAKIN | |
| | RKNEIAKLNGEQEISFEEIKAFDDKGYLLQKPSPNKSIY | |
| | CYQSVSPKPFITSKYHNVNLPEEYIGYYRKSNEPIVSPY | |
| | QFDRLRIPIGEPGYVPKWQYTFLSKKENKRRKLSKRIK | |
| | NVSPILGIICIKKDWCVFDMRGLLRTNHWKKYHKPTDS | |
| | INDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 H208R | MIKPTVSQFLTPGFKLIRNHSRTAGLKLKNEGEEACKK | 29 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYRNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 K184R | MIKPTVSQFLTPGFKLIRNHSRTAGLKLKNEGEEACKK | 30 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQRPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 K38R | MIKPTVSQFLTPGFKLIRNHSRTAGLKLKNEGEEACKR | 31 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 L182R | MIKPTVSQFLTPGFKLIRNHSRTAGLKLKNEGEEACKK | 32 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLRQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 Q183R | MIKPTVSQFLTPGFKLIRNHSRTAGLKLKNEGEEACKK | 33 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLRKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 S108R | MIKPTVSQFLTPGFKLIRNHSRTAGLKLKNEGEEACKK | 34 |
| | FVRENEIPKDECPNFQGGPAIANIIAKSREFTEWEIYQSS | |
| | LAIQEVIFTLPKDKLPEPILKEEWRAQWLREHGLDTVP | |
| | YKEAAGLNLIIKNAVNTYKGVQVKVDNKNKNNLAKIN | |
| | RKNEIAKLNGEQEISFEEIKAFDDKGYLLQKPSPNKSIY | |
| | CYQSVSPKPFITSKYHNVNLPEEYIGYYRKSNEPIVSPY | |
| | QFDRLRIPIGEPGYVPKWQYTFLSKKENKRRKLSKRIK | |
| | NVSPILGIICIKKDWCVFDMRGLLRTNHWKKYHKPTDS | |
| | INDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 S198R | MIKPTVSQFLTPGFKLIRNHSRTAGLKLKNEGEEACKK | 35 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVRPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 T114R | MIKPTVSQFLTPGFKLIRNHSRTAGLKLKNEGEEACKK | 36 |
| | FVRENEIPKDECPNFQGGPAIANIIAKSREFTEWEIYQSS | |
| | LAIQEVIFTLPKDKLPEPILKEEWRAQWLSEHGLDRVP | |
| | YKEAAGLNLIIKNAVNTYKGVQVKVDNKNKNNLAKIN | |
| | RKNEIAKLNGEQEISFEEIKAFDDKGYLLQKPSPNKSIY | |
| | CYQSVSPKPFITSKYHNVNLPEEYIGYYRKSNEPIVSPY | |
| | QFDRLRIPIGEPGYVPKWQYTFLSKKENKRRKLSKRIK | |
| | NVSPILGIICIKKDWCVFDMRGLLRTNHWKKYHKPTDS | |
| | INDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 D369A | MIKPTVSQFLTPGFKLIRNHSRTAGLKLKNEGEEACKK | 37 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVAVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 D369N | MIKPTVSQFLTPGFKLIRNHSRTAGLKLKNEGEEACKK | 38 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVNVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |
| CasPhi.12 D658A | MIKPTVSQFLTPGFKLIRNHSRTAGLKLKNEGEEACKK | 39 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNAAIDVATENLATVAITAQSMPKPTCERSGDAK | |
| | KPVRARKAKAPEFHDKLAPSYTVVLREAV | |
| CasPhi.12 D658N | MIKPTVSQFLTPGFKLIRNHSRTAGLKLKNEGEEACKK | 40 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIENLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNANIDVATENLATVAITAQSMPKPTCERSGDAK | |
| | KPVRARKAKAPEFHDKLAPSYTVVLREAV | |
| j12_L17_18_del1 | MIKPTVSQFLTPGFKLIRNHSRTAGLKLKNEGEEACKK | 41 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQGSSGDYKWFQDYKPKLS | |
| | KEVRDALSDIEWRLRRESLEFNKLSKSREQDARQLAN | |
| | WISSMCDVIGIENLVKKNNFFGGSGKREPGWDNFYKP | |
| | KKENRWWINAIHKALTELSQNKGKRVILLPAMRTSITC | |
| | PKCKYCDSKNRNGEKFNCLKCGIELNADIDVATENLAT | |
| | VAITAQSMPKPTCERSGDAKKPVRARKAKAPEFHDKL | |
| | APSYTVVLREAV | |
| j12_L17_18_del2 | MIKPTVSQFLTPGFKLIRNHSRTAGLKLKNEGEEACKK | 42 |
| | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEGSSGDYKWFQD | |
| | YKPKLSKEVRDALSDIEWRLRRESLEFNKLSKSREQDA | |
| | RQLANWISSMCDVIGIENLVKKNNFFGGSGKREPGWD | |
| | NFYKPKKENRWWINAIHKALTELSQNKGKRVILLPAM | |
| | RTSITCPKCKYCDSKNRNGEKFNCLKCGIELNADIDVA | |
| | TENLATVAITAQSMPKPTCERSGDAKKPVRARKAKAP | |
| | EFHDKLAPSYTVVLREAV | |
| CasPhi.12-L26K- | MIKPTVSQFLTPGFKLIRNHSRTAGKKLKNEGEEACKK | 43 |
| E567Q | FVRENEIPKDECPNFQGGPAIANIIAKSREFTEWEIYQSS | |
| | LAIQEVIFTLPKDKLPEPILKEEWRAQWLSEHGLDTVPY | |
| | KEAAGLNLIIKNAVNTYKGVQVKVDNKNKNNLAKINR | |
| | KNEIAKLNGEQEISFEEIKAFDDKGYLLQKPSPNKSIYC | |
| | YQSVSPKPFITSKYHNVNLPEEYIGYYRKSNEPIVSPYQ | |
| | FDRLRIPIGEPGYVPKWQYTFLSKKENKRRKLSKRIKN | |
| | VSPILGIICIKKDWCVFDMRGLLRTNHWKKYHKPTDSI | |
| | NDLFDYFTGDPVIDTKANVVRFRYKMENGIVNYKPVR | |
| | EKKGKELLENICDQNGSCKLATVDVGQNNPVAIGLFEL | |
| | KKVNGELTKTLISRHPTPIDFCNKITAYRERYDKLESSIK | |
| | LDAIKQLTSEQKIEVDNYNNNFTPQNTKQIVCSKLNINP | |
| | NDLPWDKMISGTHFISEKAQVSNKSEIYFTSTDKGKTK | |
| | DVMKSDYKWFQDYKPKLSKEVRDALSDIEWRLRRESL | |
| | EFNKLSKSREQDARQLANWISSMCDVIGIQNLVKKNNF | |
| | FGGSGKREPGWDNFYKPKKENRWWINAIHKALTELSQ | |
| | NKGKRVILLPAMRTSITCPKCKYCDSKNRNGEKFNCLK | |
| | CGIELNADIDVATENLATVAITAQSMPKPTCERSGDAK | |
| | KPVRARKAKAPEFHDKLAPSYTVVLREAV | |

TABLE 5
Exemplary Amino Acid Alterations Relative to CasM.265466 of SEQ ID NO: 3

| Effects | Amino Acid Alterations |
|---|---|
| | At least one substitution (i.e., with R, K or H) selected from K58, |
| | I80, T84, K105, N193, C202, S209, G210, A218, D220, E225, |
| | C246, N286, M295, M298, A306, Y315, and Q360 |
| Enhanced nuclease | I80R, T84R, K105R, C202R, G210R, A218R, D220R, E225R, |
| activity relative to the | C246R, Q360R, I80K, T84K, G210K, N193K, C202K, A218K, |
| wild-type effector protein | D220K, E225K, C246K, N286K, A306K, Q360K, I80H, T84H, |
| | K105H, G210H, C202H, A218H, D220H, E225H, C246H, |
| | Q360H, K58W, S209F, M295W, M298L, Y315M |
| | Double mutations: D220R/A306K, D220R/K250N |
| Reduced or abolished | D237A, D418A, D418N, E335A, E335Q |
| nuclease activity relative | |
| to the wild-type effector | |
| protein | |

TABLE 6
Exemplary Engineered CasM.265466 Effector Protein Variants

| Effector | | |
|---|---|---|
| Protein | Amino Acid Sequence | SEQ ID NO: |
| CasM.2654 | MSVLTRKVQLIPVGDKEERDRVYKYLRDGIEAQNRAMNLYM | 44 |
| 66 D220R | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMRIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 45 |
| 66 K58W | SGLYFAAINEASKEDRWELNQLYSRIATSSKGSAYTTDIEFPT | |
| | GLASTSTLSMAVRQDFTKSLKDGLMYGRVSLPTYRKDNPLFV | |
| | DVRFVALRGTKQKYNGLYHEYKSHTEFLDNLYSSDLKVYIKF | |
| | ANDITFQVIFGNPRKSSALRSEFQNIFEEYYKVCQSSIQFSGTKI | |
| | ILNMAMDIPDKEIELDEDVCVGVDLGIAIPAVCALNKNRYSR | |
| | VSIGSKEDFLRVRTKIRNQRKRLQTNLKSSNGGHGRKKKMKP | |
| | MDRFRDYEANWVQNYNHYVSRQVVDFAVKNKAKYINLENL | |
| | EGIRDDVKNEWLLSNWSYYQLQQYITYKAKTYGIEVRKINPY | |
| | HTSQRCSCCGYEDAGNRPKKEKGQAYFKCLKCGEEMNADFN | |
| | AARNIAMSTEFQSGKKTKKQKKEQHENK | |
| CasM.2654 | MSVLTRKVQLIPVGDKEERDRVYKYLRDGIEAQNRAMNLYM | 46 |
| 66 A218K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMKMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 47 |
| 66 M295W | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKWKP | |
| | MDRFRDYEANWVQNYNHYVSRQVVDFAVKNKAKYINLENL | |
| | EGIRDDVKNEWLLSNWSYYQLQQYITYKAKTYGIEVRKINPY | |
| | HTSQRCSCCGYEDAGNRPKKEKGQAYFKCLKCGEEMNADFN | |
| | AARNIAMSTEFQSGKKTKKQKKEQHENK | |
| CasM.2654 | MSVLTRKVQLIPVGDKEERDRVYKYLRDGIEAQNRAMNLYM | 48 |
| 66 M298L | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPL | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 49 |
| 66 N193K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQKIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 50 |
| 66 Y315M | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHMVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 51 |
| 66 S209F | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFFGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 52 |
| 66 I80K | SGLYFAAINEASKEDRKELNQLYSRIATSSKGSAYTTDKEFPT | |
| | GLASTSTLSMAVRQDFTKSLKDGLMYGRVSLPTYRKDNPLFV | |
| | DVRFVALRGTKQKYNGLYHEYKSHTEFLDNLYSSDLKVYIKF | |
| | ANDITFQVIFGNPRKSSALRSEFQNIFEEYYKVCQSSIQFSGTKI | |
| | ILNMAMDIPDKEIELDEDVCVGVDLGIAIPAVCALNKNRYSR | |
| | VSIGSKEDFLRVRTKIRNQRKRLQTNLKSSNGGHGRKKKMKP | |
| | MDRFRDYEANWVQNYNHYVSRQVVDFAVKNKAKYINLENL | |
| | EGIRDDVKNEWLLSNWSYYQLQQYITYKAKTYGIEVRKINPY | |
| | HTSQRCSCCGYEDAGNRPKKEKGQAYFKCLKCGEEMNADFN | |
| | AARNIAMSTEFQSGKKTKKQKKEQHENK | |
| CasM.2654 | MSVLTRKVQLIPVGDKEERDRVYKYLRDGIEAQNRAMNLYM | 53 |
| 66 E225K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKKIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 54 |
| 66 N286K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSKGGHGRKKKMKP | |
| | MDRFRDYEANWVQNYNHYVSRQVVDFAVKNKAKYINLENL | |
| | EGIRDDVKNEWLLSNWSYYQLQQYITYKAKTYGIEVRKINPY | |
| | HTSQRCSCCGYEDAGNRPKKEKGQAYFKCLKCGEEMNADEN | |
| | AARNIAMSTEFQSGKKTKKQKKEQHENK | |
| CasM.2654 | MSVLTRKVQLIPVGDKEERDRVYKYLRDGIEAQNRAMNLYM | 55 |
| 66 A306K | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEKNWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 56 |
| 66 E335Q | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLQNLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 57 |
| 66 D237A | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVALGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 58 |
| 66 D418A | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNAAFNA | |
| | ARNIAMSTEFQSGKKTKKQKKEQHENK | |
| CasM.2654 | MSVLTRKVQLIPVGDKEERDRVYKYLRDGIEAQNRAMNLYM | 59 |
| 66 D418N | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLENLE | |
| | GIRDDVKNEWLLSNWSYYQLQQYITYKAKTYGIEVRKINPYH | |
| | TSQRCSCCGYEDAGNRPKKEKGQAYFKCLKCGEEMNANFNA | |
| | ARNIAMSTEFQSGKKTKKQKKEQHENK | |
| CasM.2654 | MSVLTRKVQLIPVGDKEERDRVYKYLRDGIEAQNRAMNLYM | 60 |
| 66 E335A | SGLYFAAINEASKEDRKELNQLYSRIATSSKGSAYTTDIEFPTG | |
| | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMDIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLANLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |
| CasM.2654 | MSVLTRKVQLIPVGDKEERDRVYKYLRDGIEAQNRAMNLYM | 61 |
| 66 D220R- | SGLYFAAINEASKEDRKELNQLYSRIATSSKGSAYTTDIEFPTG | |
| E335Q | LASTSTLSMAVRQDFTKSLKDGLMYGRVSLPTYRKDNPLFVD | |
| | VRFVALRGTKQKYNGLYHEYKSHTEFLDNLYSSDLKVYIKFA | |
| | NDITFQVIFGNPRKSSALRSEFQNIFEEYYKVCQSSIQFSGTKII | |
| | LNMAMRIPDKEIELDEDVCVGVDLGIAIPAVCALNKNRYSRV | |
| | SIGSKEDFLRVRTKIRNQRKRLQTNLKSSNGGHGRKKKMKPM | |
| | DRFRDYEANWVQNYNHYVSRQVVDFAVKNKAKYINLQNLE | |
| | GIRDDVKNEWLLSNWSYYQLQQYITYKAKTYGIEVRKINPYH | |
| | TSQRCSCCGYEDAGNRPKKEKGQAYFKCLKCGEEMNADFNA | |
| | ARNIAMSTEFQSGKKTKKQKKEQHENK | |

TABLE 7
Exemplary base editing enzymes,
base editor fusion proteins, and linkers

| Protein | Amino Acid Sequence | SEQ ID NO: |
|---|---|---|
| ABE8e | SEVEFSHEYWMRHALTLAKRARDEREVPVGAVLVLNNR | 64 |
| | VIGEGWNRAIGLHDPTAHAEIMALRQGGLVMQNYRLIDA | |
| | TLYVTFEPCVMCAGAMIHSRIGRVVFGVRNSKRGAAGSL | |
| | MNVLNYPGMNHRVEITEGILADECAALLCDFYRMPRQVF | |
| | NAQKKAQSSIN | |
| CasM.265466- | MSVLTRKVQLIPVGDKEERDRVYKYLRDGIEAQNRAMNL | 65 |
| D220R- | YMSGLYFAAINEASKEDRKELNQLYSRIATSSKGSAYTTDI | |
| E335Q_ABE8e | EFPTGLASTSTLSMAVRQDFTKSLKDGLMYGRVSLPTYRK | |
| fusion | DNPLFVDVRFVALRGTKQKYNGLYHEYKSHTEFLDNLYS | |
| | SDLKVYIKFANDITFQVIFGNPRKSSALRSEFQNIFEEYYKV | |
| | CQSSIQFSGTKIILNMAMRIPDKEIELDEDVCVGVDLGIAIP | |
| | AVCALNKNRYSRVSIGSKEDFLRVRTKIRNQRKRLQTNLK | |
| | SSNGGHGRKKKMKPMDRFRDYEANWVQNYNHYVSRQV | |
| | VDFAVKNKAKYINLQNLEGIRDDVKNEWLLSNWSYYQL | |
| | QQYITYKAKTYGIEVRKINPYHTSQRCSCCGYEDAGNRPK | |
| | KEKGQAYFKCLKCGEEMNADFNAARNIAMSTEFQSGKKT | |
| | KKQKKEQHENKGSSGGSPAGSPTSTEEGTSESATPESGPGT | |
| | STEPSEGSAPGSPAGSGGGSSEVEFSHEYWMRHALTLAKR | |
| | ARDEREVPVGAVLVLNNRVIGEGWNRAIGLHDPTAHAEI | |
| | MALRQGGLVMQNYRLIDATLYVTFEPCVMCAGAMIHSRI | |
| | GRVVFGVRNSKRGAAGSLMNVLNYPGMNHRVEITEGILA | |
| | DECAALLCDFYRMPRQVFNAQKKAQSSIN | |
| CasPhi.12- | MIKPTVSQFLTPGFKLIRNHSRTAGKKLKNEGEEACKKFV | 66 |
| L26K- | RENEIPKDECPNFQGGPAIANIIAKSREFTEWEIYQSSLAIQ | |
| E567Q_ABE8e | EVIFTLPKDKLPEPILKEEWRAQWLSEHGLDTVPYKEAAG | |
| fusion | LNLIIKNAVNTYKGVQVKVDNKNKNNLAKINRKNEIAKL | |
| | NGEQEISFEEIKAFDDKGYLLQKPSPNKSIYCYQSVSPKPFI | |
| | TSKYHNVNLPEEYIGYYRKSNEPIVSPYQFDRLRIPIGEPGY | |
| | VPKWQYTFLSKKENKRRKLSKRIKNVSPILGIICIKKDWCV | |
| | FDMRGLLRTNHWKKYHKPTDSINDLFDYFTGDPVIDTKA | |
| | NVVRFRYKMENGIVNYKPVREKKGKELLENICDQNGSCK | |
| | LATVDVGQNNPVAIGLFELKKVNGELTKTLISRHPTPIDFC | |
| | NKITAYRERYDKLESSIKLDAIKQLTSEQKIEVDNYNNNFT | |
| | PQNTKQIVCSKLNINPNDLPWDKMISGTHFISEKAQVSNKS | |
| | EIYFTSTDKGKTKDVMKSDYKWFQDYKPKLSKEVRDALS | |
| | DIEWRLRRESLEFNKLSKSREQDARQLANWISSMCDVIGI | |
| | QNLVKKNNFFGGSGKREPGWDNFYKPKKENRWWINAIH | |
| | KALTELSQNKGKRVILLPAMRTSITCPKCKYCDSKNRNGE | |
| | KFNCLKCGIELNADIDVATENLATVAITAQSMPKPTCERS | |
| | GDAKKPVRARKAKAPEFHDKLAPSYTVVLREAVGSSGGS | |
| | PAGSPTSTEEGTSESATPESGPGTSTEPSEGSAPGSPAGSGG | |
| | GSSEVEFSHEYWMRHALTLAKRARDEREVPVGAVLVLNN | |
| | RVIGEGWNRAIGLHDPTAHAEIMALRQGGLVMQNYRLID | |
| | ATLYVTFEPCVMCAGAMIHSRIGRVVFGVRNSKRGAAGS | |
| | LMNVLNYPGMNHRVEITEGILADECAALLCDFYRMPRQV | |
| | FNAQKKAQSSIN | |
| Linker | GSSGGSPAGSPTSTEEGTSESATPESGPGTSTEPSEGSAPGS | 67 |
| | PAGSGGGS | |

TABLE 8
Exemplary PAM TTN Sequences

| | PAM Sequence |
|---|---|
| PAM # | (5′-3′) |
| 1 | TTG |
| 2 | TTC |
| 3 | TTT |
| 4 | TTA |

TABLE 9
Exemplary PAM NNTN Sequences

| PAM # | PAM Sequence (5′-3′) |
|---|---|
| 1 | TTTG |
| 2 | TCTG |
| 3 | TGTG |
| 4 | TCTA |
| 5 | TATA |
| 6 | TTTA |
| 7 | TGTA |
| 8 | TATG |

TABLE 10
Exemplary Spacer Sequences

| ID# | Spacer sequence | Cas protein | Target allele | SEQ ID NO: |
|---|---|---|---|---|
| R13988 | UUGUCCAGCU | CasPhi/CasPhi L26R | NRAS WT | 68 |
| | GUAUCCAGUA | | | |
| R13989 | UUGUCCAGCU | CasPhi/CasPhi L26R | NRAS WT | 69 |
| | GUAUCCAGU | | | |
| R13990 | UUGUCCAGCU | CasPhi/CasPhi L26R | NRAS WT | 70 |
| | GUAUCCAG | | | |
| | UUGUCCAGCU | CasPhi/CasPhi L26R | NRAS WT | 71 |
| | GUAUCCA | | | |
| R14000 | UAGUCCAGCU | CasPhi/CasPhi L26R | NRAS Q61L | 72 |
| | GUAUCCAGUA | | | |
| R14001 | UAGUCCAGCU | CasPhi/CasPhi L26R | NRAS Q61L | 73 |
| | GUAUCCAGU | | | |
| R14002 | UAGUCCAGCU | CasPhi/CasPhi L26R | NRAS Q61L | 74 |
| | GUAUCCAG | | | |
| | UAGUCCAGCU | CasPhi/CasPhi L26R | NRAS Q61L | 75 |
| | GUAUCCA | | | |
| R13833 | CUCUUCUUGU | CasM.265466 | NRAS WT | 76 |
| | CCAGCUGUAU | | | |
| R13834 | CUCUUCUAGU | CasM.265466 | NRAS Q61L | 77 |
| | CCAGCUGUAU | | (mutation in PAM + 8) | |
| R13835 | GUCCAGCUGU | CasM.265466 | NRAS Q61L | 78 |
| | AUCCAGUAUG | | (mutation in PAM) | |
| R5681 | GUAGUUGGAG | CasM.265466 | KRAS WT | 79 |
| | CUGGUGGCGU | | | |
| R5682 | GUAGUUGGAG | CasM.265466 | KRAS G12D | 80 |
| | CUGAUGGCGU | | (mutation in PAM + 14) | |

TABLE 11
Exemplary Repeat Sequences

| Repeat sequence | | SEQ |
|---|---|---|
| (shown as RNA), 5′-3′ | CAS protein | ID NO: |
| CUUUCAAGACUAAUAGAUUGCUCCUUACGA | CasPhi.12 | 81 |
| GGAGAC | | |
| AUAGAUUGCUCCUUACGAGGAGAC | CasPhi.12 | 82 |
| UAGAUUGCUCCUUACGAGGAGAC | CasPhi.12 | 83 |
| AGAUUGCUCCUUACGAGGAGAC | CasPhi.12 | 84 |
| GAUUGCUCCUUACGAGGAGAC | CasPhi.12 | 85 |
| AUUGCUCCUUACGAGGAGAC | CasPhi.12 | 86 |
| GUAGAUUGCUCCUUACGAGGAGAC | CasPhi.12 | 87 |
| AGACUAAUAGAUUGCUCCUUACGAGGAGAC | CasPhi.12 | 88 |
| AAGGAUGCCAAAC | CasM.265466 | 89 |

TABLE 12
Exemplary Full Guide Sequences for
CasPhi.12 Effector Protein and
Engineered CasPhi.12 Effector Protein Variants

| Target | Guide sequence | SEQ |
|---|---|---|
| Allele | (shown as RNA), (5′ to 3′) | ID NO: |
| NRAS WT | CUUUCAAGACUAAUAGAUUGCUCCUUACGA | 95 |
| | GGAGACUUGUCCAGCUGUAUCCAGUA | |
| NRAS WT | CUUUCAAGACUAAUAGAUUGCUCCUUACGA | 96 |
| | GGAGACUUGUCCAGCUGUAUCCAGU | |
| NRAS WT | AUUGCUCCUUACGAGGAGACUUGUCCAGCU | 97 |
| CCAG | GUAUCCAG | |
| NRAS WT | AUUGCUCCUUACGAGGAGACUUGUCCAGCU | 98 |
| | GUAUCCA | |
| NRAS Q61L | CUUUCAAGACUAAUAGAUUGCUCCUUACGA | 99 |
| | GGAGACUAGUCCAGCUGUAUCCAGUA | |
| NRAS Q61L | CUUUCAAGACUAAUAGAUUGCUCCUUACGA | 100 |
| | GGAGACUAGUCCAGCUGUAUCCAGU | |
| NRAS Q61L | AUUGCUCCUUACGAGGAGACUAGUCCAGCU | 101 |
| | GUAUCCAG | |
| NRAS Q61L | AUUGCUCCUUACGAGGAGACUAGUCCAGCU | 102 |
| | GUAUCCA | |

TABLE 13
Exemplary Full Guide Sequences for CasM.265466
Effector Protein and
Engineered CasM.265466 Effector Protein Variants

| Target | Guide sequence | SEQ |
|---|---|---|
| Allele | (shown as RNA), (5′ to 3′) | ID NO: |
| NRAS WT | ACAGCUUAUUUGGAAGCUGAAAUGUGAGGUUUA | 103 |
| | UAACACUCACAAGAAUCCUGAAAAAGGAUGCCA | |
| | AACCUCUUCUUGUCCAGCUGUAU | |
| NRAS Q61L | ACAGCUUAUUUGGAAGCUGAAAUGUGAGGUUUA | 104 |
| | UAACACUCACAAGAAUCCUGAAAAAGGAUGCCA | |
| | AACCUCUUCUAGUCCAGCUGUAU | |
| NRAS Q61L | ACAGCUUAUUUGGAAGCUGAAAUGUGAGGUUUA | 105 |
| | UAACACUCACAAGAAUCCUGAAAAAGGAUGCCA | |
| | AAGUCCAGCUGUAUCCAGUAUG | |
| KRAS WT | ACAGCUUAUUUGGAAGCUGAAAUGUGAGGUUUA | 106 |
| | UAACACUCACAAGAAUCCUGAAAAAGGAUGCCA | |
| | AACGUAGUUGGAGCUGGUGGCGU | |
| KRAS G12D | ACAGCUUAUUUGGAAGCUGAAAUGUGAGGUUUA | 107 |
| | UAACACUCACAAGAAUCCUGAAAAAGGAUGCCA | |
| | AACGUAGUUGGAGCUGAUGGCGU | |

TABLE 14
Exemplary Modified Full Guide Sequences for
CasPhi.12 Effector Protein and
Engineered CasPhi.12 Effector Protein Variants

| Target | Guide sequence | SEQ |
|---|---|---|
| Allele | (shown as RNA), (5′ to 3′) | ID NO: |
| NRAS WT | mC*mU*mU*UCAAGACUAAUAGAUUGCUCCUUAC | 108 |
| | GAGGAGACUUGUCCAGCUGUAUCCAmG*mU*mA | |
| NRAS WT | mC*mU*mU*UCAAGACUAAUAGAUUGCUCCUUAC | 109 |
| | GAGGAGACUUGUCCAGCUGUAUCCmA*mG*mU | |
| NRAS WT | mC*mU*mU*UCAAGACUAAUAGAUUGCUCCUUAC | 110 |
| | GAGGAGACUUGUCCAGCUGUAUCmC*mA*mG | |
| NRAS Q61L | mC*mU*mU*UCAAGACUAAUAGAUUGCUCCUUAC | 111 |
| | GAGGAGACUAGUCCAGCUGUAUCCAmG*mU*mA | |
| NRAS Q61L | mC*mU*mU*UCAAGACUAAUAGAUUGCUCCUUAC | 112 |
| | GAGGAGACUAGUCCAGCUGUAUCCmA*mG*mU | |
| NRAS Q61L | mC*mU*mU*UCAAGACUAAUAGAUUGCUCCUUAC | 113 |
| | GAGGAGACUAGUCCAGCUGUAUCmC*mA*mG | |

Notes:
m is 2′ O-Me modified sugar moiety; and the * denotes a
PS linkage.

TABLE 15
Exemplary Modified Full Guide Sequences
for CasM.265466 Effector Protein and Engineered
CasM.265466 Effector Protein Variants

| | | SEQ |
|---|---|---|
| Target | Guide sequence | ID |
| Allele | (shown as RNA), (5′ to 3′) | NO: |
| NRAS WT | mA*mC*mA*GCUUAUUUGGAAGCUGAAAUGUGAG | 114 |
| | GUUUAUAACACUCACAAGAAUCCUGAAAAAGGAU | |
| | GCCAAACCUCUUCUUGUCCAGCUGmU*mA*mU | |
| NRAS Q61L | mA*mC*mA*GCUUAUUUGGAAGCUGAAAUGUGAG | 115 |
| | GUUUAUAACACUCACAAGAAUCCUGAAAAAGGAU | |
| | GCCAAACCUCUUCUAGUCCAGCUGmU*mA*mU | |
| NRAS Q61L | mA*mC*mA*GCUUAUUUGGAAGCUGAAAUGUGAG | 116 |
| | GUUUAUAACACUCACAAGAAUCCUGAAAAAGGAU | |
| | GCCAAAGUCCAGCUGUAUCCAGUmA*mU*mG | |
| KRAS WT | mA*mC*mA*GCUUAUUUGGAAGCUGAAAUGUGAG | 117 |
| | GUUUAUAACACUCACAAGAAUCCUGAAAAAGGAU | |
| | GCCAAACGUAGUUGGAGCUGGUGGmC*mG*mU | |
| KRAS | mA*mC*mA*GCUUAUUUGGAAGCUGAAAUGUGAG | 118 |
| G12D | GUUUAUAACACUCACAAGAAUCCUGAAAAAGGAU | |
| | GCCAAACGUAGUUGGAGCUGAUGGmC*mG*mU | |

Notes:
m is 2′ O-Me modified sugar moiety; and the * denotes a
PS linkage.

### EXAMPLES

### Example 1: Allele-specific NRAS Knockdown Using CasPhi.12, CasPhi.12 L26R and CasM.265466

Experiments were carried out to assess the specificity of CasPhi.12 (SEQ ID NO: 1), CasPhi.12 L26R (SEQ ID NO: 24), and CasM.265466 (SEQ ID NO: 3) in knockdown of the NRAS mutant allele in a human melanoma cell line HepG2 by measuring indel activity and colony formation.

The HepG2 human melanoma cell line is a heterozygous cell line that harbors one copy of NRAS mutant allele encoding a NRAS protein with the Q61L mutation and one copy of the NRAS wildtype (WT) allele. Hep3B is a control cell line with homozygous WT NRAS.

HepG2 or control Hep3B cells were transfected with mRNA encoding the CasPhi12 nuclease, the CasPhi.12 L26R nuclease, or the CasM.265466 and guide nucleic acid at a 5 μg:500 pmol ratio. One set of transfected cells were then grown for 48 hours and the indel activity of the effector protein/guide nucleic acid was analyzed by next-generation sequencing (NGS). The other set of transfected cells were incubated for 15 days for colony formation assessment.

The nuclease mRNA encodes CasPhi.12 (SEQ ID NO: 1), CasPhi.12 L26R (SEQ ID NO: 24), or CasM.265466 (SEQ ID NO: 3). The guide nucleic acids were designed to target either the WT allele or mutant allele containing the c.182 A>T mutation. The sequence of NRAS WT target locus is GGACATACTGGATACAGCTGGACAAGAA (SEQ ID NO: 93). The sequence of NRAS Q61L target locus (mutation position c.182 A>T) is GGACATACTGGATACAGCTGGACTAGAA (SEQ ID NO: 94).

Three types of guide nucleic acids were tested with CasPhi.12 or CasPhi.12 L26R as follows:
(1) a guide nucleic acid targeting the WT allele (CasPhi.12 NRAS WT guide);
(2) a guide nucleic acid containing a mutation in the 2^{nd} nucleotide in the spacer sequence and targeting the Q61L mutant allele (CasPhi.12 NRAS Q61L guide); and
(3) a guide nucleic acid containing no mutation in the spacer sequence and targeting a target sequence adjacent to the WT PAM sequence (TTG) that is only in the WT allele (CasPhi.12 WT PAM guide).

TABLE 16 provides exemplary guide nucleic acids tested with the CasPhi.12 and CasPhi.12 L26R nucleases. These guide nucleic acids demonstrated high allele-specificity and efficiency in suppressing proliferation, i.e., knockout of the NRAS Q61L allele in HepG2 cells reduced proliferation.

TABLE 16
Exemplary guide nucleic acids for
CasPhi.12 and CasPhi.12 L26R

| | Target | | Repeat-Spacer |
|---|---|---|---|
| Name | allele | Spacer Sequence | length |
| R13990 | NRAS WT | UUGUCCAGCUGUAUCCAG | 36-18 |
| | | (SEQ ID NO: 70) | |
| R14002 | NRAS Q61L | UAGUCCAGCUGUAUCCAG | 36-18 |
| | | (SEQ ID NO: 74) | |

Three types of guide nucleic acids were tested with CasM.265466 as follows:
(1) a guide nucleic acid targeting the NRAS WT allele (CasM.265466 NRAS WT guide);
(2) a guide nucleic acid containing a mutation in the 8^{th} nucleotide in the spacer sequence and targeting the Q61L mutant allele (CasM.265466 NRAS Q61L guide); and
(3) a guide nucleic acid containing no mutation in the spacer sequence and targeting a target sequence adjacent to the mutated PAM sequence (TCTA) that is only in the Q61L allele (CasM.265466 Q61L PAM guide).

TABLE 17 provides exemplary guide nucleic acids tested with CasM265466 nuclease. These guide nucleic acids demonstrated high allele-specificity and efficiency in suppressing proliferation.

TABLE 17
Exemplary guide nucleic acids for CasM265466

| Name | Targeting allele | Spacer Sequence | PAM |
|---|---|---|---|
| R13833 | NRAS WT | CUCUUCUUGUCCAGC | TGTA |
| | | UGUAU | |
| | | (SEQ ID NO: 76) | |
| R13834 | NRAS Q61L | CUCUUCUAGUCCAGC | TGTA |
| | | UGUAU | |
| | (mutation in PAM + 8) | (SEQ ID NO: 77) | |
| R13835 | NRAS Q61L | GUCCAGCUGUAUCCA | TCTA |
| | (mutation in PAM) | GUAUG | |
| | | (SEQ ID NO: 78) | |

Handle sequence for CasM265466 (69 nt):
ACAGCUUAUUUGGAAGCUGAAAUGUGAGGUUUAUAACACUC
ACAAGAAUCCUGAAAAAGGAUGCCAAAC

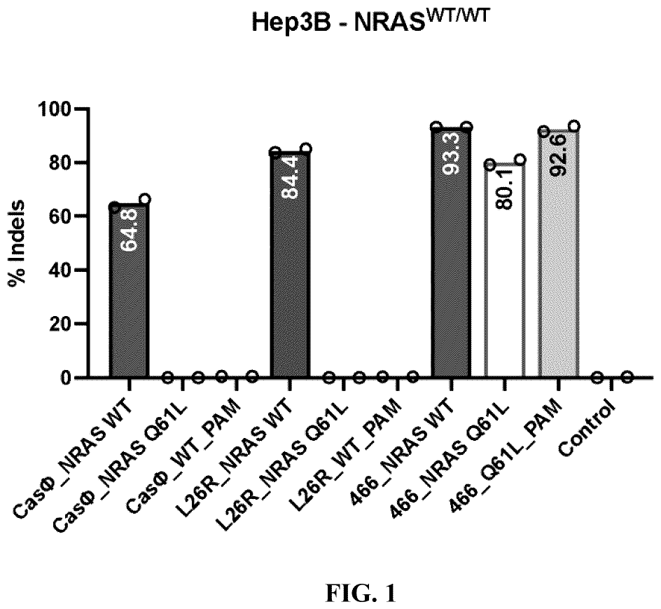

FIG. 1 shows NRAS knockdown in the Hep3B control cells. CasPhi.12 and CasPhi.12 L26R, when paired with the WT guide, generated more than 60% Indel formation in the Hep3B cells. CasPhi.12 L26R performed better than CasPhi.12. The WT guide for CasPhi.12 and CasPhi.12 L26R was highly specific to the NRAS WT allele. The Q61L guide showed no observable activity in Hep3B cells. In contrast, CasM265466 showed high activity, yet it lacked specificity with the Q61L guide or Q61L PAM guide. When paired with the WT, Q61L, or Q61L PAM guide, CasM265466 all generated more than 80% Indel formation.

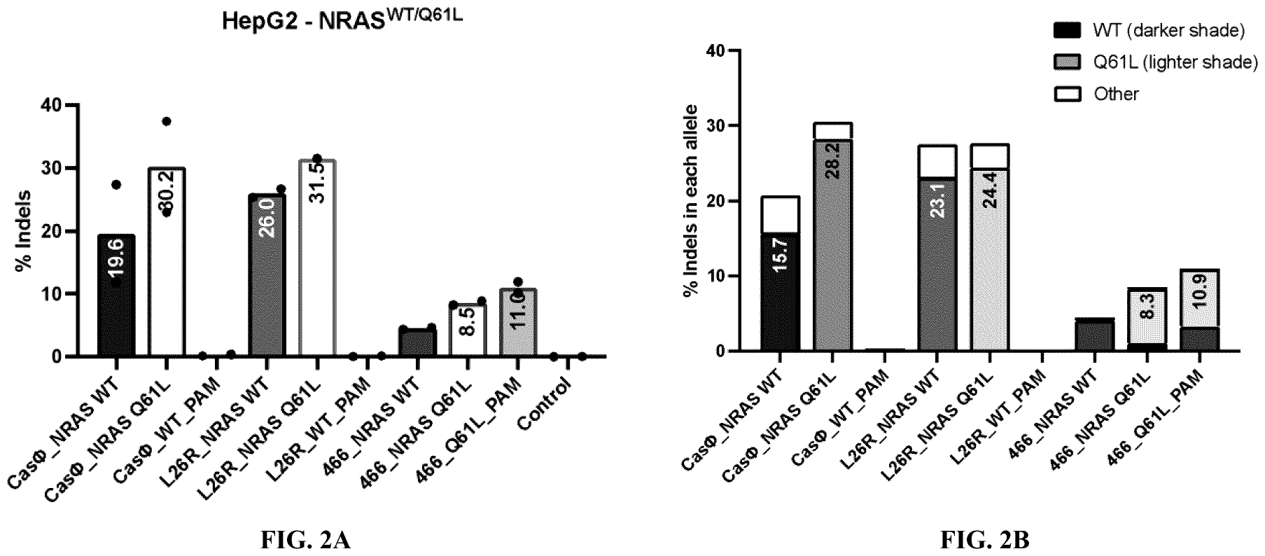

FIG. 2A and FIG. 2B show NRAS knockdown in the HepG2 heterozygous cells. As shown in FIG. 2A, CasPhi.12 and CasPhi.12 L26R, when paired with the Q61L guide, generated about 30% total Indel formation in the HepG2 cells. As shown in FIG. 2B, the Q61L guide for CasPhi.12 and CasPhi.12 L26R displayed high allele-specific activity. CasPhi.12 and CasPhi.12 L26R, when paired with the Q61L guide generated approximately 28.2% and 24.4% Indel formation in the Q61L allele, respectively. While CasM265466, when paired with the Q61L guide or Q61L PAM guide, showed moderate Indel formation activity in the HepG2 cells (FIG. 2A), it is important to note that both the Q61L guide and Q61L PAM guide were highly specific to the NRAS G12D allele (FIG. 2B).

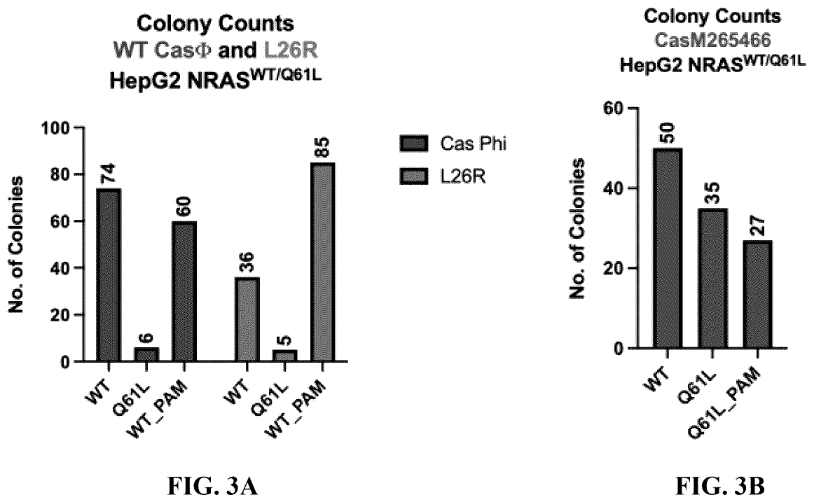

Colony formation assays were performed to assess proliferation of transfected HepG2 cells. The presence of colonies means that the proliferative capabilities of the cells have not been affected. 15 days after transfection, cells were washed and fixed. Fixed cells were then washed thoroughly with water. Images of plates were captured and stained cell colonies quantified. Results are shown in FIG. 3A and FIG. 3B. As shown in FIG. 3A, CasPhi.12 and CasPhi.12 L26R, when paired with the Q61L guide, resulted in the largest reduction in proliferation. However, cells continued to proliferate when CasPhi.12 and CasPhi.12 L26R paired with the WT or WT PAM guide. FIG. 3B shows that, CasM265466, when combined with the Q61L PAM guide, reduced proliferation the most, followed by the Q61L guide, with the WT guide having the smallest impact on proliferation.

The results indicate that CasPhi.12, CasPhi.12 L26R, and CasM.265466 are able to achieve allele-specific editing, enabling them to target and modify specific mutant alleles without affecting the wildtype alleles.

### Example 2: NRAS Guide Screen for CasPhi.12 L26R

This study aimed to evaluate how the lengths of repeat and spacer sequences affect the editing efficiency and specificity of guide RNAs when targeting the NRAS Q61L allele in homozygous and heterozygous conditions. The following repeat-spacer length combinations were tested in combination with the CasPhi.12 L26R nuclease to target the WT allele or Q61L mutant allele:
a. 36 nt repeat (SEQ ID NO: 81) with a 20nt, 19nt, 18nt, or 17nt spacer;
b. 30 nt repeat (SEQ ID NO: 88) with a 20nt, 19nt, 18nt, or 17nt spacer; or
c. 24 nt repeat (SEQ ID NO: 82) with a 20nt, 19nt, 18nt, or 17nt spacer.

The wildtype allele-specific spacer sequence of 18 nt has the sequence of SEQ ID NO: 70. Other wildtype allele-specific spacer sequences are based on the sequence of SEQ ID NO: 70 and have either one or two nucleotides added or subtracted. For example, the spacer sequences specific for the wildtype allele have the following sequences: SEQ ID NO: 68 for the 20-nt spacer, SEQ ID NO: 69 for the 19-nt spacer, and SEQ ID NO: 71 for the 17-nt spacer. The Q61L mutant allele-specific spacer sequence of 18 nt has the sequence of SEQ ID NO: 74. Other Q61L mutant allele-specific spacer sequences are based on the sequence of SEQ ID NO: 74 and have one or two nucleotides added or subtracted. For example, the spacer sequences specific for the Q61L mutant allele have the following sequences: SEQ ID NO: 72 for the 20-nt spacer, SEQ ID NO: 73 for the 19-nt spacer, and SEQ ID NO: 75 for the 17-nt spacer.

HepG2 cells or the control Hep3B cells were transfected with mRNA encoding nucleases and guide nucleic acid at a 5 μg:500 pmol ratio. The transfected cells were then grown for 48 hours and indel activity of the effector protein/guide nucleic acid was analyzed by NGS.

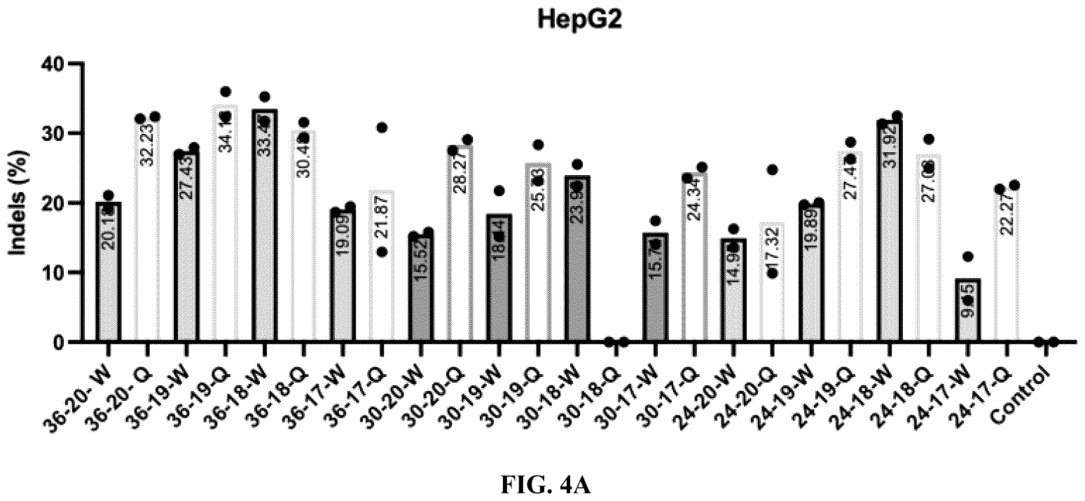

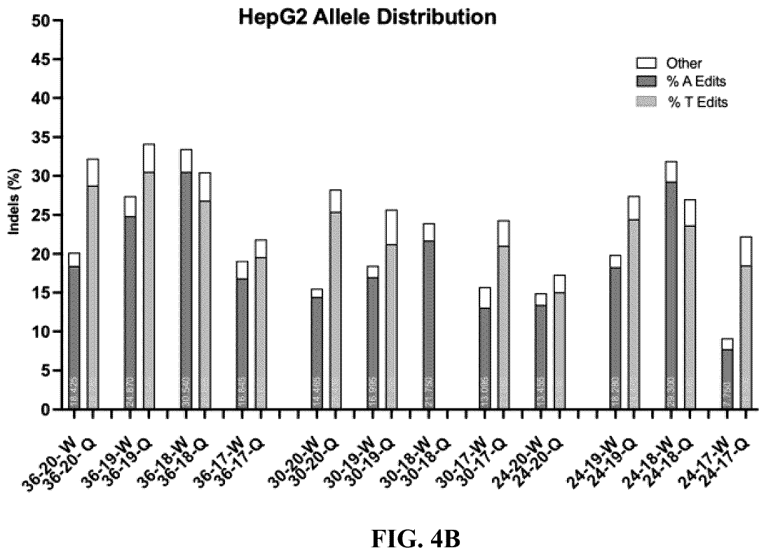

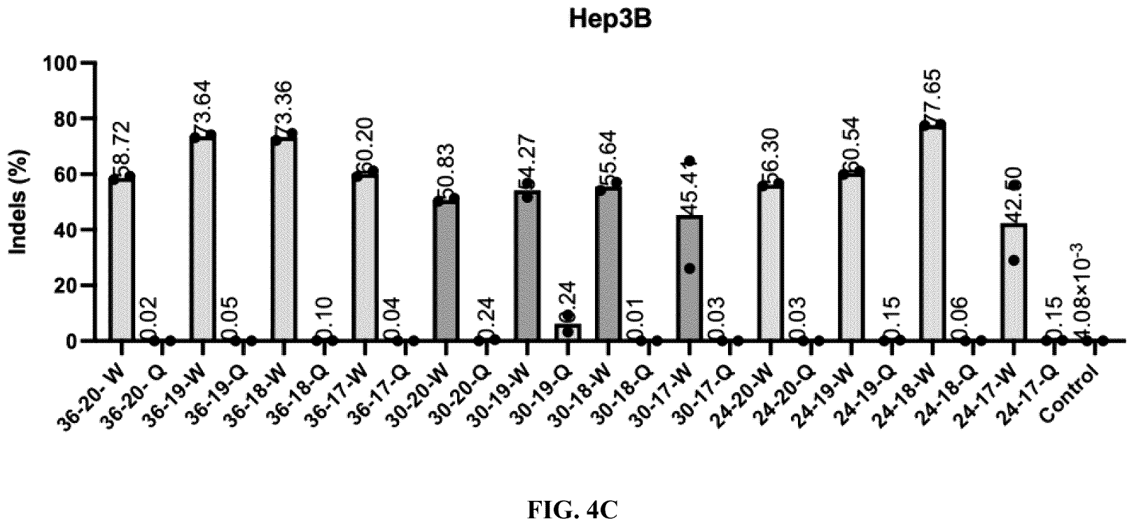

FIG. 4A shows that a repeat sequence length of 36 nucleotides, paired with spacer sequence lengths of 18, 19 or 20 nucleotides, exhibited both robust editing efficiency and high specificity in knocking out the NRAS Q61L allele in a heterozygous condition. FIG. 4B shows the WT and Q61L guides displayed high allele-specific activity in a heterozygous condition. FIG. 4C shows that the WT guides were highly specific to the NRAS WT locus in a homozygous WT condition. The Q61L guide showed no observable activity in a homozygous WT condition.

### Example 3: Allele-Specific Editing of the NRAS Gene Using Cas.Phi12 L26R and Modified Guide Nucleic Acids

This study aimed to evaluate the editing efficiency and specificity of Cas.Phi12 L26R when paired with modified guide nucleic acids.

The guide nucleic acids used in Example 2 for CasPhi.12 L26R were subject to either M1 or M6 modifications. The guide nucleic acids with the M1 modification contain 2′ O-Methyl modifications of the first 3 nucleotides at the 3′ end. The guide nucleic acids with the M6 modification contain 2′ O-Methyl modifications of the first 3 nucleotides and last 3 nucleotides, as well as phosphorothioate linkages between the first 4 nucleotides, and phosphorothioate linkages between the last 3 nucleotides. TABLE 18 provides the exemplary modified guide nucleic acids were tested with CasPhi.12 L26R.

TABLE 18
Exemplary modified guide nucleic
acids for CasPhi.12 L26R

| | Target | Guide sequence | SEQ |
|---|---|---|---|
| Name | allele | (shown as RNA), (5′ to 3′) | ID NO |
| R13988- | NRAS | mC*mU*mU*UCAAGACUAAUAGAUUGCUC | 108 |
| M6 | WT | CUUACGAGGAGACUUGUCCAGCUGUAUCC | |
| | | AmG*mU*mA | |
| R13989- | NRAS | mC*mU*mU*UCAAGACUAAUAGAUUGCUC | 109 |
| M6 | WT | CUUACGAGGAGACUUGUCCAGCUGUAUCC | |
| | | mA*mG*mU | |
| R13990- | NRAS | mC*mU*mU*UCAAGACUAAUAGAUUGCUC | 110 |
| M6 | WT | CUUACGAGGAGACUUGUCCAGCUGUAUCm | |
| | | C*mA*mG | |
| R14000- | NRAS | mC*mU*mU*UCAAGACUAAUAGAUUGCUC | 111 |
| M6 | Q61L | CUUACGAGGAGACUAGUCCAGCUGUAUCC | |
| | | AmG*mU*mA | |
| R14001- | NRAS | mC*mU*mU*UCAAGACUAAUAGAUUGCUC | 112 |
| M6 | Q61L | CUUACGAGGAGACUAGUCCAGCUGUAUCC | |
| | | mA*mG*mU | |
| R14002- | NRAS | mC*mU*mU*UCAAGACUAAUAGAUUGCUC | 113 |
| M6 | Q61L | CUUACGAGGAGACUAGUCCAGCUGUAUCm | |
| | | C*mA*mG | |

Notes:
M6 indicates that a guide contains M6 modifications; m is
2′ O-Me modified sugar moiety; and the * denotes a PS linkage

HepG2 or control Hep3B cells were transfected with nuclease mRNA and guide nucleic acid at a 5 μg:500 pmol ratio. The transfected cells were then grown for 48 hours and indel activity of the effector protein/guide nucleic acid was analyzed by NGS.

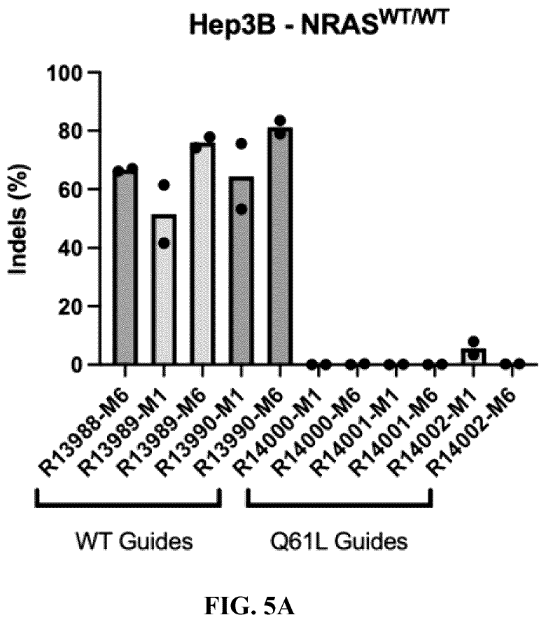

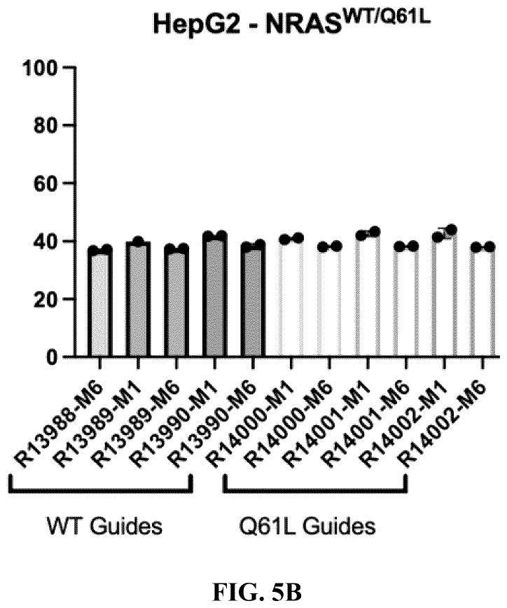

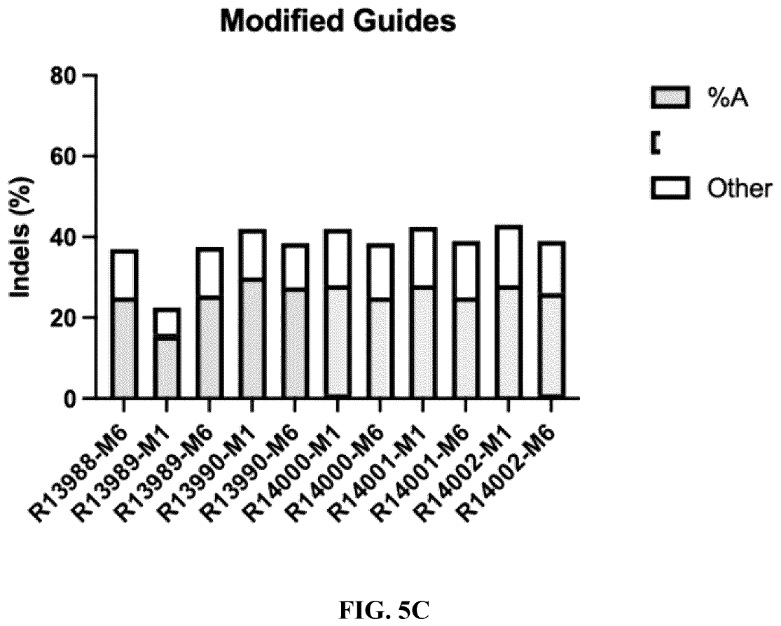

FIGS. 5A-5C show that the guides with modifications showed increased editing efficiency and remained allele-specific in HepG2 cells or Hep3B cells. There was negligible difference between the M1 and M6 modifications. The modified WT guide R13990-M6 and the modified Q61L guide R14002-M6 were selected for NRAS knockout.

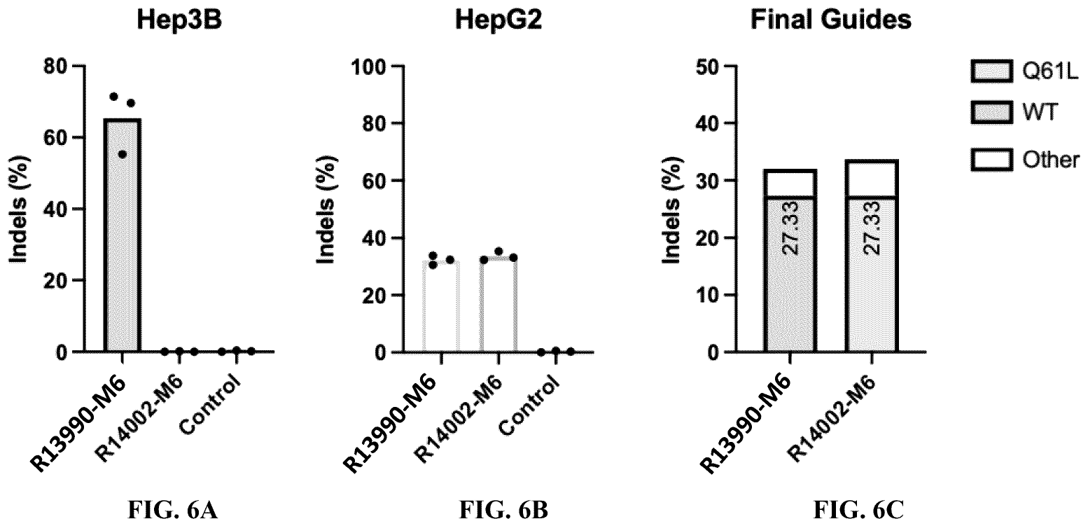

FIG. 6A shows that the modified WT guide R13990-M6 when combined with CasPhi.12 L26R generated more than 60% Indel formation in the Hep3B cells. The modified WT guide nucleic acid for CasPhi.12 L26R is highly specific to NRAS WT locus. The modified Q61L guide showed no observable activity. As shown in FIG. 6B and FIG. 6C, the modified WT and Q61L guides for CasPhi.12 L26R displayed high allele-specific activity in HepG2 cells.

### Example 4: Allele-Specific Editing of the NRAS Gene Using CasM265466 and Modified Guide Nucleic Acids

This study aimed to evaluate the editing efficiency and specificity of CasM265466 when paired with modified guide nucleic acids.

The guide nucleic acids used in Example 2 for CasM265466 were subject to modifications that contain 2′ O-Methyl modifications of the first 3 nucleotides and last 3 nucleotides, as well as phosphorothioate linkages between the first 4 nucleotides, and phosphorothioate linkages between the last 3 nucleotides. TABLE 19 provides the exemplary modified guide nucleic acids tested with CasM265466.

TABLE 19
Exemplary modified guide nucleic
acids for CasM265466

| | Target | Guide sequence | SEQ |
|---|---|---|---|
| Name | allele | (shown as RNA), (5′ to 3′) | ID NO |
| R13833- | NRAS | mA*mC*mA*GCUUAUUUGGAAGCUGAAAUG | 114 |
| M | WT | UGAGGUUUAUAACACUCACAAGAAUCCUGA | |
| | | AAAAGGAUGCCAAACCUCUUCUUGUCCAGC | |
| | | UGmU*mA*mU | |
| R13834- | NRAS | mA*mC*mA*GCUUAUUUGGAAGCUGAAAUG | 115 |
| M | Q61L | UGAGGUUUAUAACACUCACAAGAAUCCUGA | |
| | | AAAAGGAUGCCAAACCUCUUCUAGUCCAGC | |
| | | UGmU*mA*mU | |
| R13835- | NRAS | mA*mC*mA*GCUUAUUUGGAAGCUGAAAUG | 116 |
| M | Q61L | UGAGGUUUAUAACACUCACAAGAAUCCUGA | |
| | | AAAAGGAUGCCAAAGUCCAGCUGUAUCCAG | |
| | | UmA*mU*mG | |

Notes:M indicates that a guide contains modifications; m is
2′ O-Me modified sugar moiety;

HepG2 or control Hep3B cells were transfected with nuclease mRNA and guide nucleic acid at a 5 ug:500 pmol ratio. The transfected cells were then grown for 48 hours and indel activity of the effector protein/guide nucleic acid was analyzed by NGS.

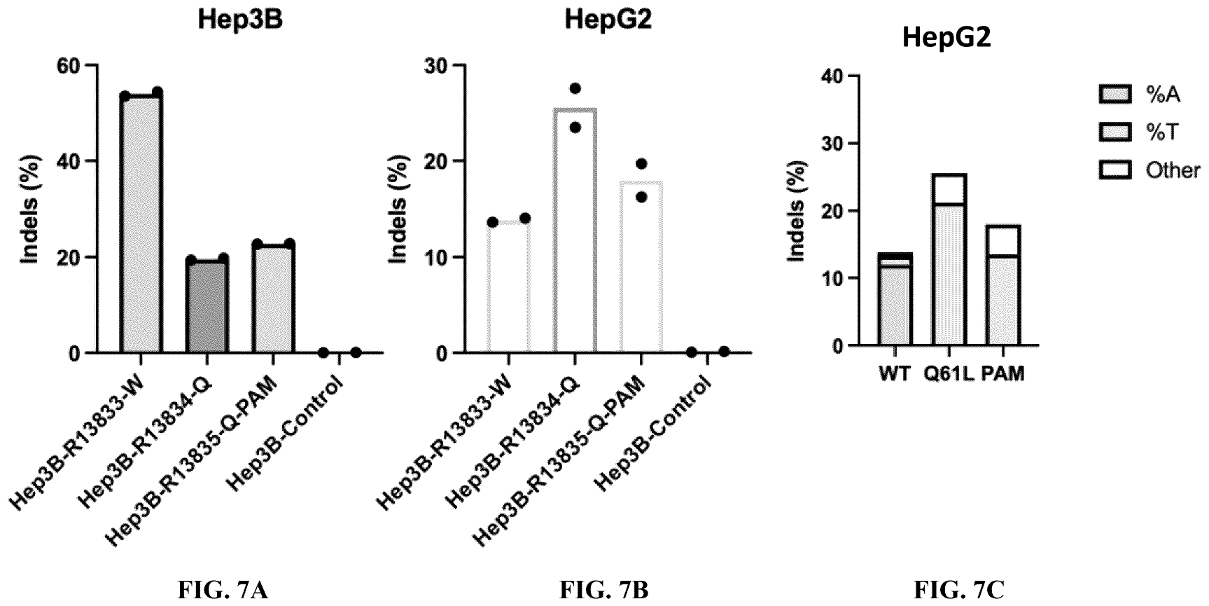

As shown in FIGS. 7A-7C, CasM265466 exhibited significant editing in both HepG2 and Hep3B cells. Further, CasM265466 displayed allele-specific editing capacity within a single cell, i.e., WT guides edited WT alleles and vice versa.

### Example 5: SNP-Specific KRAS Knockdown Using CasM.265466

Experiments were carried out to assess the specificity of CasM.265466 in knockdown of the KRAS gene in a human pancreas adenocarcinoma cell line by measuring indel activity.

The AsPC1 human PDAC cell line is a homozygous cell line that harbors 2 copies of KRAS G12D mutant alleles. BxPC3 is a control cell line with homozygous WT KRAS alleles. These two cell lines were used as models for this study.

PDAC or control BxPC3cells were transfected with nuclease mRNA and guide nucleic acid at a 5 μg:500 pmol ratio. The transfected cells were then grown for 48 hours and indel activity of the effector protein/guide nucleic acid was analyzed by NGS.

The nuclease mRNA encodes CasM.265466, CasPhi.12, or Cas9.

For CasM.265466, one pair of guides (the WT guide targeting KRAS WT and the G12D guide targeting KRAS G12D loci) was screened using mRNA nucleofection. The guide sequences are provided in TABLE 20.

TABLE 20
Exemplary guide nucleic acids for CasM265466

| Name | Target | Spacer Sequence | PAM |
|---|---|---|---|
| R5681 | KRAS WT | GUAGUUGGAGCUGGU | TGTG |
| | | GGCGU | |
| | | (SEQ ID NO: 79) | |
| R5682 | KRAS G12D | GUAGUUGGAGCUGAU | TGTG |
| | (mutation in | GGCGU | |
| | PAM + 14) | (SEQ ID NO: 80) | |

Handle sequence for CasM265466 (69 nt):
ACAGCUUAUUUGGAAGCUGAAAUGUGAGGUUUAUAACAC
UCACAAGAAUCCUGAAAAAGGAUGCCAAAC

The guide nucleic acids for CasM265466 were subject to modifications that contain 2′ O-Methyl modifications of the first 3 nucleotides and last 3 nucleotides, as well as phosphorothioate linkages between the first 4 nucleotides, and phosphorothioate linkages between the last 3 nucleotides. TABLE 21 provides the exemplary modified guide nucleic acids tested with CasM265466.

TABLE 21
Exemplary modified guide nucleic
acids for CasM265466

| | | Guide sequence | SEQ |
|---|---|---|---|
| Name | Target | (shown as RNA), (5′to 3′) | ID NO |
| R5681- | KRAS | mA*mC*mA*GCUUAUUUGGAAGCUGAAAUGU | 117 |
| M | WT | GAGGUUUAUAACACUCACAAGAAUCCUGAAA | |
| | | AAGGAUGCCAAACGUAGUUGGAGCUGGUGGm | |
| | | C*mG*mU | |
| R5682- | KRAS | mA*mC*mA*GCUUAUUUGGAAGCUGAAAUGU | 118 |
| M | G12D | GAGGUUUAUAACACUCACAAGAAUCCUGAAA | |
| | | AAGGAUGCCAAACGUAGUUGGAGCUGAUGGm | |
| | | C*mG*mU | |

Notes:
M indicates that a guide contains modifications; m is 2′ O-Me
modified sugar moiety; and the * denotes a PS linkage

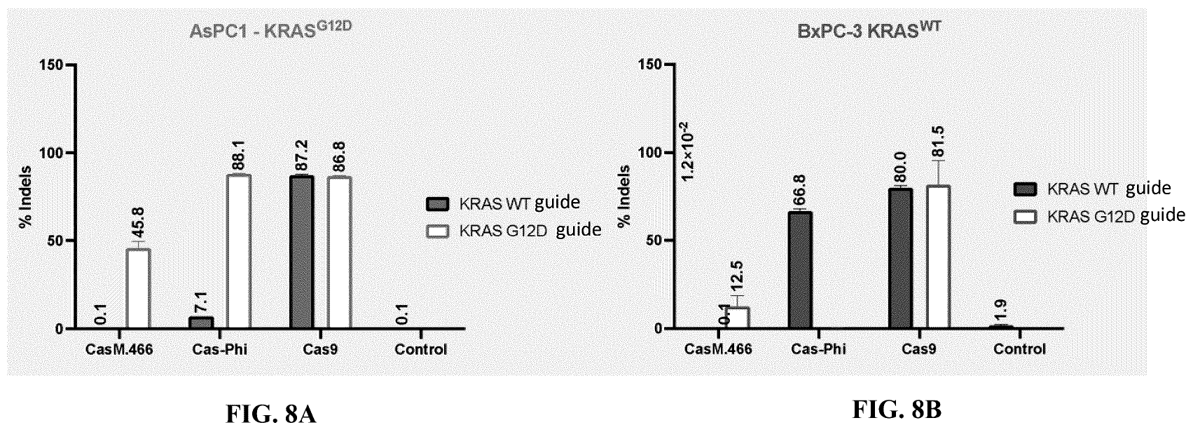

As shown in FIG. 8A and FIG. 8B, the WT guide for CasM.265466 showed no activity in both cell lines. In contrast, the KRAS G12D guide, when paired with CasM.265466, displayed a high degree of specificity for the KRAS G12D mutant (45.8% Indel formation in AsPC1-KRAS^{G12D} cells vs 12.5% Indel formation in BxPC-3 KRAS^{WT} cells), indicating that when combined with the KRAS G12D guide, CasM.265466 is capable of inducing a SNP-specific KRAS editing.

1-106. (canceled)

107. A method for allele-specific editing of a target gene in a cell, comprising contacting the cell with:
a) an effector protein comprising an amino acid sequence that is at least 90% identical to SEQ ID NO: 3, or a polynucleotide encoding the same, and
b) a guide nucleic acid or a polynucleotide encoding the same, wherein the guide nucleic acid comprises a sequence that is complementary to a target sequence in a mutant allele of the target gene.

108. The method of claim 107, wherein the effector protein comprises at least one amino acid substitution described in TABLE 5.

109. The method of claim 107, wherein the target sequence comprises a mutation relative to the wildtype allele.

110. The method of claim 109, wherein the mutation comprises a single nucleotide polymorphism (SNP).

111. The method of claim 109, wherein the mutation comprises a missense mutation.

112. The method of claim 107, wherein the effector protein cleaves the mutant allele, and wherein the effector protein does not cleave the wildtype allele.

113. The method of claim 107, wherein the effector protein cleaves the mutant allele more than it cleaves the wildtype allele.

114. The method of claim 107, comprising a partner protein or a nucleic acid encoding the same.

115. The method of claim 114, wherein the partner protein is fused or linked to the effector protein.

116. The method of claim 115, wherein the partner protein exerts allele specific gene regulation.

117. The method of claim 107, wherein the guide nucleic acid comprises a protein binding sequence comprising a sequence that is at least 80% identical to a sequence selected from SEQ ID NO: 89, SEQ ID NO: 90, and SEQ ID NO: 91.

118. The method of claim 107, wherein the guide nucleic acid comprises a protein binding sequence comprising a sequence that is at least 90% identical to a sequence selected from SEQ ID NO: 89, SEQ ID NO: 90, and SEQ ID NO: 91.

119. The method of claim 107, wherein the amino acid sequence is at least 95% identical to SEQ ID NO: 3.

120. The method of claim 107, comprising contacting the cell with an adenoviral associated viral (AAV) vector that comprises the polynucleotide encoding the effector protein and the polynucleotide encoding the guide nucleic acid.

121. The method of claim 107, wherein the target sequence is a eukaryotic sequence.

122. A cell, or population of cells, modified by the method of claim 107.

123. A method of modifying a NRAS gene, comprising contacting a cell with:
a) an effector protein comprising an amino acid sequence that is at least 90% identical to SEQ ID NO: 3, or a polynucleotide encoding the same, and
b) a guide nucleic acid or a polynucleotide encoding the same, wherein the guide nucleic acid comprises a sequence that is complementary to a target sequence of NRAS.

124. A method of modifying a KRAS gene, comprising contacting a cell with:
a) an effector protein comprising an amino acid sequence that is at least 90% identical to SEQ ID NO: 3, or a polynucleotide encoding the same, and
b) a guide nucleic acid or a polynucleotide encoding the same, wherein the guide nucleic acid comprises a sequence that is complementary to a target sequence of KRAS.


SEQUENCE LISTING (118 sequences)

SEQ ID NO: 1 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 2 - protein, 748 aa, synthetic construct
PKKKRKVGIHGVPAAMIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKK

SEQ ID NO: 3 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 4 - protein, 504 aa, synthetic construct
MDYKDHDGDYKDHDIDYKDDDDKMAPKKKRKVGIHGVPAAMSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKKRPAATKKAGQAKKKK

SEQ ID NO: 5 - protein, 15 aa, synthetic construct
PKKKRKVGIHGVPAA

SEQ ID NO: 6 - protein, 16 aa, synthetic construct
KRPAATKKAGQAKKKK

SEQ ID NO: 7 - protein, 4 aa, synthetic construct
  VARIANT 3: note = replace with R
KKKR

SEQ ID NO: 8 - protein, 7 aa, synthetic construct
  VARIANT 1: note = replace with R
  VARIANT 2..3: note = X is any amino acid
  VARIANT 6: note = X is any amino acid except D or E
  VARIANT 7: note = replace with R
PXXKRXK

SEQ ID NO: 9 - protein, 8 aa, synthetic construct
  VARIANT 4: note = W, F or Y
  VARIANT 3..6: note = X is any amino acid
KRXXXXAF

SEQ ID NO: 10 - protein, 7 aa, synthetic construct
  VARIANT 4: note = replace with P
  VARIANT 2..3: note = X is any amino acid
  VARIANT 4: note = replace with R
  VARIANT 7: note = X is any amino acid except D or E
RXXKRKX

SEQ ID NO: 11 - protein, 6 aa, synthetic construct
  VARIANT 5: note = K or R
  VARIANT 6: note = W, F or Y
LGKRXX

SEQ ID NO: 12 - protein, 6 aa, synthetic construct
  VARIANT 3: note = X is any amino acid
  VARIANT 4: note = replace with R
  VARIANT 5: note = replace with R
KRXKKK

SEQ ID NO: 13 - protein, 4 aa, synthetic construct
  VARIANT 2: note = replace with R
KKRK

SEQ ID NO: 14 - protein, 6 aa, synthetic construct
  VARIANT 3: note = X is any amino acid
  VARIANT 5: note = replace with R
  VARIANT 6: note = replace with R
KRXKKK

SEQ ID NO: 15 - protein, 6 aa, synthetic construct
  VARIANT 3: note = X is any amino acid
  VARIANT 5: note = replace with R
  VARIANT 6: note = replace with R
KRXKKK

SEQ ID NO: 16 - protein, 7 aa, synthetic construct
  VARIANT 3: note = X is any amino acid
  VARIANT 5: note = replace with R
  VARIANT 6: note = X is any amino acid
  VARIANT 7: note = replace with R
KRXKKXK

SEQ ID NO: 17 - protein, 7 aa, synthetic construct
  VARIANT 3: note = X is any amino acid
  VARIANT 5: note = replace with R
  VARIANT 6: note = X is any amino acid
  VARIANT 7: note = replace with R
KRXKKXK

SEQ ID NO: 18 - protein, 7 aa, synthetic construct
  VARIANT 3: note = X is any amino acid
  VARIANT 5: note = replace with R
  VARIANT 6: note = X is any amino acid
  VARIANT 7: note = replace with R
KRXKKXK

SEQ ID NO: 19 - protein, 16 aa, synthetic construct
APKKKRKVGIHGVPAA

SEQ ID NO: 20 - protein, 20 aa, synthetic construct
  VARIANT 4: note = X is any amino acid
  VARIANT 8: note = X is any amino acid
  VARIANT 11: note = X is any amino acid
  VARIANT 15: note = X is any amino acid
  VARIANT 19: note = X is any amino acid
GLFXALLXLLXSLWXLLLXA

SEQ ID NO: 21 - protein, 20 aa, synthetic construct
GLFHALLHLLHSLWHLLLHA

SEQ ID NO: 22 - protein, 36 aa, synthetic construct
SDLYIERGGDPRDVHQQVETKPKGKRKSEIRILKIR

SEQ ID NO: 23 - protein, 31 aa, synthetic construct
SDYIVDHGGDPEKVFFETKSKKDKTKRYKRR

SEQ ID NO: 24 - protein, 717 aa, synthetic construct
MIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 25 - protein, 775 aa, synthetic construct
MDYKDHDGDYKDHDIDYKDDDDKMAPKKKRKVGIHGVPAAMIKPTVSQFLTPGFKLIRNHSRTAGRKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKGRRPRKRPARQKRKRNS

SEQ ID NO: 26 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIANLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 27 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 28 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSRHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 29 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYRNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 30 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQRPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 31 - protein, 717 aa, synthetic construct
MIKPTVSQFLTPGFKLIRNHSRTAGLKLKNEGEEACKRFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 32 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLRQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 33 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLRKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 34 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLREHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 35 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVRPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 36 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDRVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 37 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 38 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVNVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 39 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNAAIDVATENLATVAITAQSMPKPTCERSGDAKKPVRARKAKAPEFHDKLAPSYTVVLREAV

SEQ ID NO: 40 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNANIDVATENLATVAITAQSMPKPTCERSGDAKKPVRARKAKAPEFHDKLAPSYTVVLREAV

SEQ ID NO: 41 - protein, 698 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 42 - protein, 704 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEGSSGDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 43 - protein, 717 aa, synthetic construct
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 44 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 45 - protein, 448 aa, synthetic construct
MSVLTRKVQLIPVGDKEERDRVYKYLRDGIEAQNRAMNLYMSGLYFAAINEASKEDRWELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 46 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMKMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 47 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKWKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 48 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPLDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 49 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQKIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 50 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHMVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 51 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFFGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 52 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDKEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 53 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKKIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 54 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSKGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 55 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEKNWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 56 - protein, 448 aa, synthetic construct; identical to SEQ ID NO: 55

SEQ ID NO: 57 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLQNLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 58 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVALGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 59 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNAAFNAARNIAMSTEFQSGKKTKKQKKEQHENK

SEQ ID NO: 60 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLENLEGIRDDVKNEWLLSNWSYYQLQQYITYKAKTYGIEVRKINPYHTSQRCSCCGYEDAGNRPKKEKGQAYFKCLKCGEEMNANFNAARNIAMSTEFQSGKKTKKQKKEQHENK

SEQ ID NO: 61 - protein, 448 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMDIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLANLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENK

SEQ ID NO: 62 - protein, 17 aa, synthetic construct
MAPKKKRKVGIHGVPAA

SEQ ID NO: 63 - protein, 16 aa, synthetic construct
KRPAATKKAGQAKKKK

SEQ ID NO: 64 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 65 - protein, 664 aa, synthetic construct
MSVLTRKVQLIPVGDKEERDRVYKYLRDGIEAQNRAMNLYMSGLYFAAINEASKEDRKELNQLYSRIATSSKGSAYTTDIEFPTGLASTSTLSMAVRQDFTKSLKDGLMYGRVSLPTYRKDNPLFVDVRFVALRGTKQKYNGLYHEYKSHTEFLDNLYSSDLKVYIKFANDITFQVIFGNPRKSSALRSEFQNIFEEYYKVCQSSIQFSGTKIILNMAMRIPDKEIELDEDVCVGVDLGIAIPAVCALNKNRYSRVSIGSKEDFLRVRTKIRNQRKRLQTNLKSSNGGHGRKKKMKPMDRFRDYEANWVQNYNHYVSRQVVDFAVKNKAKYINLQNLEGIRDDVKNEWLLSNWSYYQLQQYITYKAKTYGIEVRKINPYHTSQRCSCCGYEDAGNRPKKEKGQAYFKCLKCGEEMNADFNAARNIAMSTEFQSGKKTKKQKKEQHENKGSSGGSPAGSPTSTEEGTSESATPESGPGTSTEPSEGSAPGSPAGSGGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 66 - protein, 933 aa, synthetic construct
MIKPTVSQFLTPGFKLIRNHSRTAGKKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVGSSGGSPAGSPTSTEEGTSESATPESGPGTSTEPSEGSAPGSPAGSGGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 67 - protein, 50 aa, synthetic construct
GSSGGSPAGSPTSTEEGTSESATPESGPGTSTEPSEGSAPGSPAGSGGGS

SEQ ID NO: 68 - RNA, 20 nt, synthetic construct
  source 1..20: mol_type = other RNA
ttgtccagctgtatccagta

SEQ ID NO: 69 - RNA, 19 nt, synthetic construct
  source 1..19: mol_type = other RNA
ttgtccagctgtatccagt

SEQ ID NO: 70 - RNA, 18 nt, synthetic construct
  source 1..18: mol_type = other RNA
ttgtccagctgtatccag

SEQ ID NO: 71 - RNA, 17 nt, synthetic construct
  source 1..17: mol_type = other RNA
ttgtccagctgtatcca

SEQ ID NO: 72 - RNA, 20 nt, synthetic construct
  source 1..20: mol_type = other RNA
tagtccagctgtatccagta

SEQ ID NO: 73 - RNA, 19 nt, synthetic construct
  source 1..19: mol_type = other RNA
tagtccagctgtatccagt

SEQ ID NO: 74 - RNA, 18 nt, synthetic construct
  source 1..18: mol_type = other RNA
tagtccagctgtatccag

SEQ ID NO: 75 - RNA, 17 nt, synthetic construct
  source 1..17: mol_type = other RNA
tagtccagctgtatcca

SEQ ID NO: 76 - RNA, 20 nt, synthetic construct
  source 1..20: mol_type = other RNA
ctcttcttgtccagctgtat

SEQ ID NO: 77 - RNA, 20 nt, synthetic construct
  source 1..20: mol_type = other RNA
ctcttctagtccagctgtat

SEQ ID NO: 78 - RNA, 20 nt, synthetic construct
  source 1..20: mol_type = other RNA
gtccagctgtatccagtatg

SEQ ID NO: 79 - RNA, 20 nt, synthetic construct
  source 1..20: mol_type = other RNA
gtagttggagctggtggcgt

SEQ ID NO: 80 - RNA, 20 nt, synthetic construct
  source 1..20: mol_type = other RNA
gtagttggagctgatggcgt

SEQ ID NO: 81 - RNA, 36 nt, synthetic construct
  source 1..36: mol_type = other RNA
ctttcaagactaatagattgctccttacgaggagac

SEQ ID NO: 82 - RNA, 24 nt, synthetic construct
  source 1..24: mol_type = other RNA
atagattgctccttacgaggagac

SEQ ID NO: 83 - RNA, 23 nt, synthetic construct
  source 1..23: mol_type = other RNA
tagattgctccttacgaggagac

SEQ ID NO: 84 - RNA, 22 nt, synthetic construct
  source 1..22: mol_type = other RNA
agattgctccttacgaggagac

SEQ ID NO: 85 - RNA, 21 nt, synthetic construct
  source 1..21: mol_type = other RNA
gattgctccttacgaggagac

SEQ ID NO: 86 - RNA, 20 nt, synthetic construct
  source 1..20: mol_type = other RNA
attgctccttacgaggagac

SEQ ID NO: 87 - RNA, 24 nt, synthetic construct
  source 1..24: mol_type = other RNA
gtagattgctccttacgaggagac

SEQ ID NO: 88 - RNA, 30 nt, synthetic construct
  source 1..30: mol_type = other RNA
agactaatagattgctccttacgaggagac

SEQ ID NO: 89 - RNA, 13 nt, synthetic construct
  source 1..13: mol_type = other RNA
aaggatgccaaac

SEQ ID NO: 90 - RNA, 69 nt, synthetic construct
  source 1..69: mol_type = other RNA
acagcttatttggaagctgaaatgtgaggtttataacactcacaagaatcctgaaaaaggatgccaaac

SEQ ID NO: 91 - RNA, 52 nt, synthetic construct
  source 1..52: mol_type = other RNA
acagcttatttggaagctgaaatgtgaggtttataacactcacaagaatcct

SEQ ID NO: 92 - skipped

SEQ ID NO: 93 - DNA, 28 bp, Homo sapiens
  source 1..28: mol_type = genomic DNA
ggacatactggatacagctggacaagaa

SEQ ID NO: 94 - DNA, 28 bp, Homo sapiens
  source 1..28: mol_type = genomic DNA
ggacatactggatacagctggactagaa

SEQ ID NO: 95 - RNA, 56 nt, synthetic construct
  source 1..56: mol_type = other RNA
ctttcaagactaatagattgctccttacgaggagacttgtccagctgtatccagta

SEQ ID NO: 96 - RNA, 55 nt, synthetic construct
  source 1..55: mol_type = other RNA
ctttcaagactaatagattgctccttacgaggagacttgtccagctgtatccagt

SEQ ID NO: 97 - RNA, 38 nt, synthetic construct
  source 1..38: mol_type = other RNA
attgctccttacgaggagacttgtccagctgtatccag

SEQ ID NO: 98 - RNA, 37 nt, synthetic construct
  source 1..37: mol_type = other RNA
attgctccttacgaggagacttgtccagctgtatcca

SEQ ID NO: 99 - RNA, 56 nt, synthetic construct
  source 1..56: mol_type = other RNA
ctttcaagactaatagattgctccttacgaggagactagtccagctgtatccagta

SEQ ID NO: 100 - RNA, 55 nt, synthetic construct
  source 1..55: mol_type = other RNA
ctttcaagactaatagattgctccttacgaggagactagtccagctgtatccagt

SEQ ID NO: 101 - RNA, 38 nt, synthetic construct
  source 1..38: mol_type = other RNA
attgctccttacgaggagactagtccagctgtatccag

SEQ ID NO: 102 - RNA, 37 nt, synthetic construct
  source 1..37: mol_type = other RNA
attgctccttacgaggagactagtccagctgtatcca

SEQ ID NO: 103 - RNA, 89 nt, synthetic construct
  source 1..89: mol_type = other RNA
acagcttatttggaagctgaaatgtgaggtttataacactcacaagaatcctgaaaaaggatgccaaacctcttcttgtccagctgtat

SEQ ID NO: 104 - RNA, 89 nt, synthetic construct
  source 1..89: mol_type = other RNA
acagcttatttggaagctgaaatgtgaggtttataacactcacaagaatcctgaaaaaggatgccaaacctcttctagtccagctgtat

SEQ ID NO: 105 - RNA, 88 nt, synthetic construct
  source 1..88: mol_type = other RNA
acagcttatttggaagctgaaatgtgaggtttataacactcacaagaatcctgaaaaaggatgccaaagtccagctgtatccagtatg

SEQ ID NO: 106 - RNA, 89 nt, synthetic construct
  source 1..89: mol_type = other RNA
acagcttatttggaagctgaaatgtgaggtttataacactcacaagaatcctgaaaaaggatgccaaacgtagttggagctggtggcgt

SEQ ID NO: 107 - RNA, 89 nt, synthetic construct
  source 1..89: mol_type = other RNA
acagcttatttggaagctgaaatgtgaggtttataacactcacaagaatcctgaaaaaggatgccaaacgtagttggagctgatggcgt

SEQ ID NO: 108 - RNA, 56 nt, synthetic construct; identical to SEQ ID NO: 95
  modified_base 1..4: mod_base = OTHER; note = linked via phosphorothioate bond
  modified_base 1: mod_base = cm
  modified_base 2..3: mod_base = um
  modified_base 54..56: mod_base = OTHER; note = linked via phosphorothioate bond
  modified_base 54: mod_base = gm
  modified_base 55: mod_base = um
  modified_base 56: mod_base = OTHER; note = 2'-O-methyladenosine
  source 1..56: mol_type = other RNA

SEQ ID NO: 109 - RNA, 55 nt, synthetic construct; identical to SEQ ID NO: 96
  modified_base 1..4: mod_base = OTHER; note = linked via phosphorothioate bond
  modified_base 1: mod_base = cm
  modified_base 2..3: mod_base = um
  modified_base 53..55: mod_base = OTHER; note = linked via phosphorothioate bond
  modified_base 53: mod_base = OTHER; note = 2'-O-methyladenosine
  modified_base 54: mod_base = gm
  modified_base 55: mod_base = um
  source 1..55: mol_type = other RNA

SEQ ID NO: 110 - RNA, 54 nt, synthetic construct
  modified_base 1..4: mod_base = OTHER; note = linked via phosphorothioate bond
  modified_base 1: mod_base = cm
  modified_base 2..3: mod_base = um
  modified_base 52..54: mod_base = OTHER; note = linked via phosphorothioate bond
  modified_base 52: mod_base = cm
  modified_base 53: mod_base = OTHER; note = 2'-O-methyladenosine
  modified_base 54: mod_base = gm
  source 1..54: mol_type = other RNA
ctttcaagactaatagattgctccttacgaggagacttgtccagctgtatccag

SEQ ID NO: 111 - RNA, 56 nt, synthetic construct; identical to SEQ ID NO: 99
  modified_base 1..4: mod_base = OTHER; note = linked via phosphorothioate bond
  modified_base 1: mod_base = cm
  modified_base 2..3: mod_base = um
  modified_base 54..56: mod_base = OTHER; note = linked via phosphorothioate bond
  modified_base 54: mod_base = gm
  modified_base 55: mod_base = um
  modified_base 56: mod_base = OTHER; note = 2'-O-methyladenosine
  source 1..56: mol_type = other RNA

SEQ ID NO: 112 - RNA, 55 nt, synthetic construct; identical to SEQ ID NO: 100
  modified_base 1..4: mod_base = OTHER; note = linked via phosphorothioate bond
  modified_base 1: mod_base = cm
  modified_base 2..3: mod_base = um
  modified_base 53..55: mod_base = OTHER; note = linked via phosphorothioate bond
  modified_base 53: mod_base = OTHER; note = 2'-O-methyladenosine
  modified_base 54: mod_base = gm
  modified_base 55: mod_base = um
  source 1..55: mol_type = other RNA

SEQ ID NO: 113 - RNA, 54 nt, synthetic construct
  modified_base 1..4: mod_base = OTHER; note = linked via phosphorothioate bond
  modified_base 1: mod_base = cm
  modified_base 2..3: mod_base = um
  modified_base 52..54: mod_base = OTHER; note = linked via phosphorothioate bond
  modified_base 52: mod_base = cm
  modified_base 53: mod_base = OTHER; note = 2'-O-methyladenosine
  modified_base 54: mod_base = gm
  source 1..54: mol_type = other RNA
ctttcaagactaatagattgctccttacgaggagactagtccagctgtatccag

SEQ ID NO: 114 - RNA, 89 nt, synthetic construct; identical to SEQ ID NO: 103
  modified_base 1..4: mod_base = OTHER; note = linked via phosphorothioate bond
  modified_base 1: mod_base = OTHER; note = 2'-O-methyladenosine
  modified_base 2: mod_base = cm
  modified_base 3: mod_base = OTHER; note = 2'-O-methyladenosine
  modified_base 87..89: mod_base = OTHER; note = linked via phosphorothioate bond
  modified_base 87: mod_base = um
  modified_base 88: mod_base = OTHER; note = 2'-O-methyladenosine
  modified_base 89: mod_base = um
  source 1..89: mol_type = other RNA

SEQ ID NO: 115 - RNA, 89 nt, synthetic construct; identical to SEQ ID NO: 104
  modified_base 1..4: mod_base = OTHER; note = linked via phosphorothioate bond
  modified_base 1: mod_base = OTHER; note = 2'-O-methyladenosine
  modified_base 2: mod_base = cm
  modified_base 3: mod_base = OTHER; note = 2'-O-methyladenosine
  modified_base 87..89: mod_base = OTHER; note = linked via phosphorothioate bond
  modified_base 87: mod_base = um
  modified_base 88: mod_base = OTHER; note = 2'-O-methyladenosine
  modified_base 89: mod_base = um
  source 1..89: mol_type = other RNA

SEQ ID NO: 116 - RNA, 88 nt, synthetic construct; identical to SEQ ID NO: 105
  modified_base 1..4: mod_base = OTHER; note = linked via phosphorothioate bond
  modified_base 1: mod_base = OTHER; note = 2'-O-methyladenosine
  modified_base 2: mod_base = cm
  modified_base 3: mod_base = OTHER; note = 2'-O-methyladenosine
  modified_base 86..88: mod_base = OTHER; note = linked via phosphorothioate bond
  modified_base 86: mod_base = OTHER; note = 2'-O-methyladenosine
  modified_base 87: mod_base = um
  modified_base 88: mod_base = gm
  source 1..88: mol_type = other RNA

SEQ ID NO: 117 - RNA, 89 nt, synthetic construct; identical to SEQ ID NO: 106
  modified_base 1..4: mod_base = OTHER; note = linked via phosphorothioate bond
  modified_base 1: mod_base = OTHER; note = 2'-O-methyladenosine
  modified_base 2: mod_base = cm
  modified_base 3: mod_base = OTHER; note = 2'-O-methyladenosine
  modified_base 87..89: mod_base = OTHER; note = linked via phosphorothioate bond
  modified_base 87: mod_base = cm
  modified_base 88: mod_base = gm
  modified_base 89: mod_base = um
  source 1..89: mol_type = other RNA

SEQ ID NO: 118 - RNA, 89 nt, synthetic construct; identical to SEQ ID NO: 107
  modified_base 1..4: mod_base = OTHER; note = linked via phosphorothioate bond
  modified_base 1: mod_base = OTHER; note = 2'-O-methyladenosine
  modified_base 2: mod_base = cm
  modified_base 3: mod_base = OTHER; note = 2'-O-methyladenosine
  modified_base 87..89: mod_base = OTHER; note = linked via phosphorothioate bond
  modified_base 87: mod_base = cm
  modified_base 88: mod_base = gm
  modified_base 89: mod_base = um
  source 1..89: mol_type = other RNA